SEQUENCE LISTING (158 sequences)

SEQ ID NO: 1 - protein, 1368 aa, Artificial Sequence [sequence omitted]

SEQ ID NO: 2 - RNA, 100 nt, Artificial Sequence
  Synthetic
  misc_feature (1)..(4): modified with 2′-O-methyl phosphorothioate
  misc_feature (97)..(100): modified with 2′-O-methyl phosphorothioate
agagcaacagugcuguggccguuuuagagcuagaaauagcaaguuaaaauaaggcuaguccguuaucaacuugaaaaaguggcaccgagucggugcuuuu

SEQ ID NO: 3 - RNA, 100 nt, Artificial Sequence; identical to SEQ ID NO: 2
  Synthetic

SEQ ID NO: 4 - RNA, 20 nt, Artificial Sequence
  Synthetic
  misc_feature (1)..(4): modified with 2′-O-methyl phosphorothioate
agagcaacagugcuguggcc

SEQ ID NO: 5 - RNA, 20 nt, Artificial Sequence
  Synthetic
agagcaacagugcuguggcc

SEQ ID NO: 6 - RNA, 100 nt, Artificial Sequence
  Synthetic
  misc_feature (1)..(4): modified with 2′-O-methyl phosphorothioate
  misc_feature (97)..(100): modified with 2′-O-methyl phosphorothioate
gcuacucucucuuucuggccguuuuagagcuagaaauagcaaguuaaaauaaggcuaguccguuaucaacuugaaaaaguggcaccgagucggugcuuuu

SEQ ID NO: 7 - RNA, 100 nt, Artificial Sequence; identical to SEQ ID NO: 6
  Synthetic

SEQ ID NO: 8 - RNA, 20 nt, Artificial Sequence
  Synthetic
  misc_feature (1)..(4): modified with 2′-O-methyl phosphorothioate
gcuacucucucuuucuggcc

SEQ ID NO: 9 - RNA, 20 nt, Artificial Sequence
  Synthetic
gcuacucucucuuucuggcc

SEQ ID NO: 10 - RNA, 100 nt, Artificial Sequence
  Synthetic
  misc_feature (1)..(4): modified with 2′-O-methyl phosphorothioate
  misc_feature (97)..(100): modified with 2′-O-methyl phosphorothioate
gcuuuggucccauuggucgcguuuuagagcuagaaauagcaaguuaaaauaaggcuaguccguuaucaacuugaaaaaguggcaccgagucggugcuuuu

SEQ ID NO: 11 - RNA, 100 nt, Artificial Sequence; identical to SEQ ID NO: 10
  Synthetic

SEQ ID NO: 12 - RNA, 20 nt, Artificial Sequence
  Synthetic
  misc_feature (1)..(4): modified with 2′-O-methyl phosphorothioate
gcuuuggucccauuggucgc

SEQ ID NO: 13 - RNA, 20 nt, Artificial Sequence
  Synthetic
gcuuuggucccauuggucgc

SEQ ID NO: 14 - RNA, 100 nt, Artificial Sequence
  Synthetic
  misc_feature (1)..(4): modified with 2′-O-methyl phosphorothioate
  misc_feature (1)..(3): modified with 2′-O-methylation
  misc_feature (97)..(99): modified with 2′-O-methylation
  misc_feature (97)..(100): modified with 2′-O-methyl phosphorothioate
acgacgcguggguggcaagcguuuuagagcuagaaauagcaaguuaaaauaaggcuaguccguuaucaacuugaaaaaguggcaccgagucggugcuuuu

SEQ ID NO: 15 - RNA, 101 nt, Artificial Sequence
  Synthetic
acgacgcguggguggcaagcguuuuagagcuagaaauagcaaguuaaaauaaggcuaguccguuaucaacuugaaaaaguggcaccgagucggugcmuuuu

SEQ ID NO: 16 - RNA, 20 nt, Artificial Sequence
  Synthetic
  misc_feature (1)..(4): modified with 2′-O-methyl phosphorothioate
  misc_feature (1)..(3): modified with 2′-O-methylation
acgacgcguggguggcaagc

SEQ ID NO: 17 - DNA, 20 bp, Artificial Sequence
  Synthetic
acgacgcgtgggtggcaagc

SEQ ID NO: 18 - RNA, 106 nt, Artificial Sequence
  Synthetic
  misc_feature (1)..(4): modified with 2′-O-methyl phosphorothioate
  misc_feature (1)..(3): modified with 2′-O-methylation
  misc_feature (103)..(105): modified with 2′-O-methylation
  misc_feature (103)..(106): modified with 2′-O-methyl phosphorothioate
ugcgcaggauuucugguugucacaggguuuuagagcuagaaauagcaaguuaaaauaaggcuaguccguuaucaacuugaaaaaguggcaccgagucggugcuuuu

SEQ ID NO: 19 - RNA, 107 nt, Artificial Sequence
  Synthetic
ugcgcaggauuucugguugucacaggguuuuagagcuagaaauagcaaguuaaaauaaggcuaguccguuaucaacuugaaaaaguggcaccgagucggugcmuuuu

SEQ ID NO: 20 - RNA, 29 nt, Artificial Sequence
  Synthetic
  misc_feature (1)..(4): modified with 2′-O-methyl phosphorothioate
  misc_feature (1)..(3): modified with 2′-O-methylation
mumgmcgcaggauuucugguugucacagg

SEQ ID NO: 21 - RNA, 26 nt, Artificial Sequence
  Synthetic
ugcgcaggauuucugguugucacagg

SEQ ID NO: 22 - RNA, 100 nt, Artificial Sequence
  Synthetic
  misc_feature (1)..(4): modified with 2′-O-methyl phosphorothioate
  misc_feature (97)..(100): modified with 2′-O-methyl phosphorothioate
cauuaggaccugcuccuagaguuuuagagcuagaaauagcaaguuaaaauaaggcuaguccguuaucaacuugaaaaaguggcaccgagucggugcuuuu

SEQ ID NO: 23 - RNA, 100 nt, Artificial Sequence; identical to SEQ ID NO: 22
  Synthetic

SEQ ID NO: 24 - RNA, 20 nt, Artificial Sequence
  Synthetic
  misc_feature (1)..(4): modified with 2′-O-methyl phosphorothioate
cauuaggaccugcuccuaga

SEQ ID NO: 25 - RNA, 20 nt, Artificial Sequence
  Synthetic
cauuaggaccugcuccuaga

SEQ ID NO: 26 - DNA, 23 bp, Artificial Sequence
  Synthetic
gctttggtcccattggtcgcggg

SEQ ID NO: 27 - DNA, 20 bp, Artificial Sequence
  Synthetic
gctttggtcccattggtcgc

SEQ ID NO: 28 - DNA, 23 bp, Artificial Sequence
  Synthetic
agagcaacagtgctgtggcctgg

SEQ ID NO: 29 - DNA, 20 bp, Artificial Sequence
  Synthetic
agagcaacagtgctgtggcc

SEQ ID NO: 30 - DNA, 23 bp, Artificial Sequence
  Synthetic
gctactctctctttctggcctgg

SEQ ID NO: 31 - DNA, 20 bp, Artificial Sequence
  Synthetic
gctactctctctttctggcc

SEQ ID NO: 32 - DNA, 23 bp, Artificial Sequence
  Synthetic
acgacgcgtgggtggcaagcggg

SEQ ID NO: 33 - DNA, 20 bp, Artificial Sequence
  Synthetic
acgacgcgtgggtggcaagc

SEQ ID NO: 34 - DNA, 29 bp, Artificial Sequence
  Synthetic
tgcgcaggatttctggttgtcacaggagg

SEQ ID NO: 35 - DNA, 26 bp, Artificial Sequence
  Synthetic
tgcgcaggatttctggttgtcacagg

SEQ ID NO: 36 - DNA, 23 bp, Artificial Sequence
  Synthetic
cattaggacctgctcctagatgg

SEQ ID NO: 37-43 - skipped (7 entries)

SEQ ID NO: 44 - DNA, 20 bp, Artificial Sequence
  Synthetic
cattaggacctgctcctaga

SEQ ID NO: 45 - RNA, 100 nt, Artificial Sequence
  Synthetic
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugcuuuu

SEQ ID NO: 46 - RNA, 96 nt, Artificial Sequence
  Synthetic
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugc

SEQ ID NO: 47 - RNA, 114 nt, Artificial Sequence
  Synthetic
  misc_feature (1)..(17): n is a, c, g, or u
  misc_feature (18)..(30): may be absent
  misc_feature (108)..(114): may be absent
nnnnnnnnnnnnnnnnnnnnnnnnnnnnnnguuuuagagcuagaaauagcaaguuaaaauaaggcuaguccguuaucaacuugaaaaaguggcaccgagucggugcuuuuuuuu

SEQ ID NO: 48 - DNA, 19 bp, Artificial Sequence
  Synthetic
aagagcaacaaatctgact

SEQ ID NO: 49 - DNA, 39 bp, Artificial Sequence
  Synthetic
aagagcaacagtgctgtgcctggagcaacaaatctgact

SEQ ID NO: 50 - DNA, 33 bp, Artificial Sequence
  Synthetic
aagagcaacagtgctggagcaacaaatctgact

SEQ ID NO: 51 - DNA, 34 bp, Artificial Sequence
  Synthetic
aagagcaacagtgcctggagcaacaaatctgact

SEQ ID NO: 52 - DNA, 19 bp, Artificial Sequence
  Synthetic
aagagcaacagtgctgact

SEQ ID NO: 53 - DNA, 41 bp, Artificial Sequence
  Synthetic
aagagcaacagtgctgtgggcctggagcaacaaatctgact

SEQ ID NO: 54 - skipped

SEQ ID NO: 55 - DNA, 38 bp, Artificial Sequence
  Synthetic
aagagcaacagtgctggcctggagcaacaaatctgact

SEQ ID NO: 56 - DNA, 41 bp, Artificial Sequence
  Synthetic
aagagcaacagtgctgtgtgcctggagcaacaaatctgact

SEQ ID NO: 57 - DNA, 79 bp, Artificial Sequence
  Synthetic
cgtggccttagctgtgctcgcgctactctctctttctgcctggaggctatccagcgtgagtctctcctaccctcccgct

SEQ ID NO: 58 - DNA, 78 bp, Artificial Sequence
  Synthetic
cgtggccttagctgtgctcgcgctactctctctttcgcctggaggctatccagcgtgagtctctcctaccctcccgct

SEQ ID NO: 59 - DNA, 75 bp, Artificial Sequence
  Synthetic
cgtggccttagctgtgctcgcgctactctctctttctggaggctatccagcgtgagtctctcctaccctcccgct

SEQ ID NO: 60 - DNA, 84 bp, Artificial Sequence
  Synthetic
cgtggccttagctgtgctcgcgctactctctctttctggatagcctggaggctatccagcgtgagtctctcctaccctcccgct

SEQ ID NO: 61 - DNA, 55 bp, Artificial Sequence
  Synthetic
cgtggccttagctgtgctcgcgctatccagcgtgagtctctcctaccctcccgct

SEQ ID NO: 62 - DNA, 82 bp, Artificial Sequence
  Synthetic
cgtggccttagctgtgctcgcgctactctctctttctgtggcctggaggctatccagcgtgagtctctcctaccctcccgct

SEQ ID NO: 63 - DNA, 58 bp, Artificial Sequence
  Synthetic
cacaccacgaggcagatcaccaagcccgcgcaatgggaccaaagcagcccgcaggacg

SEQ ID NO: 64 - DNA, 61 bp, Artificial Sequence
  Synthetic
cacaccacgaggcagatcaccaagcccgcgaaccaatgggaccaaagcagcccgcaggacg

SEQ ID NO: 65 - DNA, 48 bp, Artificial Sequence
  Synthetic
cacaccacgaggcagatcaccaatgggaccaaagcagcccgcaggacg

SEQ ID NO: 66 - DNA, 59 bp, Artificial Sequence
  Synthetic
cacaccacgaggcagatcaccaagcccgcgccaatgggaccaaagcagcccgcaggacg

SEQ ID NO: 67 - DNA, 59 bp, Artificial Sequence
  Synthetic
cacaccacgaggcagatcaccaagcccgcaccaatgggaccaaagcagcccgcaggacg

SEQ ID NO: 68 - DNA, 35 bp, Artificial Sequence
  Synthetic
cacaccacgaggcagatcaccaagcccgcaggacg

SEQ ID NO: 69 - DNA, 21 bp, Artificial Sequence
  Synthetic
gtgggtggcaaagcgggtggt

SEQ ID NO: 70 - DNA, 19 bp, Artificial Sequence
  Synthetic
gtgggtggcagcgggtggt

SEQ ID NO: 71 - DNA, 21 bp, Artificial Sequence
  Synthetic
gtgggtggcatagcgggtggt

SEQ ID NO: 72 - DNA, 18 bp, Artificial Sequence
  Synthetic
gtgggtggagcgggtggt

SEQ ID NO: 73 - DNA, 21 bp, Artificial Sequence
  Synthetic
acctgctccttagatgggtat

SEQ ID NO: 74 - DNA, 19 bp, Artificial Sequence
  Synthetic
acctgctccagatgggtat

SEQ ID NO: 75 - DNA, 17 bp, Artificial Sequence
  Synthetic
acctgctagatgggtat

SEQ ID NO: 76 - DNA, 10 bp, Artificial Sequence
  Synthetic
acctgggtat

SEQ ID NO: 77 - DNA, 19 bp, Artificial Sequence
  Synthetic
acctgctctagatgggtat

SEQ ID NO: 78 - DNA, 16 bp, Artificial Sequence
  Synthetic
acctgtagatgggtat

SEQ ID NO: 79 - DNA, 18 bp, Artificial Sequence
  Synthetic
acctgctcctatgggtat

SEQ ID NO: 80 - protein, 22 aa, Artificial Sequence
  Synthetic
MLLLVTSLLLCELPHPAFLLIP

SEQ ID NO: 81 - protein, 21 aa, Artificial Sequence
  Synthetic
MALPVTALLLPLALLLHAARP

SEQ ID NO: 82 - protein, 23 aa, Artificial Sequence
  Synthetic
IYIWAPLAGTCGVLLLSLVITLY

SEQ ID NO: 83 - DNA, 126 bp, Artificial Sequence
  Synthetic
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 84 - protein, 42 aa, Artificial Sequence
  Synthetic
KRGRKKLLYIFKQPFMRPVQTTQEEDGCSCRFPEEEEGGCEL

SEQ ID NO: 85 - DNA, 120 bp, Artificial Sequence
  Synthetic
tcaaagcggagtaggttgttgcattccgattacatgaatatgactcctcgccggcctgggccgacaagaaaacattaccaaccctatgcccccccacgagacttcgctgcgtacaggtcc

SEQ ID NO: 86 - protein, 40 aa, Artificial Sequence
  Synthetic
SKRSRLLHSDYMNMTPRRPGPTRKHYQPYAPPRDFAAYRS

SEQ ID NO: 87 - DNA, 336 bp, Artificial Sequence
  Synthetic
cgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 88 - protein, 112 aa, Artificial Sequence
  Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 11 aa, Artificial Sequence
  Synthetic
RASQDISKYLN

SEQ ID NO: 90 - protein, 7 aa, Artificial Sequence
  Synthetic
HTSRLHS

SEQ ID NO: 91 - protein, 9 aa, Artificial Sequence
  Synthetic
QQGNTLPYT

SEQ ID NO: 92 - protein, 5 aa, Artificial Sequence
  Synthetic
DYGVS

SEQ ID NO: 93 - protein, 16 aa, Artificial Sequence
  Synthetic
VIWGSETTYYNSALKS

SEQ ID NO: 94 - protein, 12 aa, Artificial Sequence
  Synthetic
HYYYGGSYAMDY

SEQ ID NO: 95 - protein, 11 aa, Artificial Sequence
  Synthetic
RASQDISKYLN

SEQ ID NO: 96 - protein, 7 aa, Artificial Sequence
  Synthetic
HTSRLHS

SEQ ID NO: 97 - protein, 9 aa, Artificial Sequence
  Synthetic
QQGNTLPYT

SEQ ID NO: 98 - protein, 7 aa, Artificial Sequence
  Synthetic
GVSLPDY

SEQ ID NO: 99 - protein, 5 aa, Artificial Sequence
  Synthetic
WGSET

SEQ ID NO: 100 - protein, 12 aa, Artificial Sequence
  Synthetic
HYYYGGSYAMDY

SEQ ID NO: 101 - DNA, 1518 bp, Artificial Sequence
  Synthetic
atgcttcttttggttacgtctctgttgctttgcgaacttcctcatccagcgttcttgctgatccccgatattcagatgactcagaccaccagtagcttgtctgcctcactgggagaccgagtaacaatctcctgcagggcaagtcaagacattagcaaatacctcaattggtaccagcagaagcccgacggaacggtaaaactcctcatctatcatacgtcaaggttgcattccggagtaccgtcacgattttcaggttctgggagcggaactgactattccttgactatttcaaacctcgagcaggaggacattgcgacatatttttgtcaacaaggtaataccctcccttacactttcggaggaggaaccaaactcgaaattaccgggtccaccagtggctctgggaagcctggcagtggagaaggttccactaaaggcgaggtgaagctccaggagagcggccccggtctcgttgcccccagtcaaagcctctctgtaacgtgcacagtgagtggtgtatcattgcctgattatggcgtctcctggataaggcagcccccgcgaaagggtcttgaatggcttggggtaatatggggctcagagacaacgtattataactccgctctcaaaagtcgcttgacgataataaaagataactccaagagtcaagttttccttaaaatgaacagtttgcagactgacgataccgctatatattattgtgctaaacattattactacggcggtagttacgcgatggattattgggggcaggggacttctgtcacagtcagtagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 102 - protein, 484 aa, Artificial Sequence
  Synthetic
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - DNA, 735 bp, Artificial Sequence
  Synthetic
gatattcagatgactcagaccaccagtagcttgtctgcctcactgggagaccgagtaacaatctcctgcagggcaagtcaagacattagcaaatacctcaattggtaccagcagaagcccgacggaacggtaaaactcctcatctatcatacgtcaaggttgcattccggagtaccgtcacgattttcaggttctgggagcggaactgactattccttgactatttcaaacctcgagcaggaggacattgcgacatatttttgtcaacaaggtaataccctcccttacactttcggaggaggaaccaaactcgaaattaccgggtccaccagtggctctgggaagcctggcagtggagaaggttccactaaaggcgaggtgaagctccaggagagcggccccggtctcgttgcccccagtcaaagcctctctgtaacgtgcacagtgagtggtgtatcattgcctgattatggcgtctcctggataaggcagcccccgcgaaagggtcttgaatggcttggggtaatatggggctcagagacaacgtattataactccgctctcaaaagtcgcttgacgataataaaagataactccaagagtcaagttttccttaaaatgaacagtttgcagactgacgataccgctatatattattgtgctaaacattattactacggcggtagttacgcgatggattattgggggcaggggacttctgtcacagtcagtagt

SEQ ID NO: 104 - protein, 245 aa, Artificial Sequence
  Synthetic
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 105 - DNA, 261 bp, Artificial Sequence
  Synthetic
gctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgc

SEQ ID NO: 106 - DNA, 252 bp, Artificial Sequence
  Synthetic
tttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgc

SEQ ID NO: 107 - protein, 84 aa, Artificial Sequence
  Synthetic
FVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 108 - protein, 120 aa, Artificial Sequence
  Synthetic
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 109 - protein, 107 aa, Artificial Sequence
  Synthetic
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 110 - protein, 18 aa, Artificial Sequence
  Synthetic
GSTSGSGKPGSGEGSTKG

SEQ ID NO: 111 - protein, 15 aa, Artificial Sequence
  Synthetic
RASKSVSTSGYSFMH

SEQ ID NO: 112 - protein, 11 aa, Artificial Sequence
  Synthetic
SKSVSTSGYSF

SEQ ID NO: 113 - protein, 7 aa, Artificial Sequence
  Synthetic
LASNLES

SEQ ID NO: 114 - protein, 9 aa, Artificial Sequence
  Synthetic
QHSREVPWT

SEQ ID NO: 115 - protein, 6 aa, Artificial Sequence
  Synthetic
SREVPW

SEQ ID NO: 116 - protein, 5 aa, Artificial Sequence
  Synthetic
NYGMN

SEQ ID NO: 117 - protein, 10 aa, Artificial Sequence
  Synthetic
GYTFTNYGMN

SEQ ID NO: 118 - protein, 17 aa, Artificial Sequence
  Synthetic
WINTYTGEPTYADAFKG

SEQ ID NO: 119 - protein, 6 aa, Artificial Sequence
  Synthetic
NTYTGE

SEQ ID NO: 120 - protein, 9 aa, Artificial Sequence
  Synthetic
DYGDYGMDY

SEQ ID NO: 121 - protein, 14 aa, Artificial Sequence
  Synthetic
CARDYGDYGMDYWG

SEQ ID NO: 122 - DNA, 1527 bp, Artificial Sequence
  Synthetic
atggcgcttccggtgacagcactgctcctccccttggcgctgttgctccacgcagcaaggccgcaggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataa

SEQ ID NO: 123 - protein, 487 aa, Artificial Sequence
  Synthetic
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 124 - DNA, 735 bp, Artificial Sequence
  Synthetic
gatatagttatgacccaatcacccgatagtcttgcggtaagcctgggggagcgagcaacaataaactgtcgggcatcaaaatccgtcagtacaagcgggtattcattcatgcactggtatcaacagaaacccggtcagccacccaagctcctgatttatcttgcgtctaatcttgagtccggcgtcccagaccggttttccggctccgggagcggcacggattttactcttactatttctagccttcaggccgaagatgtggcggtatactactgccagcattcaagggaagttccttggacgttcggtcagggcacgaaagtggaaattaaaggcggggggggatccggcgggggagggtctggaggaggtggcagtggtcaggtccaactggtgcagtccggggcagaggtaaaaaaacccggcgcgtctgttaaggtttcatgcaaggccagtggatatactttcaccaattacggaatgaactgggtgaggcaggcccctggtcaaggcctgaaatggatgggatggataaacacgtacaccggtgaacctacctatgccgatgcctttaagggtcgggttacgatgacgagagacacctccatatcaacagcctacatggagctcagcagattgaggagtgacgatacggcagtctattactgtgcaagagactacggcgattatggcatggattactggggccagggcactacagtaaccgtttccagc

SEQ ID NO: 125 - protein, 245 aa, Artificial Sequence
  Synthetic
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKGGGGSGGGGSGGGGSGQVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 126 - DNA, 735 bp, Artificial Sequence
  Synthetic
caggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaa

SEQ ID NO: 127 - protein, 245 aa, Artificial Sequence
  Synthetic
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 128 - protein, 118 aa, Artificial Sequence
  Synthetic
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 129 - protein, 111 aa, Artificial Sequence
  Synthetic
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 130 - DNA, 1524 bp, Artificial Sequence
  Synthetic
atggcgcttccggtgacagcactgctcctccccttggcgctgttgctccacgcagcaaggccgcaggtgcagctggtgcagagcggagccgagctcaagaagcccggagcctccgtgaaggtgagctgcaaggccagcggcaacaccctgaccaactacgtgatccactgggtgagacaagcccccggccaaaggctggagtggatgggctacatcctgccctacaacgacctgaccaagtacagccagaagttccagggcagggtgaccatcaccagggataagagcgcctccaccgcctatatggagctgagcagcctgaggagcgaggacaccgctgtgtactactgtacaaggtgggactgggacggcttctttgacccctggggccagggcacaacagtgaccgtcagcagcggcggcggaggcagcggcggcggcggcagcggcggaggcggaagcgaaatcgtgatgacccagagccccgccacactgagcgtgagccctggcgagagggccagcatctcctgcagggctagccaaagcctggtgcacagcaacggcaacacccacctgcactggtaccagcagagacccggacaggctcccaggctgctgatctacagcgtgagcaacaggttctccgaggtgcctgccaggtttagcggcagcggaagcggcaccgactttaccctgaccatcagcagcgtggagtccgaggacttcgccgtgtattactgcagccagaccagccacatcccttacaccttcggcggcggcaccaagctggagatcaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 131 - protein, 487 aa, Artificial Sequence
  Synthetic
QVQLVQSGAELKKPGASVKVSCKASGNTLTNYVIHWVRQAPGQRLEWMGYILPYNDLTKYSQKFQGRVTITRDKSASTAYMELSSLRSEDTAVYYCTRWDWDGFFDPWGQGTTVTVSSGGGGSGGGGSGGGGSEIVMTQSPATLSVSPGERASISCRASQSLVHSNGNTHLHWYQQRPGQAPRLLIYSVSNRFSEVPARFSGSGSGTDFTLTISSVESEDFAVYYCSQTSHIPYTFGGGTKLEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - DNA, 735 bp, Artificial Sequence
  Synthetic
caggtgcagctggtgcagagcggagccgagctcaagaagcccggagcctccgtgaaggtgagctgcaaggccagcggcaacaccctgaccaactacgtgatccactgggtgagacaagcccccggccaaaggctggagtggatgggctacatcctgccctacaacgacctgaccaagtacagccagaagttccagggcagggtgaccatcaccagggataagagcgcctccaccgcctatatggagctgagcagcctgaggagcgaggacaccgctgtgtactactgtacaaggtgggactgggacggcttctttgacccctggggccagggcacaacagtgaccgtcagcagcggcggcggaggcagcggcggcggcggcagcggcggaggcggaagcgaaatcgtgatgacccagagccccgccacactgagcgtgagccctggcgagagggccagcatctcctgcagggctagccaaagcctggtgcacagcaacggcaacacccacctgcactggtaccagcagagacccggacaggctcccaggctgctgatctacagcgtgagcaacaggttctccgaggtgcctgccaggtttagcggcagcggaagcggcaccgactttaccctgaccatcagcagcgtggagtccgaggacttcgccgtgtattactgcagccagaccagccacatcccttacaccttcggcggcggcaccaagctggagatcaaa

SEQ ID NO: 133 - protein, 245 aa, Artificial Sequence
  Synthetic
QVQLVQSGAELKKPGASVKVSCKASGNTLTNYVIHWVRQAPGQRLEWMGYILPYNDLTKYSQKFQGRVTITRDKSASTAYMELSSLRSEDTAVYYCTRWDWDGFFDPWGQGTTVTVSSGGGGSGGGGSGGGGSEIVMTQSPATLSVSPGERASISCRASQSLVHSNGNTHLHWYQQRPGQAPRLLIYSVSNRFSEVPARFSGSGSGTDFTLTISSVESEDFAVYYCSQTSHIPYTFGGGTKLEIK

SEQ ID NO: 134 - protein, 118 aa, Artificial Sequence
  Synthetic
QVQLVQSGAELKKPGASVKVSCKASGNTLTNYVIHWVRQAPGQRLEWMGYILPYNDLTKYSQKFQGRVTITRDKSASTAYMELSSLRSEDTAVYYCTRWDWDGFFDPWGQGTTVTVSS

SEQ ID NO: 135 - protein, 112 aa, Artificial Sequence
  Synthetic
EIVMTQSPATLSVSPGERASISCRASQSLVHSNGNTHLHWYQQRPGQAPRLLIYSVSNRFSEVPARFSGSGSGTDFTLTISSVESEDFAVYYCSQTSHIPYTFGGGTKLEIK

SEQ ID NO: 136 - protein, 16 aa, Artificial Sequence
  Synthetic
RASQSLVHSNGNTHLH

SEQ ID NO: 137 - protein, 7 aa, Artificial Sequence
  Synthetic
SVSNRFS

SEQ ID NO: 138 - protein, 9 aa, Artificial Sequence
  Synthetic
SQTSHIPYT

SEQ ID NO: 139 - protein, 9 aa, Artificial Sequence
  Synthetic
SQTSHIPYT

SEQ ID NO: 140 - protein, 5 aa, Artificial Sequence
  Synthetic
NYVIH

SEQ ID NO: 141 - protein, 7 aa, Artificial Sequence
  Synthetic
GNTLTNY

SEQ ID NO: 142 - protein, 17 aa, Artificial Sequence
  Synthetic
YILPYNDLTKYSQKFQG

SEQ ID NO: 143 - protein, 6 aa, Artificial Sequence
  Synthetic
LPYNDL

SEQ ID NO: 144 - protein, 9 aa, Artificial Sequence
  Synthetic
WDWDGFFDP

SEQ ID NO: 145 - protein, 9 aa, Artificial Sequence
  Synthetic
WDWDGFFDP

SEQ ID NO: 146 - DNA, 145 bp, Artificial Sequence
  Synthetic
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 147 - DNA, 130 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 148 - DNA, 145 bp, Artificial Sequence
  Synthetic
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 149 - DNA, 141 bp, Artificial Sequence
  Synthetic
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 150 - DNA, 800 bp, Artificial Sequence
  Synthetic
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttca

SEQ ID NO: 151 - DNA, 804 bp, Artificial Sequence
  Synthetic
tggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 152 - DNA, 1178 bp, Artificial Sequence
  Synthetic
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 153 - DNA, 4358 bp, Artificial Sequence
  Synthetic
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatgcttcttttggttacgtctctgttgctttgcgaacttcctcatccagcgttcttgctgatccccgatattcagatgactcagaccaccagtagcttgtctgcctcactgggagaccgagtaacaatctcctgcagggcaagtcaagacattagcaaatacctcaattggtaccagcagaagcccgacggaacggtaaaactcctcatctatcatacgtcaaggttgcattccggagtaccgtcacgattttcaggttctgggagcggaactgactattccttgactatttcaaacctcgagcaggaggacattgcgacatatttttgtcaacaaggtaataccctcccttacactttcggaggaggaaccaaactcgaaattaccgggtccaccagtggctctgggaagcctggcagtggagaaggttccactaaaggcgaggtgaagctccaggagagcggccccggtctcgttgcccccagtcaaagcctctctgtaacgtgcacagtgagtggtgtatcattgcctgattatggcgtctcctggataaggcagcccccgcgaaagggtcttgaatggcttggggtaatatggggctcagagacaacgtattataactccgctctcaaaagtcgcttgacgataataaaagataactccaagagtcaagttttccttaaaatgaacagtttgcagactgacgataccgctatatattattgtgctaaacattattactacggcggtagttacgcgatggattattgggggcaggggacttctgtcacagtcagtagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 154 - DNA, 4364 bp, Artificial Sequence
  Synthetic
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 155 - DNA, 4364 bp, Artificial Sequence
  Synthetic
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggtgcagctggtgcagagcggagccgagctcaagaagcccggagcctccgtgaaggtgagctgcaaggccagcggcaacaccctgaccaactacgtgatccactgggtgagacaagcccccggccaaaggctggagtggatgggctacatcctgccctacaacgacctgaccaagtacagccagaagttccagggcagggtgaccatcaccagggataagagcgcctccaccgcctatatggagctgagcagcctgaggagcgaggacaccgctgtgtactactgtacaaggtgggactgggacggcttctttgacccctggggccagggcacaacagtgaccgtcagcagcggcggcggaggcagcggcggcggcggcagcggcggaggcggaagcgaaatcgtgatgacccagagccccgccacactgagcgtgagccctggcgagagggccagcatctcctgcagggctagccaaagcctggtgcacagcaacggcaacacccacctgcactggtaccagcagagacccggacaggctcccaggctgctgatctacagcgtgagcaacaggttctccgaggtgcctgccaggtttagcggcagcggaagcggcaccgactttaccctgaccatcagcagcgtggagtccgaggacttcgccgtgtattactgcagccagaccagccacatcccttacaccttcggcggcggcaccaagctggagatcaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 156 - protein, 506 aa, Artificial Sequence
  Synthetic
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 157 - protein, 508 aa, Artificial Sequence
  Synthetic
MALPVTALLLPLALLLHAARPQVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 158 - protein, 508 aa, Artificial Sequence
  Synthetic
MALPVTALLLPLALLLHAARPQVQLVQSGAELKKPGASVKVSCKASGNTLTNYVIHWVRQAPGQRLEWMGYILPYNDLTKYSQKFQGRVTITRDKSASTAYMELSSLRSEDTAVYYCTRWDWDGFFDPWGQGTTVTVSSGGGGSGGGGSGGGGSEIVMTQSPATLSVSPGERASISCRASQSLVHSNGNTHLHWYQQRPGQAPRLLIYSVSNRFSEVPARFSGSGSGTDFTLTISSVESEDFAVYYCSQTSHIPYTFGGGTKLEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2022/0193134 A1
(43) Pub. Date: Jun. 23, 2022

(54) CO-USE OF LENALIDOMIDE WITH CAR-T CELLS

(71) Applicant: CRISPR THERAPEUTICS AG, Zug (CH)

(72) Inventors: Henia DAR, Cambridge, MA (US); Jason SAGERT, Cambridge, MA (US); Jonathan Alexander TERRETT, Cambridge, MA (US); Hui YU, Cambridge, MA (US)

(21) Appl. No.: 17/559,355

(22) Filed: Dec. 22, 2021

Related U.S. Application Data
(60) Provisional application No. 63/129,967, filed on Dec. 23, 2020.

(51) Int. Cl. A61K 35/17; C12N 5/0783; A61K 31/454; C07K 16/28; A61P 35/00; C07K 14/705; C07K 14/725

(52) U.S. Cl. CPC A61K 35/17; C12N 5/0636; A61K 31/454; C07K 16/2878; A61P 35/00; C07K 2319/02; C07K 14/70521; C07K 14/7051; C12N 2501/999; C12N 2510/00; C07K 2317/622; C07K 14/70578

(57) ABSTRACT

A method for producing T cells expressing a chimeric antigen receptor (CAR-T cells), comprising: (i) culturing CAR-T cells in a medium comprising lenalidomide or a derivative thereof to produce CAR-T cells, and optionally (ii) administering the CAR-T cells to a subject in need of the treatment.

### CROSS REFERENCE TO RELATED APPLICATIONS

This application claims the benefit of the filing date of U.S. Provisional Application No. 63/129,967, filed Dec. 23, 2020, the entire contents of which are incorporated by reference herein.

### SEQUENCE LISTING

The application contains a Sequence Listing that has been filed electronically in the form of a text file, created Dec. 20, 2021, and named “095136-0488-036US1_SEQ.TXT” (116,881 bytes), the contents of which are incorporated by reference herein in their entirety.

### BACKGROUND OF THE INVENTION

Chimeric antigen receptor (CAR) T-cell therapy uses genetically-modified T cells to more specifically and efficiently target and kill cancer cells. After T cells have been collected from the blood, the cells are engineered to include CARs on their surface. The CARs may be introduced into the T cells using CRISPR/Cas9 gene editing technology. When these CAR T cells are injected into a patient, the receptors enable the T cells to kill cancer cells.

Lenalidomide and its derivatives thalidomide and pomalidomide are small molecule compounds that modulate the substrate activity of the CRL4^{CRBN} E3 ubiquitin ligase. These compounds are deemed as immunomodulatory drugs since they can increase IL-2 production in T lymphocytes and decrease pro-inflammatory cytokines. It is reported that lenalidomide can stimulate both T cells and NK cells, which could target both diseased cells and foreign cells.

### SUMMARY OF THE INVENTION

The present disclosure is based, at least in part, on the unexpected discovery that exposure of CAR-T cells to lenalidomide, either in vitro or in vivo, improved production and/or efficacy of the resultant CAR-T cells without enhancing immune recognition of allogenic CAR-T cells. Accordingly, provided herein are methods of producing CAR-T cells in the presence of lenalidomide or a derivative thereof and therapeutic applications of the CAR-T cells thus produced. Also provided herein are combined therapy comprising both CAR-T cells and lenalidomide or the derivative thereof.

In some aspects, the present disclosure provides a method for producing T cells expressing a chimeric antigen receptor (CAR-T cells), the method comprising: (i) culturing a first population of CAR-T cells in a medium comprising lenalidomide to produce a second population of CAR-T cells. Such a method may further comprise (ii) administering an effective amount of the second population of CAR-T cells produced in step (i) to a subject in need thereof. In some embodiments, the CAR-T cells are allogenic to the subject.

In other aspects, the present disclosure provides a method for improving treatment efficacy of T cells expressing a chimeric antigen receptor (CAR-T cells), the method comprising: administering an effective amount of CAR-T cells to a subject in need thereof, wherein the CAR-T cells have been cultured in vitro in the presence of lenalidomide or a derivative thereof. In some embodiments, the CAR-T cells are optionally allogeneic to the subject.

Further, provided herein is a method for eliminating undesired cells in a subject, the method comprising administering an effective amount of allogenic T cells expressing a chimeric antigen receptor (CAR-T cells) to a subject in need thereof, wherein the subject is undergoing a therapy comprising lenalidomide or a derivative thereof.

Also provided herein is a method for eliminating undesired cells in a subject, the method comprising (a) administering an effective amount of allogenic T cells expressing a chimeric antigen receptor (CAR-T cells) to a subject in need thereof, and (b) administering to the subject an effective amount of lenalidomide or a derivative thereof.

Moreover, the present disclosure features a method for eliminating undesired cells in a subject, the method comprising administering an effective amount of lenalidomide or a derivative thereof to a subject in need thereof, wherein the subject is undergoing a therapy comprising allogenic T cells expressing a chimeric antigen receptor (CAR-T cells).

In any of the methods disclosed herein, the CAR-T cells may be produced by an in vitro culturing process comprising lenalidomide or a derivative thereof.

The CAR-T cells produced in the presence of lenalidomide, either in vitro or in vivo, exhibit one or more of the following improved features as compared with the same CAR-T cells cultured in the absence of lenalidomide or a derivative thereof: (i) enhanced T cell proliferation and/or expansion capacity; (ii) increased T cell number; (iii) decreased senescence; (iv) improved effector activity, which optionally is characterized by improved cytokine secretion upon antigen stimulation; and/or improved cytotoxicity.

In any of the methods disclosed herein, the CAR comprises an extracellular antigen binding domain, which may be a single chain variable fragment (scFv), a co-stimulatory signaling domain of 4-1BB or CD28, and a cytoplasmic signaling domain of CD3ζ. The extracellular antigen binding domain may be specific to a tumor antigen. Exemplary tumor antigens include, but are not limited to, CD19, BCMA, and CD70.

In some embodiments, the CAR targets CD19. Such an anti-CD19 CAR may comprise an extracellular antigen binding domain, which can be a single chain variable fragment (scFv) that binds CD19. In some examples, anti-CD19 the scFv comprises the amino acid sequence of SEQ ID NO: 104. In specific examples, the anti-CD19 CAR comprises the amino acid sequence of SEQ ID NO: 102 (e.g., comprising the amino acid sequence of SEQ ID NO:156).

In some embodiments, the CAR targets BCMA. Such an anti-BCMA CAR may comprise an extracellular antigen binding domain, which can be a single chain variable fragment (scFv) that binds BCMA. In some examples, the anti-BCMA scFv comprises the amino acid sequence of SEQ ID NO: 133. In specific examples, the anti-BCMA CAR may comprise the amino acid sequence of SEQ ID NO: 131 (e.g., comprising the amino acid sequence of SEQ ID NO:157).

In some embodiments, the CAR targets CD70. Such an anti-CD70 CAR may comprise an extracellular antigen binding domain, which can be a single chain variable fragment (scFv) that binds CD70. In some instances, the scFv comprises the amino acid sequence of SEQ ID NO: 127. In specific examples, the anti-CD70 CAR may comprise the amino acid sequence of SEQ ID NO: 123 (e.g., comprising the amino acid sequence of SEQ ID NO:158).

In some embodiments, the nucleic acid encoding the CAR is inserted in a genomic site in the CAR-T cells. In some embodiments, the CAR-T cells have a disrupted TRAC gene, a disrupted β2M gene, or both. In some examples, the CAR-T cells have a disrupted TRAC gene, which comprises a deletion of a fragment having the nucleotide sequence of SEQ ID NO: 29. In some examples, the nucleic acid encoding the CAR is inserted in the disrupted TRAC gene. The nucleic acid encoding the CAR may substitutes for the fragment of SEQ ID NO: 29. In specific examples, the disrupted TRAC gene may comprise the nucleotide sequence of SEQ ID NO: 153, SEQ ID NO: 154, or SEQ ID NO: 155.

In some embodiments, the CAR-T cells comprise the disrupted TRAC gene and the disrupted β2M gene. In some examples, the disrupted β2M gene may comprise the nucleotide sequence of any one of SEQ ID NOs: 57 to 62.

In some embodiments, the CAR-T cells may further comprise a disrupted CD70 gene. In some instances, the disrupted CD70 gene comprises the nucleotide sequence of any one of SEQ ID NOs: 63-68. Such CAR-T cells may express an anti-CD70 CAR such as those disclosed herein.

Any of the CAR-T cells disclosed herein may further comprise a disrupted Regnase-1 (Reg1) gene, a disrupted TGFBRII gene, a disrupted TET2 gene, or a combination thereof. In some embodiments, the disrupted TRAC gene, the disrupted β2M gene, the disrupted CD70 gene, the disrupted Reg1 gene, and/or the disrupted TGFBRII gene are produced by a CRISPR/Cas gene editing system. For example, the disrupted TRAC gene can be targeted by an sgRNA comprising the nucleotide sequence of SEQ ID NO: 5, the disrupted B2M gene can be targeted by an sgRNA comprising the nucleotide sequence of SEQ ID NO: 9, the disrupted CD70 gene can be targeted by an sgRNA comprising the nucleotide sequence of SEQ ID NO: 13, the disrupted Reg1 gene can be targeted by an sgRNA comprising the nucleotide sequence of SEQ ID NO: 17, the disrupted TET gene can be targeted by an sgRNA comprising the nucleotide sequence of SEQ ID NO: 25; and/or the disrupted TGFBRII gene can be targeted by an sgRNA comprising the nucleotide sequence of SEQ ID NO:21.

In any of the methods disclosed herein, the subject is a human cancer patient. In some examples, the human patient has a CD19+ cancer. In some examples, the human patient has a BCMA+ cancer. In some examples, the patient has a CD70+ cancer.

Also within the scope of the present disclosure are CAR-T cells as those disclosed herein for treating cancer (e.g., cancers involving CD19⁺, BCMA⁺, or CD70⁺ cancer cells), wherein the CAR-T cells are exposed to lenalidomide or a derivative thereof in vitro, or uses of such CAR-T cells for manufacturing a medicament for use in treating the target cancer. Further, provided herein are co-uses of CAR-T cells as those disclosed herein and lenalidomide or a derivative thereof for the intended therapeutic purposes.

Further, the present disclosure provides a kit for use in cancer therapy, the kit comprising (i) a population of the CAR-T cells as disclosed herein, which may be produced by culturing in the presence of lenalidomide or a derivative thereof, and (ii) lenalidomide or a derivative thereof. Any of the CAR-T cells produced by in vitro exposure to lenalidomide or a derivative thereof, as well as medical uses thereof in cancer treatment, is also within the scope of the present disclosure.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A-1C are graphs showing that Lenalidomide (Len) addition demonstrates beneficial effect on multiple aspects of BCMA directed CAR-T cells in vitro. FIG. 1A is a graph showing that Lenalidomide enhances proliferation of BCMA directed CAR-T cells in vitro. FIG. 1B is a graph showing that Lenalidomide reduces the expression of a senescence marker in BCMA directed CAR-T cell in vitro. FIG. 1C includes graphs showing that Lenalidomide enhances secretion of effector cytokines following antigen stimulation of BCMA directed CAR-T cell in vitro.

FIGS. 2A-2C are graphs that show that Lenalidomide (Len) enhances BCMA directed CAR-T cell activity in vivo. FIG. 2A is a graph showing that combination of BCMA directed CAR-T cells & lenalidomide enhance tumor regression. Top panel: 1.5 mg/ml lenalidomide. Bottom panel: 10 mg/ml lenalidomide. FIG. 2B is a graph showing that combination of BCMA directed CAR-T cells & lenalidomide prolongs mouse survival. Upper panel: low dose of lenalidomide (1.5 mg/ml). Lower panel: high dose of lenalidomide (10 mg/ml). FIG. 2C is a graph showing that combination of BCMA directed CAR-T cells with lenalidomide enhances CAR-T expansion in mice.

FIGS. 3A-3C are graphs showing that Lenalidomide does not enhance immune recognition of allogenic T cells. FIG. 3A is a graph showing that Lenalidomide does not enhance NK cytotoxicity towards TRAC-/B2M-T cells. FIG. 3B includes graphs showing that Lenalidomide does not enhance secretion of cytokines by NK cells upon stimulation by Allo T cells. FIG. 3C are graphs that show that reduced allo reactivity towards TRAC-/B2M-allogenic T cells is maintained in the presence of Lenalidomide.

FIGS. 4A-4F includes graphs showing that BCMA directed CAR-T cells produced in the presence of Lenalidomide exhibit increased cytokine secretion upon antigen stimulation. FIG. 4A: IFNγ. FIG. 4B: IL-6. FIG. 4C: TNF-α. FIG. 4D: MCP-1. FIG. 4E: MIP1-α. FIG. 4F: MIP1-β.

FIGS. 5A-5K are graphs showing impact of Lenalidomide on CAR-T cell editing efficiency. FIGS. 5A and 5D are graphs showing the CAR+% of anti-CD19 CAR-T cells from two independent studies. FIGS. 5B and 5E are graphs showing the TRAC-% of anti-CD19 CAR-T cells from two independent studies. FIGS. 5C and 5F are graphs showing the B2M-% of anti-CD19 CAR-T cells from two independent studies. FIG. 5G is a graph showing the CAR+%, TRAC-%, and B2M-% of anti-BCMA CAR-T cells on day 8. FIG. 5H is a graph showing the CAR+% of anti-CD70 CAR-T cells from Process 1 and Process 2 on day 7 and day 14. From left to right, the bars represent Process 1 (underlined) at 0 μM and 10 μM and Process 2 at 0 μM, 0.5 μM, 1 μM, 2 μM, 5 μM, and 10 μM for each of Day 7 and Day 14. FIG. 5I is a graph showing the TRAC-% of anti-CD70 CAR-T cells (from Process 1 and Process 2) on day 7 and day 14. From left to right, the bars represent Process 1 (underlined) at 0 μM and 10 μM and Process 2 at 0 μM, 0.5 μM, 1 μM, 2 μM, 5 μM, and 10 μM for each of Day 7 and Day 14. FIG. 5J is a graph showing the B2M-% of anti-CD70 CAR-T cells (from Process 1 and Process 2) on day 7 and day 14. From left to right, the bars represent Process 1 (underlined) at 0 μM and 10 μM and Process 2 at 0 μM, 0.5 μM, 1 μM, 2 μM, 5 μM, and 10 μM for each of Day 7 and Day 14. FIG. 5K is a graph showing the CD70-% of anti-CD70 CAR-T cells (from Process 1 and Process 2) on day 7 and day 14. From left to right, the bars represent Process 1 (underlined), with or without CAR at 0 μM and 10 μM and Process 2 without CAR at 0 μM and 10 μM, or with CAR at 0 μM, 0.5 μM, 1 μM, 2 μM, 5 μM, and 10 μM for each of Day 7 and Day 14.

FIGS. 6A-6G are graphs showing impact of Lenalidomide on CAR-T cell CD4 and CD8 ratio. FIGS. 6A-6B show CD4% and CD8% of anti-CD19 CAR-T cells on day 6 and day 13. FIGS. 6C-6D show CD4% and CD8% from anti-CD19 CAR T cells on day 7 and day 15. FIG. 6E shows CD4% and CD8% from anti-BCMA CAR-T cells expanded at small and medium scale on day 8. FIGS. 6F-6G shows CD4% and CD8% from Anti-CD70 CAR-T cells (from Process 1 and Process 2) on day 7 and day 14. From left to right, the bars represent Process 1 (underlined) at 0 μM and 10 μM and Process 2 at 0 μM, 0.5 μM, 1 μM, 2 μM, 5 μM, and 10 μM for each of Day 7 and Day 14.

FIGS. 7A-7D are graphs that show the in vitro cytotoxicity of CAR-T cells cultured with Lenalidomide. FIGS. 7A-7B show anti-CD19 CAR-T cell cytotoxicity for varying effector CAR-T cell to target cell ratios on day 6 and day 13. FIGS. 7C-7D show anti-CD19 CAR-T cell cytotoxicity for varying effector CAR-T cell to target cell ratios on day 7 and day 15.

### DETAILED DESCRIPTION OF THE INVENTION

The present disclosure is based, at least in part, on the unexpected discovery that exposure of CAR-T cells to lenalidomide, either in vitro or in vivo, improved production and/or bioactivity of the resultant CAR-T cells without enhancing immune recognition of allogenic CAR-T cells.

In some instances, improved production may be reflected in enhanced T cell proliferation and/or expansion capacity relative to the same CAR-T cells having no exposure to lenalidomide or derivatives thereof. In some instances, improved production may be reflected in decreased senescence relative to the same CAR-T cells having no exposure to lenalidomide or derivatives thereof. In some instances, improved production may be reflected in a prolonged in vitro culture period during which the CAR-T cells maintains substantially the same growth activity and bioactivity, leading to increased T cell number of the therapeutic cell product thus produced, as relative to the same CAR-T cells having no exposure to lenalidomide or derivatives thereof.

Alternatively or in addition, the CAR-T cells produced by exposure to lenalidomide or a derivative thereof may exhibit enhanced bioactivity. In some instances, improved bioactivity may be reflected by improved cytotoxicity, either in vitro or in vivo, relative to the same CAR-T cells having no exposure to lenalidomide or derivatives thereof. In some instances, improved bioactivity may be reflected by improved effector activity, which can be characterized by improved cytokine secretion upon antigen stimulation relative to the same CAR-T cells having no exposure to lenalidomide or derivatives thereof. In some instances, improved bioactivity may be reflected by enhanced treatment efficacy (e.g., higher anti-cancer effect) as compared with the same CAR-T cells having no exposure to lenalidomide or derivatives thereof.

Thus, lenalidomide or its derivatives thereof can be co-used with CAR-T cells, either in vitro or in vivo, to improve production and/or bioactivity of the CAR-T cells. Accordingly, provided herein are methods for producing CAR-T cells in the presence of lenalidomide or a derivative thereof and methods of using the resultant CAR-T cells for treating diseases such as cancer. Also provided herein are combined therapy comprising CAR-T cells and lenalidomide. Given the impact of lenalidomide on CAR-T cells for enhancing production and/or bioactivity, such a combined therapy would be expected to lead to superior treatment efficacy.

### I. Genetically Engineered T Cells Expressing a Chimeric Antigen Receptor (CAR)

The genetically engineered T cells disclosed herein may express a chimeric antigen receptor (CAR) targeting an antigen of interest, and optionally one or more additional gene edits. The one or more additional gene edits may comprise disrupting genes for producing allogeneic T cells, e.g., a disrupted TRAC gene, in which the nucleic acid encoding the CAR may be inserted, a disrupted β2M gene, or a combination thereof.

The CAR-T cells disclosed herein may further comprise one or more additional gene edits (e.g., gene knock-in or knock-out) to improve T cell function. Examples include knock-in or knock-out genes to improve target cell lysis, knock-in or knock-out genes to enhance performance of therapeutic T cells such as CAR-T cells prepared from the genetically engineered T cells. For example, the CAR-T cells disclosed herein may further comprise disrupted genes for enhancing T cell features (e.g., enhanced growth and expansion capacity, enhanced T cell persistence, reduced T cell exhaustion, and/or resistance to inhibitory factors found at tumor microenvironment, etc.), for example, a disrupted TET2 gene, a disrupted Regnase-1 (Reg1) gene, a disrupted CD70 gene, a disrupted TGFBRII gene, or any combination thereof. Alternatively or in addition, the CAR-T cells disclosed herein may comprise a disrupted gene that encodes the antigen of interest, to which the CAR targets.

The genetically engineered T cells may be derived from parent T cells (e.g., non-edited wild-type T cells) obtained from a suitable source, for example, one or more mammal donors. In some examples, the parent T cells are primary T cells (e.g., non-transformed and terminally differentiated T cells) obtained from one or more human donors (e.g., healthy donors). Alternatively, the parent T cells may be differentiated from precursor T cells obtained from one or more suitable donor or stem cells such as hematopoietic stem cells or inducible pluripotent stem cells (iPSC), which may be cultured in vitro. In other examples, T cells from a T cell bank can be used as the starting material for preparing the genetically engineered T cells disclosed herein.

Any of the disrupted genes disclosed herein may be generated via gene editing (including genomic editing), a type of genetic engineering, in which nucleotide(s)/nucleic acid(s) is/are inserted, deleted, and/or substituted in a DNA sequence, such as in the genome of a targeted cell. Targeted gene editing enables insertion, deletion, and/or substitution at pre-selected sites in the genome of a targeted cell (e.g., in a targeted gene or targeted DNA sequence). When a sequence of an endogenous gene is edited, for example by deletion, insertion or substitution of nucleotide(s)/nucleic acid(s), the endogenous gene comprising the affected sequence may be knocked-out due to the sequence alteration. Therefore, targeted editing may be used to disrupt endogenous gene expression. “Targeted integration” refers to a process involving insertion of one or more exogenous sequences, with or without deletion of an endogenous sequence at the insertion site. Targeted integration can result from targeted gene editing when a donor template containing an exogenous sequence is present.

A. Genetically Edited Genes

In some aspects, the present disclosure provides CAR-T cells that comprise one or more disrupted genes as disclosed herein, for example, a disrupted TRAC gene, a disrupted β2M gene, a disrupted CD70 gene, a disrupted TET2 gene, a disrupted Reg1 gene, a disrupted TGFBRII gene, or a combination thereof.

As used herein, a “disrupted gene” refers to a gene comprising an insertion, deletion or substitution relative to an endogenous gene such that expression of a functional protein from the endogenous gene is reduced or inhibited. As used herein, “disrupting a gene” refers to a method of inserting, deleting or substituting at least one nucleotide/nucleic acid in an endogenous gene such that expression of a functional protein from the endogenous gene is reduced or inhibited. Methods of disrupting a gene are known to those of skill in the art and described herein.

In some embodiments, a cell that comprises a disrupted gene does not express (e.g., at the cell surface) a detectable level (e.g., in an immune assay using an antibody binding to the encoded protein or by flow cytometry) of the protein encoded by the gene. A cell that does not express a detectable level of the protein may be referred to as a knockout cell.

TRAC Gene Edit

In some embodiments, the genetically engineered T cells as disclosed herein may further comprise a disrupted TRAC gene. This disruption leads to loss of function of the TCR and renders the engineered T cell non-alloreactive and suitable for allogeneic transplantation, minimizing the risk of graft versus host disease. In some embodiments, expression of the endogenous TRAC gene is eliminated to prevent a graft-versus-host response. See also WO2019097305, the relevant disclosures of which are incorporated by reference herein for the purpose and subject matter referenced herein.

The disrupted TRAC gene may comprise one or more genetic edits at one or more suitable target sites (e.g., in coding regions or in non-coding regulatory regions such as promoter regions) that disrupt expression of the TRAC gene. Such target sites may be identified based on the gene editing approach for use in making the genetically engineered T cells. In some examples, the target sites for the genetic edits may be in exon 1 of the TRAC gene. Such genetic editing may be induced by a gene editing technology, (e.g., the CRISPR/Cas technology) using a suitable guide RNA, for example, those listed in Table 1. In some embodiments, an edited TRAC gene may comprise a nucleotide sequence selected from the sequences in Table 2. It should be understood that more than one suitable target site/gRNA can be used for each target gene disclosed herein, for example, those known in the art or disclosed herein. Additional examples can be found in, e.g., WO2019097305, the relevant disclosures of which are incorporated by reference herein for the purpose and subject matter referenced herein.

In some examples, a nucleic acid encoding the CAR as disclosed herein may be inserted into the disrupted TRAC gene locus, for example, at the target site for the genetic editing. In some examples, the disrupted TRAC gene may comprise a deletion of a fragment comprising SEQ ID NO: 29. In specific examples, the fragment comprising SEQ ID NO:29 may be replaced by the nucleic acid coding for the CAR.

β2M Gene Edit

In some embodiments, the genetically engineered T cells disclosed herein may further comprise a disrupted β2M gene. β2M is a common (invariant) component of MHC I complexes. Disrupting its expression by gene editing will prevent host versus therapeutic allogeneic T cells responses leading to increased allogeneic T cell persistence. In some embodiments, expression of the endogenous β2M gene is eliminated to prevent a host-versus-graft response.

In some embodiments, an edited β2M gene may comprise a nucleotide sequence selected from the following sequences in Table 3. It is known to those skilled in the art that different nucleotide sequences in an edited gene such as an edited β2M gene (e.g., those in Table 3) may be generated by a single gRNA. See also WO2019097305, the relevant disclosures of which are incorporated by reference for the subject matter and purpose referenced herein. Such genetic editing may be induced by a gene editing technology, (e.g., the CRISPR/Cas technology) using a suitable guide RNA, for example, those listed in Table 1.

CD70 Gene Editing

T cell exhaustion is a process of stepwise and progressive loss of T cell functions, which may be induced by prolonged antigen stimulation or other factors. Genes involved in T cell exhaustion refer to those that either positively regulate or negatively regulate this biological process. The genetically engineered T cells disclosed herein may comprise genetic editing of a gene that positively regulates T cell exhaustion to disrupt its expression. Alternatively or in addition, the genetically engineered T cells may comprise genetic editing of a gene that negatively regulates T cell exhaustion to enhance its expression and/or biologic activity of the gene product.

In some embodiments, the CAR-T cells may comprise an edited gene involved in T cell exhaustion, e.g., disruption of a gene that positively regulates T cell exhaustion. Such a gene may be a Cluster of Differentiation 70 (CD70) gene. CD70 is a member of the tumor necrosis factor superfamily and its expression is restricted to activated T and B lymphocytes and mature dendritic cells. CD70 is implicated in tumor cell and regulatory T cell survival through interaction with its ligand, CD27. CD70 and its receptor CD27 have multiple roles in immune function in multiple cell types including T cells (activated and T_{reg} cells), and B cells. In some embodiments, an edited CD70 gene may comprise a nucleotide sequence selected from the following sequences in Table 4. Such genetic editing may be induced by a gene editing technology, (e.g., the CRISPR/Cas technology) using a suitable guide RNA, for example, those listed in Table 1.

It was also found that disrupting the CD70 gene in immune cells engineered to express an antigen targeting moiety enhanced anti-tumor efficacy against large tumors and induced a durable anti-cancer memory response. Specifically, the anti-cancer memory response prevented tumor growth upon re-challenge. Further, it has been demonstrated disrupting the CD70 gene results in enhanced cytotoxicity of immune cells engineered to express an antigen targeting moiety at lower ratios of engineered immune cells to target cells, indicating the potential efficacy of low doses of engineered immune cells. See, e.g., WO2019/215500, the relevant disclosures of which are incorporated by reference for the purpose and subject matter referenced herein.

Structures of CD70 genes are known in the art. For example, human CD70 gene is located on chromosome 19p13.3. The gene contains four protein encoding exons. Additional information can be found in GenBank under Gene ID: 970.

In some examples, the CAR-T cells may comprise a disrupted CD70 gene such that the expression of CD70 in the T cells is substantially reduced or eliminated completely. The disrupted CD70 gene may comprise one or more genetic edits at one or more suitable target sites (e.g., in coding regions or in non-coding regulatory regions such as promoter regions) that disrupt expression of the CD70 gene. Such target sites may be identified based on the gene editing approach for use in making the genetically engineered T cells. Exemplary target sites for the genetic edits may include exon 1, exon 2, exon 3, exon 4, or a combination thereof. See also WO2019/215500, the relevant disclosures of which are incorporated by reference for the purpose and subject matter referenced herein.

TGFBRII Gene Editing

In some embodiments, the CAR-T cells disclosed herein may comprise a disrupted TGFBRII gene, which encodes Transforming Growth Factor Receptor Type II (TGFBRII). TGFBRII receptors are a family of serine/threonine kinase receptors involved in the TGFβ signaling pathway. These receptors bind growth factor and cytokine signaling proteins in the TGFβ family, for example, TGFβs (TGFβ1, TGFβ2, and TGFβ3), bone morphogenetic proteins (BMPs), growth differentiation factors (GDFs), activin and inhibin, myostatin, anti-Müllerian hormone (AMH), and NODAL.

In some examples, the genetically engineered T cells may comprise a disrupted TGFBRII gene such that the expression of TGFBRII in the T cells is substantially reduced or eliminated completely. The disrupted TGFBRII gene may comprise one or more genetic edits at one or more suitable target sites (e.g., in coding regions or in non-coding regulatory regions such as promoter regions) that disrupt expression of the TGFBRII gene. Such target sites may be identified based on the gene editing approach for use in making the genetically engineered T cells. Exemplary target sites for the genetic edits may include exon 1, exon 2, exon 3, exon 4, exon 5, or a combination thereof. In some examples, one or more genetic editing may occur in exon 4. Such genetic editing may be induced by a gene editing technology, (e.g., the CRISPR/Cas technology) using a suitable guide RNA, for example, those listed in Table 1.

Regnase-1 (Reg1) Gene Edit

In some embodiments, the CAR-T cells may comprise a disrupted gene involved in mRNA decay. Such a gene may be Reg1. Reg1 contains a zinc finger motif, binds RNA and exhibits ribonuclease activity. Reg1 plays roles in both immune and non-immune cells and its expression can be rapidly induced under diverse conditions including microbial infections, treatment with inflammatory cytokines and chemical or mechanical stimulation. Human Reg1 gene is located on chromosome 1p34.3. Additional information can be found in GenBank under Gene ID: 80149.

In some examples, the genetically engineered T cells may comprise a disrupted Reg1 gene such that the expression of Reg1 in the T cells is substantially reduced or eliminated completely. The disrupted Reg1 gene may comprise one or more genetic edits at one or more suitable target sites (e.g., in coding regions or in non-coding regulatory regions such as promoter regions) that disrupt expression of the Reg1 gene. Such target sites may be identified based on the gene editing approach for use in making the genetically engineered T cells. Exemplary target sites for the genetic edits may include exon 1, exon 2, exon 3, exon 4, exon 5, exon 6, or a combination thereof. In some examples, one or more genetic editing may occur in exon 2 or exon 4. Such genetic editing may be induced by the CRISPR/Cas technology using a suitable guide RNA, for example, those listed in Table 1. The resultant edited Reg1 gene using a gRNA listed in Table 1 may comprise one or more edited sequences provided in Table 5 below.

Tet Methylcytosine Dioxygenase 2 Gene (TET2) Edit

Self-renewal is the process by which cells (e.g., T cells) divide and maintain the same cell state/identity. Genes involved in cell self-renewal refer to those that either positively regulate or negatively regulate cell self-renewal. The genetically engineered T cells disclosed herein may comprise genetic editing of a gene that positively regulates cell self-renewal to enhance its expression and/or bioactivity of the encoded protein product. Alternatively or in addition, the genetically engineered T cells may comprise genetic editing of a gene that negatively regulates cell self-renewal to disrupt its expression.

In some embodiments, the CAR-T cells disclosed herein may comprise a mutated gene involved in cell self-renewal. Such a gene may be TET2, which encodes a Methylcytosine Dioxygenase. Tet2 is a dioxygenase that catalyzes the conversion of the modified genomic base methylcytosine to 5-hydroxymethylcytosine and to further intermediates leading to cytosine demethylation. This enzyme is involved in myelopoiesis, and defects in TET2 have been reported to be associated with several myeloproliferative disorders. Structures of TET2 genes are known in the art. For example, human TET2 gene is located on chromosome 4q24. Additional information can be found in GenBank under Gene ID: 54790 or NCBI Reference Sequence: NM_001127208.2.

In some examples, the genetically engineered T cells may comprise a disrupted TET2 gene such that the expression of TET2 in the T cells is substantially reduced or eliminated completely. The disrupted TET2 gene may comprise one or more genetic edits at one or more suitable target sites (e.g., in coding regions or in non-coding regulatory regions such as promoter regions) that disrupt expression of the TET2 gene. Such target sites may be identified based on the gene editing approach for use in making the genetically engineered T cells. Exemplary target sites for the genetic edits may include exon 1, exon 3, exon 4, exon 5, exon 6, or a combination thereof. In some examples, one or more genetic edits may occur in exon 3, exon 4, exon 5, or exon 6. Such genetic editing may be induced by the CRISPR/Cas technology using a suitable guide RNA, for example, those listed in Table 1. The resultant edited TET2 gene using a gRNA listed in Table 1 may comprise one or more edited sequences provided in Table 6 below.

B. Methods of Making Gene Editing in T Cells

The CAR-T cells disclosed herein can be prepared by genetic editing of parent T cells or precursor cells thereof via a conventional gene editing method or those described herein.

(a) T Cells

In some embodiments, T cells for generating the genetically engineered T cells disclosed herein can be derived from one or more suitable mammals, for example, one or more human donors. T cells can be obtained from a number of sources, including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, T cells can be obtained from a unit of blood collected from a subject using any number of techniques known to the skilled person, such as sedimentation, e.g., FICOLL™ separation. In some instances, the T cell population comprises primary T cells isolated from one or more human donors. Such T cells are terminally differentiated, not transformed, depend on cytokines and/or growth factors for growth, and/or have stable genomes.

In some examples, T cells can be isolated from a mixture of immune cells (e.g., those described herein) to produce an isolated T cell population. For example, after isolation of peripheral blood mononuclear cells (PBMC), both cytotoxic and helper T lymphocytes can be sorted into naive, memory, and effector T cell subpopulations either before or after activation, expansion, and/or genetic modification.

A specific subpopulation of T cells, expressing one or more of the following cell surface markers: TCRαβ, CD3, CD4, CD8, CD27 CD28, CD38 CD45RA, CD45RO, CD62L, CD127, CD122, CD95, CD197, CCR7, KLRG1, MCH-I proteins and/or MCH-II proteins, can be further isolated by positive or negative selection techniques. In some embodiments, a specific subpopulation of T cells, expressing one or more of the markers selected from the group consisting of TCRαβ, CD4 and/or CD8, is further isolated by positive or negative selection techniques. In some embodiments, subpopulations of T cells may be isolated by positive or negative selection prior to genetic engineering and/or post genetic engineering.

An isolated population of T cells may express one or more of the T cell markers, including, but not limited to a CD3+, CD4+, CD8+, or a combination thereof. In some embodiments, the T cells are isolated from a donor, or subject, and first activated and stimulated to proliferate in vitro prior to undergoing gene editing.

In other embodiments, the T cells for use in generating the genetically engineered T cells disclosed herein may be derived from a T cell bank. A T cell bank may comprise T cells with genetic editing of certain genes (e.g., genes involved in cell self renewal, apoptosis, and/or T cell exhaustion or replicative senescence) to improve T cell persistence in cell culture. A T cell bank may be produced from bona fide T cells, for example, non-transformed T cells, terminally differentiated T cells, T cells having stable genome, and/or T cells that depend on cytokines and growth factors for proliferation and expansion. Alternatively, such a T cell bank may be produced from precursor cells such as hematopoietic stem cells (e.g., iPSCs), e.g., in vitro culture. In some examples, the T cells in the T cell bank may comprise genetic editing of one or more genes involved in cell self-renewal, one or more genes involved in apoptosis, and/or one or more genes involved in T cell exhaustion, so as to disrupt or reduce expression of such genes, leading to improved persistence in culture. Examples of the edited genes in a T cell bank include, but are not limited to, Tet2, Fas, CD70, Reg1, or a combination thereof. Compared with the non-edited T counterpart, T cells in a T cell bank may have enhanced expansion capacity in culture, enhanced proliferation capacity, greater T cell activation, and/or reduced apoptosis levels. Additional information of T cell bank may be found in International Application No. PCT/IB2020/058280, the relevant disclosures of which are incorporated by reference for the subject matter and purpose referenced herein.

In yet other embodiments, the T cells for generating the genetically engineered T cells disclosed herein may be derived from stem cells (e.g., HSCs or iPSCs) via in vitro differentiation.

T cells from any suitable source (e.g., those disclosed herein) can be subjected to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041. In some embodiments, T cells can be activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days prior to introduction of the genome editing compositions into the T cells.

In some embodiments, T cells can be activated and expanded for about 4 hours, about 6 hours, about 12 hours, about 18 hours, about 24 hours, about 36 hours, about 48 hours, about 60 hours, or about 72 hours prior to introduction of the gene editing compositions into the T cells. In some embodiments, T cells are activated at the same time that genome editing compositions are introduced into the T cells. In some instances, the T cell population can be expanded and/or activated after the genetic editing as disclosed herein. T cell populations or isolated T cells generated by any of the gene editing methods described herein are also within the scope of the present disclosure.

(b) Gene Editing Methods

Any of the genetically engineered T cells can be prepared using conventional gene editing methods or those described herein to edit one or more of the target genes disclosed herein (targeted editing). Targeted editing can be achieved either through a nuclease-independent approach, or through a nuclease-dependent approach. In the nuclease-independent targeted editing approach, homologous recombination is guided by homologous sequences flanking an exogenous polynucleotide to be introduced into an endogenous sequence through the enzymatic machinery of the host cell. The exogenous polynucleotide may introduce deletions, insertions or replacement of nucleotides in the endogenous sequence.

Alternatively, the nuclease-dependent approach can achieve targeted editing with higher frequency through the specific introduction of double strand breaks (DSBs) by specific rare-cutting nucleases (e.g., endonucleases). Such nuclease-dependent targeted editing also utilizes DNA repair mechanisms, for example, non-homologous end joining (NHEJ), which occurs in response to DSBs. DNA repair by NHEJ often leads to random insertions or deletions (indels) of a small number of endogenous nucleotides. In contrast to NHEJ mediated repair, repair can also occur by a homology directed repair (HDR). When a donor template containing exogenous genetic material flanked by a pair of homology arms is present, the exogenous genetic material can be introduced into the genome by HDR, which results in targeted integration of the exogenous genetic material.

In some embodiments, gene disruption may occur by deletion of a genomic sequence using two guide RNAs. Methods of using CRISPR-Cas gene editing technology to create a genomic deletion in a cell (e.g., to knock out a gene in a cell) are known (Bauer D E et al. Vis. Exp. 2015; 95:e52118).

Available endonucleases capable of introducing specific and targeted DSBs include, but not limited to, zinc-finger nucleases (ZFN), transcription activator-like effector nucleases (TALEN), and RNA-guided CRISPR-Cas9 nuclease (CRISPR/Cas9; Clustered Regular Interspaced Short Palindromic Repeats Associated 9). Additionally, DICE (dual integrase cassette exchange) system utilizing phiC31 and Bxb1 integrases may also be used for targeted integration. Some exemplary approaches are disclosed in detail below.

CRISPR-Cas9 Gene Editing System

The CRISPR-Cas9 system is a naturally-occurring defense mechanism in prokaryotes that has been repurposed as an RNA-guided DNA-targeting platform used for gene editing. It relies on the DNA nuclease Cas9, and two noncoding RNAs, crisprRNA (crRNA) and trans-activating RNA (tracrRNA), to target the cleavage of DNA. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see, e.g., Koonin et al., (2017) Curr Opin Microbiol 37:67-78).

crRNA drives sequence recognition and specificity of the CRISPR-Cas9 complex through Watson-Crick base pairing typically with a 20 nucleotide (nt) sequence in the target DNA. Changing the sequence of the 5′ 20 nt in the crRNA allows targeting of the CRISPR-Cas9 complex to specific loci. The CRISPR-Cas9 complex only binds DNA sequences that contain a sequence match to the first 20 nt of the crRNA, if the target sequence is followed by a specific short DNA motif (with the sequence NGG) referred to as a protospacer adjacent motif (PAM). tracrRNA hybridizes with the 3′ end of crRNA to form an RNA-duplex structure that is bound by the Cas9 endonuclease to form the catalytically active CRISPR-Cas9 complex, which can then cleave the target DNA.

Once the CRISPR-Cas9 complex is bound to DNA at a target site, two independent nuclease domains within the Cas9 enzyme each cleave one of the DNA strands upstream of the PAM site, leaving a double-strand break (DSB), where both strands of the DNA terminate in a base pair (a blunt end). After binding of CRISPR-Cas9 complex to DNA at a specific target site and formation of the site-specific DSB, the next key step is repair of the DSB. Cells use two main DNA repair pathways to repair the DSB: non-homologous end joining (NHEJ) and homology-directed repair (HDR).

NHEJ is a robust repair mechanism that appears highly active in the majority of cell types, including non-dividing cells. NHEJ is error-prone and can often result in the removal or addition of between one and several hundred nucleotides at the site of the DSB, though such modifications are typically <20 nt. The resulting insertions and deletions (indels) can disrupt coding or noncoding regions of genes. Alternatively, HDR uses a long stretch of homologous donor DNA, provided endogenously or exogenously, to repair the DSB with high fidelity. HDR is active only in dividing cells and occurs at a relatively low frequency in most cell types. In many embodiments of the present disclosure, NHEJ is utilized as the repair operant.

Endonuclease for Use in CRISPR

In some embodiments, the Cas9 (CRISPR associated protein 9) endonuclease is used in a CRISPR method for making the genetically engineered T cells as disclosed herein. The Cas9 enzyme may be one from Streptococcus pyogenes, although other Cas9 homologs may also be used. It should be understood, that wild-type Cas9 may be used or modified versions of Cas9 may be used (e.g., evolved versions of Cas9, or Cas9 orthologues or variants), as provided herein. In some embodiments, Cas9 may be substituted with another RNA-guided endonuclease, such as Cpf1 (of a class II CRISPR/Cas system).

In some embodiments, the CRISPR/Cas system comprises components derived from a Type-I, Type-II, or Type-III system. Updated classification schemes for CRISPR/Cas loci define Class 1 and Class 2 CRISPR/Cas systems, having Types I to V or VI (Makarova et al., (2015) Nat Rev Microbiol, 13(11):722-36; Shmakov et al., (2015) Mol Cell, 60:385-397). Class 2 CRISPR/Cas systems have single protein effectors. Cas proteins of Types II, V, and VI are single-protein, RNA-guided endonucleases, herein called “Class 2 Cas nucleases.” Class 2 Cas nucleases include, for example, Cas9, Cpf1, C2c1, C2c2, and C2c3 proteins. The Cpf1 nuclease (Zetsche et al., (2015) Cell 163:1-13) is homologous to Cas9 and contains a RuvC-like nuclease domain.

In some embodiments, the Cas nuclease is from a Type-II CRISPR/Cas system (e.g., a Cas9 protein from a CRISPR/Cas9 system). In some embodiments, the Cas nuclease is from a Class 2 CRISPR/Cas system (a single-protein Cas nuclease such as a Cas9 protein or a Cpf1 protein). The Cas9 and Cpf1 family of proteins are enzymes with DNA endonuclease activity, and they can be directed to cleave a desired nucleic acid target by designing an appropriate guide RNA, as described further herein.

In some embodiments, a Cas nuclease may comprise more than one nuclease domain. For example, a Cas9 nuclease may comprise at least one RuvC-like nuclease domain (e.g., Cpf1) and at least one HNH-like nuclease domain (e.g., Cas9). In some embodiments, the Cas9 nuclease introduces a DSB in the target sequence. In some embodiments, the Cas9 nuclease is modified to contain only one functional nuclease domain. For example, the Cas9 nuclease is modified such that one of the nuclease domains is mutated or fully or partially deleted to reduce its nucleic acid cleavage activity. In some embodiments, the Cas9 nuclease is modified to contain no functional RuvC-like nuclease domain. In other embodiments, the Cas9 nuclease is modified to contain no functional HNH-like nuclease domain. In some embodiments in which only one of the nuclease domains is functional, the Cas9 nuclease is a nickase that is capable of introducing a single-stranded break (a “nick”) into the target sequence. In some embodiments, a conserved amino acid within a Cas9 nuclease domain is substituted to reduce or alter a nuclease activity. In some embodiments, the Cas nuclease nickase comprises an amino acid substitution in the RuvC-like nuclease domain. Exemplary amino acid substitutions in the RuvC-like nuclease domain include D10A (based on the S. pyogenes Cas9 nuclease). In some embodiments, the nickase comprises an amino acid substitution in the HNH-like nuclease domain. Exemplary amino acid substitutions in the HNH-like nuclease domain include E762A, H840A, N863A, H983A, and D986A (based on the S. pyogenes Cas9 nuclease).

Amino acid sequence of Cas9 nuclease (SEQ ID NO: 1):
MDKKYSIGLDIGTNSVGWAVITDEYKVPSKKEKVLGNTDRHSIKKNLIGALL
EDSGETAEATRLKRTARRRYTRRKNRICYLQEIFSNEMAKVDDSFEHRLEES
ELVEEDKKHERHPIFGNIVDEVAYHEKYPTIYHLRKKLVDSTDKADLRLIYL
ALAHMIKFRGHFLIEGDLNPDNSDVDKLFIQLVQTYNQLFEENPINASGVDA
KAILSARLSKSRRLENLIAQLPGEKKNGLFGNLIALSLGLIPNEKSNEDLAE
DAKLQLSKDTYDDDLDNLLAQIGDQYADLFLAAKNLSDAILLSDILRVNTEI
TKAPLSASMIKRYDEHHQDLTLLKALVRQQLPEKYKEIFFDQSKNGYAGYID
GGASQEEFYKFIKPILEKMDGTEELLVKLNREDLLRKQRTFDNGSIPHQIHL
GELHAILRRQEDFYPFLKDNREKIEKILTFRIPYYVGPLARGNSRFAWMTRK
SEETITPWNFEEVVDKGASAQSFIERMTNEDKNLPNEKVLPKHSLLYEYFTV
YNELTKVKYVTEGMRKPAELSGEQKKAIVDLLFKINRKVIVKQLKEDYFKKI
ECFDSVEISGVEDRFNASLGTYHDLLKIIKDKDFLDNEENEDILEDIVLTLT
LFEDREMIEERLKTYAHLFDDKVMKQLKRRRYTGWGRLSRKLINGIRDKQSG
KTILDFLKSDGFANRNFMQLIHDDSLTEKEDIQKAQVSGQGDSLHEHIANLA
GSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINR
LSDYDVDHIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILD
SRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYS
NIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLII
KLPKYSLFELENGRKRMLASAGELQKGNELALPSKYVNFLYLASHYEKLKGS
PEDNEQKQLFVEQHKHYLDEIIEQISEFSKRVILADANLDKVLSAYNKHRDK
PIREQAENIIHLETLTNLGAPAAFKYFDTTIDRKRYTSTKEVLDATLIHQSI
TGLYETRIDLSQLGGD

In some embodiments, the Cas nuclease is from a Type-I CRISPR/Cas system. In some embodiments, the Cas nuclease is a component of the Cascade complex of a Type-I CRISPR/Cas system. For example, the Cas nuclease is a Cas3 nuclease. In some embodiments, the Cas nuclease is derived from a Type-III CRISPR/Cas system. In some embodiments, the Cas nuclease is derived from Type-IV CRISPR/Cas system. In some embodiments, the Cas nuclease is derived from a Type-V CRISPR/Cas system. In some embodiments, the Cas nuclease is derived from a Type-VI CRISPR/Cas system.

Guide RNAs (gRNAs)

The CRISPR technology involves the use of a genome-targeting nucleic acid that can direct the endonuclease to a specific target sequence within a target gene for gene editing at the specific target sequence. The genome-targeting nucleic acid can be an RNA. A genome-targeting RNA is referred to as a “guide RNA” or “gRNA” herein. A guide RNA comprises at least a spacer sequence that hybridizes to a target nucleic acid sequence within a target gene for editing, and a CRISPR repeat sequence.

In Type II systems, the gRNA also comprises a second RNA called the tracrRNA sequence. In the Type II gRNA, the CRISPR repeat sequence and tracrRNA sequence hybridize to each other to form a duplex. In the Type V gRNA, the crRNA forms a duplex. In both systems, the duplex binds a site-directed polypeptide, such that the guide RNA and site-direct polypeptide form a complex. In some embodiments, the genome-targeting nucleic acid provides target specificity to the complex by virtue of its association with the site-directed polypeptide. The genome-targeting nucleic acid thus directs the activity of the site-directed polypeptide.

As is understood by the person of ordinary skill in the art, each guide RNA is designed to include a spacer sequence complementary to its genomic target sequence. See Jinek et al., Science, 337, 816-821 (2012) and Deltcheva et al., Nature, 471, 602-607 (2011).

In some embodiments, the genome-targeting nucleic acid (e.g., gRNA) is a double-molecule guide RNA. A double-molecule guide RNA comprises two strands of RNA molecules. The first strand comprises in the 5′ to 3′ direction, an optional spacer extension sequence, a spacer sequence and a minimum CRISPR repeat sequence. The second strand comprises a minimum tracrRNA sequence (complementary to the minimum CRISPR repeat sequence), a 3′ tracrRNA sequence and an optional tracrRNA extension sequence.

In some embodiments, the genome-targeting nucleic acid (e.g., gRNA) is a single-molecule guide RNA. A single-molecule guide RNA (referred to as a “sgRNA”) in a Type II system comprises, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-molecule guide linker links the minimum CRISPR repeat and the minimum tracrRNA sequence to form a hairpin structure. The optional tracrRNA extension comprises one or more hairpins. A single-molecule guide RNA in a Type V system comprises, in the 5′ to 3′ direction, a minimum CRISPR repeat sequence and a spacer sequence.

A spacer sequence in a gRNA is a sequence (e.g., a 20 nucleotide sequence) that defines the target sequence (e.g., a DNA target sequences, such as a genomic target sequence) of a target gene of interest. In some embodiments, the spacer sequence range from 15 to 30 nucleotides. For example, the spacer sequence may contain 15, 16, 17, 18, 19, 29, 21, 22, 23, 24, 25, 26, 27, 28, 29, or 30 nucleotides. In some embodiments, a spacer sequence contains 20 nucleotides.

The “target sequence” is in a target gene that is adjacent to a PAM sequence and is the sequence to be modified by an RNA-guided nuclease (e.g., Cas9). The “target sequence” is on the so-called PAM-strand in a “target nucleic acid,” which is a double-stranded molecule containing the PAM-strand and a complementary non-PAM strand. One of skill in the art recognizes that the gRNA spacer sequence hybridizes to the complementary sequence located in the non-PAM strand of the target nucleic acid of interest. Thus, the gRNA spacer sequence is the RNA equivalent of the target sequence. For example, if the target sequence is 5′-AGAGCAACAGTGCTGTGGCC**-3′ (SEQ ID NO: 29), then the gRNA spacer sequence is 5′-AGAGCAACAGUGCUGUGGCC**-3′ (SEQ ID NO: 5). The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

In a CRISPR/Cas system herein, the spacer sequence is designed to hybridize to a region of the target nucleic acid that is located 5′ of a PAM recognizable by a Cas9 enzyme used in the system. The spacer may perfectly match the target sequence or may have mismatches. Each Cas9 enzyme has a particular PAM sequence that it recognizes in a target DNA. For example, S. pyogenes recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence has 20 nucleotides in length. In some embodiments, the target nucleic acid has less than 20 nucleotides in length. In some embodiments, the target nucleic acid has more than 20 nucleotides in length. In some embodiments, the target nucleic acid has at least: 5, 10, 15, 16, 17, 18, 19, 20, 21, 22, 23, 24, 25, 30 or more nucleotides in length. In some embodiments, the target nucleic acid has at most: 5, 10, 15, 16, 17, 18, 19, 20, 21, 22, 23, 24, 25, 30 or more nucleotides in length. In some embodiments, the target nucleic acid sequence has 20 bases immediately 5′ of the first nucleotide of the PAM. For example, in a sequence comprising 5′-NNNNNNNNNNNNNNNNNNNNNRG-3′, the target nucleic acid can be the sequence that corresponds to the Ns, wherein N can be any nucleotide, and the underlined NRG sequence is the S. pyogenes PAM.

The guide RNA disclosed herein may target any sequence of interest via the spacer sequence in the crRNA. In some embodiments, the degree of complementarity between the spacer sequence of the guide RNA and the target sequence in the target gene can be about 60%, 65%, 70%, 75%, 80%, 85%, 90%, 95%, 97%, 98%, 99%, or 100%. In some embodiments, the spacer sequence of the guide RNA and the target sequence in the target gene is 100% complementary. In other embodiments, the spacer sequence of the guide RNA and the target sequence in the target gene may contain up to 10 mismatches, e.g., up to 9, up to 8, up to 7, up to 6, up to 5, up to 4, up to 3, up to 2, or up to 1 mismatch.

For any of the gRNA sequences provided herein, those that do not explicitly indicate modifications are meant to encompass both unmodified sequences and sequences having any suitable modifications.

The length of the spacer sequence in any of the gRNAs disclosed herein may depend on the CRISPR/Cas9 system and components used for editing any of the target genes also disclosed herein. For example, different Cas9 proteins from different bacterial species have varying optimal spacer sequence lengths. Accordingly, the spacer sequence may have 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the spacer sequence may have 18-24 nucleotides in length. In some embodiments, the targeting sequence may have 19-21 nucleotides in length. In some embodiments, the spacer sequence may comprise 20 nucleotides in length.

In some embodiments, the gRNA can be an sgRNA, which may comprise a 20 nucleotide spacer sequence at the 5′ end of the sgRNA sequence. In some embodiments, the sgRNA may comprise a less than 20 nucleotide spacer sequence at the 5′ end of the sgRNA sequence. In some embodiments, the sgRNA may comprise a more than 20 nucleotide spacer sequence at the 5′ end of the sgRNA sequence. In some embodiments, the sgRNA comprises a variable length spacer sequence with 17-30 nucleotides at the 5′ end of the sgRNA sequence. Examples are provided in Table 1 below. In these exemplary sequences, the fragment of “n” refers to the spacer sequence at the 5′ end.

In some embodiments, the sgRNA comprises comprise no uracil at the 3′ end of the sgRNA sequence. In other embodiments, the sgRNA may comprise one or more uracil at the 3′ end of the sgRNA sequence. For example, the sgRNA can comprise 1-8 uracil residues, at the 3′ end of the sgRNA sequence, e.g., 1, 2, 3, 4, 5, 6, 7, or 8 uracil residues at the 3′ end of the sgRNA sequence.

Any of the gRNAs disclosed herein, including any of the sgRNAs, may be unmodified. Alternatively, it may contain one or more modified nucleotides and/or modified backbones. For example, a modified gRNA such as an sgRNA can comprise one or more 2′-O-methyl phosphorothioate nucleotides, which may be located at either the 5′ end, the 3′ end, or both. In certain embodiments, more than one guide RNAs can be used with a CRISPR/Cas nuclease system. Each guide RNA may contain a different targeting sequence, such that the CRISPR/Cas system cleaves more than one target nucleic acid. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA is the same or different.

In some embodiments, the gRNAs disclosed herein target a TRAC gene. See also WO2019097305, the relevant disclosures of which are incorporated by reference herein for the subject matter and purpose referenced herein. Other gRNA sequences may be designed using the TRAC gene sequence located on chromosome 14 (GRCh38: chromosome 14: 22,547,506-22,552,154;. Ensembl; ENSG00000277734). In some embodiments, gRNAs targeting the TRAC genomic region and RNA-guided nuclease create breaks in the TRAC genomic region resulting Indels in the TRAC gene disrupting expression of the mRNA or protein. Exemplary spacer sequences and gRNAs targeting a TRAC gene are provided in Table 1 below.

In some embodiments, the gRNAs disclosed herein target a β2M gene, for example, target a suitable site within a β2M gene. See also WO2019097305, the relevant disclosures of which are incorporated by reference herein for the purpose and subject matter referenced herein. Other gRNA sequences may be designed using the β2M gene sequence located on Chromosome 15 (GRCh38 coordinates: Chromosome 15: 44,711,477-44,718,877; Ensembl: ENSG00000166710). In some embodiments, gRNAs targeting the β2M genomic region and RNA-guided nuclease create breaks in the β2M genomic region resulting in Indels in the β2M gene disrupting expression of the mRNA or protein. Exemplary spacer sequences and gRNAs targeting a β2M gene are provided in Table 1 below.

In some embodiments, the gRNAs disclosed herein target a CD70 gene, for example, target a site within exon 1 or exon 3 of a CD70 gene. See also WO2019/215500, the relevant disclosures of which are incorporated by reference herein for the purpose and subject matter referenced herein. Such a gRNA may comprise a spacer sequence complementary (complete or partially) to the target sequences in exon 1 or exon 3 of a CD70 gene, or a fragment thereof. Exemplary target sequences in a CD70 gene and exemplary gRNAs specific to the CD70 gene are provided in Table 1 below.

In some embodiments, the gRNAs disclosed herein target a TGFBRII gene, for example, target a site within exon 1, exon 2, exon 3, exon 4, exon 5, or exon 6 of the TGFBRII gene. Such a gRNA may comprise a spacer sequence complementary (complete or partially) to the target sequences in exon 2 or exon 4 of a TGFBRII gene, or a fragment thereof. Exemplary target sequences of TGFBRII and exemplary gRNA sequences are provided in Table 1 below:

In some embodiments, the gRNAs disclosed herein target a Reg1 gene, for example, target a site within exon 1, exon 2, exon 3, exon 4, exon 5, or exon 6 of the Reg1 gene. Such a gRNA may comprise a spacer sequence complementary (complete or partially) to the target sequences in exon 2 or exon 4 of a Reg1 gene, or a fragment thereof. Exemplary target sequences of Reg1 and exemplary gRNA sequences are provided in Table 1 below.

In some embodiments, the gRNAs disclosed herein target a TET2 gene, for example, target a site within exon 1, exon 3, exon 4, exon 5, exon 6, or a combination thereof within the TET2 gene. Such a gRNA may comprise a spacer sequence complementary (complete or partially) to the target sequences of a Reg1 gene, or a fragment thereof. Exemplary target sequences of TET2 and exemplary gRNA sequences are provided in Table 1 below. Additional information regarding TET2 knock-out can be found in International Application No. PCT/IB2020/058280, filed on Sep. 4, 2020, the relevant disclosures of which are incorporated by reference for the subject matter and purpose referenced herein.

By way of illustration, guide RNAs used in the CRISPR/Cas/Cpf1 system, or other smaller RNAs can be readily synthesized by chemical means, as illustrated below and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 or Cpf1 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

In some examples, the gRNAs of the present disclosure can be produced by in vitro transcription (IVT), synthetic and/or chemical synthesis methods, or a combination thereof. Enzymatic (IVT), solid-phase, liquid-phase, combined synthetic methods, small region synthesis, and ligation methods are utilized. In one embodiment, the gRNAs are made using IVT enzymatic synthesis methods. Methods of making polynucleotides by IVT are known in the art and are described in WO2013/151666. Accordingly, the present disclosure also includes polynucleotides, e.g., DNA, constructs and vectors are used to in vitro transcribe a gRNA described herein.

Various types of RNA modifications can be introduced during or after chemical synthesis and/or enzymatic generation of RNAs, e.g., modifications that enhance stability, reduce the likelihood or degree of innate immune response, and/or enhance other attributes, as described in the art. In some embodiments, non-natural modified nucleobases can be introduced into any of the gRNAs disclosed herein during synthesis or post-synthesis. In certain embodiments, modifications are on internucleoside linkages, purine or pyrimidine bases, or sugar. In some embodiments, a modification is introduced at the terminal of a gRNA with chemical synthesis or with a polymerase enzyme. Examples of modified nucleic acids and their synthesis are disclosed in WO2013/052523. Synthesis of modified polynucleotides is also described in Verma and Eckstein, Annual Review of Biochemistry, vol. 76, 99-134 (1998).

In some embodiments, enzymatic or chemical ligation methods can be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

In some embodiments of the present disclosure, a CRISPR/Cas nuclease system for use in genetically editing any of the target genes disclosed here may include at least one guide RNA. In some examples, the CRISPR/Cas nuclease system may contain multiple gRNAs, for example, 2, 3, or 4 gRNAs. Such multiple gRNAs may target different sites in a same target gene. Alternatively, the multiple gRNAs may target different genes. In some embodiments, the guide RNA(s) and the Cas protein may form a ribonucleoprotein (RNP), e.g., a CRISPR/Cas complex. The guide RNA(s) may guide the Cas protein to a target sequence(s) on one or more target genes as those disclosed herein, where the Cas protein cleaves the target gene at the target site. In some embodiments, the CRISPR/Cas complex is a Cpf1/guide RNA complex. In some embodiments, the CRISPR complex is a Type-II CRISPR/Cas9 complex. In some embodiments, the Cas protein is a Cas9 protein. In some embodiments, the CRISPR/Cas9 complex is a Cas9/guide RNA complex.

In some embodiments, the indel frequency (editing frequency) of a particular CRISPR/Cas nuclease system, comprising one or more specific gRNAs, may be determined using a TIDE analysis, which can be used to identify highly efficient gRNA molecules for editing a target gene. In some embodiments, a highly efficient gRNA yields a gene editing frequency of higher than 80%. For example, a gRNA is considered to be highly efficient if it yields a gene editing frequency of at least 80%, at least 85%, at least 90%, at least 95%, or 100%.

Delivery of Guide RNAs and Nucleases to T Cells

The CRISPR/Cas nuclease system disclosed herein, comprising one or more gRNAs and at least one RNA-guided nuclease, optionally a donor template as disclosed below, can be delivered to a target cell (e.g., a T cell) for genetic editing of a target gene, via a conventional method. In some embodiments, components of a CRISPR/Cas nuclease system as disclosed herein may be delivered to a target cell separately, either simultaneously or sequentially. In other embodiments, the components of the CRISPR/Cas nuclease system may be delivered into a target together, for example, as a complex. In some instances, gRNA and an RNA-guided nuclease can be pre-complexed together to form a ribonucleoprotein (RNP), which can be delivered into a target cell.

RNPs are useful for gene editing, at least because they minimize the risk of promiscuous interactions in a nucleic acid-rich cellular environment and protect the RNA from degradation. Methods for forming RNPs are known in the art. In some embodiments, an RNP containing an RNA-guided nuclease (e.g., a Cas nuclease, such as a Cas9 nuclease) and one or more gRNAs targeting one or more genes of interest can be delivered a cell (e.g., a T cell). In some embodiments, an RNP can be delivered to a T cell by electroporation.

In some embodiments, an RNA-guided nuclease can be delivered to a cell in a DNA vector that expresses the RNA-guided nuclease in the cell. In other examples, an RNA-guided nuclease can be delivered to a cell in an RNA that encodes the RNA-guided nuclease and expresses the nuclease in the cell. Alternatively or in addition, a gRNA targeting a gene can be delivered to a cell as a RNA, or a DNA vector that expresses the gRNA in the cell.

Delivery of an RNA-guided nuclease, gRNA, and/or an RNP may be through direct injection or cell transfection using known methods, for example, electroporation or chemical transfection. Other cell transfection methods may be used.

Other Gene Editing Methods

Besides the CRISPR method disclosed herein, additional gene editing methods as known in the art can also be used in making the genetically engineered T cells disclosed herein. Some examples include gene editing approaching involve zinc finger nuclease (ZFN), transcription activator-like effector nucleases (TALEN), restriction endonucleases, meganucleases homing endonucleases, and the like.

ZFNs are targeted nucleases comprising a nuclease fused to a zinc finger DNA binding domain (ZFBD), which is a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A selected zinc finger domain is a domain not found in nature whose production results primarily from an empirical process such as phage display, interaction trap or hybrid selection. ZFNs are described in greater detail in U.S. Pat. Nos. 7,888,121 and 7,972,854. The most recognized example of a ZFN is a fusion of the FokI nuclease with a zinc finger DNA binding domain.

A TALEN is a targeted nuclease comprising a nuclease fused to a TAL effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Patent Application No. 2011/0145940. The most recognized example of a TALEN in the art is a fusion polypeptide of the FokI nuclease to a TAL effector DNA binding domain.

Additional examples of targeted nucleases suitable for use as provided herein include, but are not limited to, Bxb1, phiC31, R4, PhiBT1, and Wβ/SPBc/TP901-1, whether used individually or in combination.

Any of the nucleases disclosed herein may be delivered using a vector system, including, but not limited to, plasmid vectors, DNA minicircles, retroviral vectors, lentiviral vectors, adenovirus vectors, poxvirus vectors; herpesvirus vectors and adeno-associated virus vectors, and combinations thereof.

Conventional viral and non-viral based gene transfer methods can be used to introduce nucleic acids encoding nucleases and donor templates in cells (e.g., T cells). Non-viral vector delivery systems include DNA plasmids, DNA minicircles, naked nucleic acid, and nucleic acid complexed with a delivery vehicle such as a liposome or poloxamer. Viral vector delivery systems include DNA and RNA viruses, which have either episomal or integrated genomes after delivery to the cell.

Methods of non-viral delivery of nucleic acids include electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids. Some specific examples are provided below.

C. Chimeric Antigen Receptor (CAR)

In some embodiments, the genetically engineered T cells disclosed herein are CAR-T cells, which express a chimeric antigen receptor (CAR). A chimeric antigen receptor (CAR) refers to an artificial immune cell receptor that is engineered to recognize and bind to an antigen expressed by undesired cells, for example, disease cells such as cancer cells. A T cell that expresses a CAR polypeptide is referred to as a CAR T cell. CARs have the ability to redirect T-cell specificity and reactivity toward a selected target in a non-MHC-restricted manner. The non-MHC-restricted antigen recognition gives CAR-T cells the ability to recognize an antigen independent of antigen processing, thus bypassing a major mechanism of tumor escape. Moreover, when expressed on T-cells, CARs advantageously do not dimerize with endogenous T-cell receptor (TCR) alpha and beta chains.

There are various generations of CARs, each of which contains different components. First generation CARs join an antibody-derived scFv to the CD3zeta or z) intracellular signaling domain of the T-cell receptor through hinge and transmembrane domains. Second generation CARs incorporate an additional co-stimulatory domain, e.g., CD28, 4-1BB (41BB), or ICOS, to supply a costimulatory signal. Third-generation CARs contain two costimulatory domains (e.g., a combination of CD27, CD28, 4-1BB, ICOS, or OX40) fused with the TCR CD3ζ chain. Maude et al., Blood. 2015; 125(26):4017-4023; Kakarla and Gottschalk, Cancer J. 2014; 20(2):151-155). Any of the various generations of CAR constructs is within the scope of the present disclosure.

Generally, a CAR is a fusion polypeptide comprising an extracellular domain that recognizes a target antigen (e.g., a single chain fragment (scFv) of an antibody or other antibody fragment) and an intracellular domain comprising a signaling domain of the T-cell receptor (TCR) complex (e.g., CD3ζ) and, in most cases, a co-stimulatory domain. (Enblad et al., Human Gene Therapy. 2015; 26(8):498-505). A CAR construct may further comprise a hinge and transmembrane domain between the extracellular domain and the intracellular domain, as well as a signal peptide at the N-terminus for surface expression. Examples of signal peptides include SEQ ID NO: 80 and SEQ ID NO: 81 as provided in Table 7 below. Other signal peptides may be used.

(i) Antigen Binding Extracellular Domain

The antigen-binding extracellular domain is the region of a CAR polypeptide that is exposed to the extracellular fluid when the CAR is expressed on cell surface. In some instances, a signal peptide may be located at the N-terminus to facilitate cell surface expression. In some embodiments, the antigen binding domain can be a single-chain variable fragment (scFv, which may include an antibody heavy chain variable region (V_{H}) and an antibody light chain variable region (V_{L}) (in either orientation). In some instances, the V_{H} and V_{L} fragment may be linked via a peptide linker. The linker, in some embodiments, includes hydrophilic residues with stretches of glycine and serine for flexibility as well as stretches of glutamate and lysine for added solubility. The scFv fragment retains the antigen-binding specificity of the parent antibody, from which the scFv fragment is derived. In some embodiments, the scFv may comprise humanized V_{H} and/or V_{L} domains. In other embodiments, the V_{H} and/or V_{L} domains of the scFv are fully human.

The antigen-binding extracellular domain may be specific to a target antigen of interest, for example, a pathologic antigen such as a tumor antigen (e.g., a solid tumor antigen). In some embodiments, a tumor antigen is a “tumor associated antigen,” referring to an immunogenic molecule, such as a protein, that is generally expressed at a higher level in tumor cells than in non-tumor cells, in which it may not be expressed at all, or only at low levels. In some embodiments, tumor-associated structures, which are recognized by the immune system of the tumor-harboring host, are referred to as tumor-associated antigens. In some embodiments, a tumor-associated antigen is a universal tumor antigen, if it is broadly expressed by most types of tumors. In some embodiments, tumor-associated antigens are differentiation antigens, mutational antigens, overexpressed cellular antigens or viral antigens. In some embodiments, a tumor antigen is a “tumor specific antigen” or “TSA,” referring to an immunogenic molecule, such as a protein, that is unique to a tumor cell. Tumor specific antigens are exclusively expressed in tumor cells, for example, in a specific type of tumor cells.

Exemplary tumor antigens include, but are not limited to, CD19, BCMA, and CD70. Any known antibodies specific to such tumor antigens, for example, those approved for marketing and those in clinical trials, can be used for making the CAR constructs disclosed herein. Non-limiting examples of CAR constructs are provided in WO2019097305 and WO2019215500, and WO2020/095107, the relevant disclosures of which are herein incorporated by reference for the purposes and subject matter referenced herein.

(ii) Transmembrane Domain

The CAR polypeptide disclosed herein may contain a transmembrane domain, which can be a hydrophobic alpha helix that spans the membrane. As used herein, a “transmembrane domain” refers to any protein structure that is thermodynamically stable in a cell membrane, preferably a eukaryotic cell membrane. The transmembrane domain can provide stability of the CAR containing such.

In some embodiments, the transmembrane domain of a CAR as provided herein can be a CD8 transmembrane domain. In other embodiments, the transmembrane domain can be a CD28 transmembrane domain. In yet other embodiments, the transmembrane domain is a chimera of a CD8 and CD28 transmembrane domain Other transmembrane domains may be used as provided herein. In some embodiments, the transmembrane domain is a CD8a transmembrane domain containing the sequence of SEQ ID NO: 82 as provided below in Table 7. Other transmembrane domains may be used.

(iii) Hinge Domain

In some embodiments, a hinge domain may be located between an extracellular domain (comprising the antigen binding domain) and a transmembrane domain of a CAR, or between a cytoplasmic domain and a transmembrane domain of the CAR. A hinge domain can be any oligopeptide or polypeptide that functions to link the transmembrane domain to the extracellular domain and/or the cytoplasmic domain in the polypeptide chain. A hinge domain may function to provide flexibility to the CAR, or domains thereof, or to prevent steric hindrance of the CAR, or domains thereof.

In some embodiments, a hinge domain may comprise up to 300 amino acids (e.g., 10 to 100 amino acids, or 5 to 20 amino acids). In some embodiments, one or more hinge domain(s) may be included in other regions of a CAR. In some embodiments, the hinge domain may be a CD8 hinge domain. Other hinge domains may be used.

(iv) Intracellular Signaling Domains

Any of the CAR constructs contain one or more intracellular signaling domains (e.g., CD3ζ, and optionally one or more co-stimulatory domains), which are the functional end of the receptor. Following antigen recognition, receptors cluster and a signal is transmitted to the cell.

CD3ζ is the cytoplasmic signaling domain of the T cell receptor complex. CD3ζ contains three (3) immunoreceptor tyrosine-based activation motif (ITAM)s, which transmit an activation signal to the T cell after the T cell is engaged with a cognate antigen. In many cases, CD3ζ provides a primary T cell activation signal but not a fully competent activation signal, which requires a co-stimulatory signaling.

In some embodiments, the CAR polypeptides disclosed herein may further comprise one or more co-stimulatory signaling domains. For example, the co-stimulatory domains of CD28 and/or 4-1BB may be used to transmit a full proliferative/survival signal, together with the primary signaling mediated by CD3ζ. In some examples, the CAR disclosed herein comprises a CD28 co-stimulatory molecule. In other examples, the CAR disclosed herein comprises a 4-1BB co-stimulatory molecule. In some embodiments, a CAR includes a CD3ζ signaling domain and a CD28 co-stimulatory domain. In other embodiments, a CAR includes a CD3ζ signaling domain and 4-1BB co-stimulatory domain. In still other embodiments, a CAR includes a CD3ζ signaling domain, a CD28 co-stimulatory domain, and a 4-1BB co-stimulatory domain.

Table 7 provides examples of signaling domains derived from 4-1BB, CD28 and CD3-zeta that may be used herein.

In specific examples, the anti-CD19 CAR disclosed herein may comprise the amino acid sequence of SEQ ID NO: 101, which may be encoded by the nucleotide sequence of SEQ ID NO: 100. In other examples, the anti-BCMA CAR disclosed herein may comprise the amino acid sequence of SEQ ID NO: 131, which may be encoded by the nucleotide sequence of SEQ ID NO: 130. In other examples, the anti-CD70 CAR disclosed herein may comprise the amino acid sequence of SEQ ID NO: 123, which may be encoded by the nucleotide sequence of SEQ ID NO: 122. See sequences of various CAR constructs and components thereof in Table 7 below, all of which are within the scope of the present disclosure.

(b) Delivery of CAR Construct to T Cells

In some embodiments, a nucleic acid encoding a CAR can be introduced into any of the genetically engineered T cells disclosed herein by methods known to those of skill in the art. For example, a coding sequence of the CAR may be cloned into a vector, which may be introduced into the genetically engineered T cells for expression of the CAR. A variety of different methods known in the art can be used to introduce any of the nucleic acids or expression vectors disclosed herein into an immune effector cell. Non-limiting examples of methods for introducing nucleic acid into a cell include: lipofection, transfection (e.g., calcium phosphate transfection, transfection using highly branched organic compounds, transfection using cationic polymers, dendrimer-based transfection, optical transfection, particle-based transfection (e.g., nanoparticle transfection), or transfection using liposomes (e.g., cationic liposomes)), microinjection, electroporation, cell squeezing, sonoporation, protoplast fusion, impalefection, hydrodynamic delivery, gene gun, magnetofection, viral transfection, and nucleofection.

In specific examples, a nucleic acid encoding a CAR construct can be delivered to a cell using an adeno-associated virus (AAV). AAVs are small viruses which integrate site-specifically into the host genome and can therefore deliver a transgene, such as CAR. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids which confer AAV serotype, which determines which target organs the capsids will primarily bind and thus what cells the AAV will most efficiently infect. There are twelve currently known human AAV serotypes. In some embodiments, the AAV for use in delivering the CAR-coding nucleic acid is AAV serotype 6 (AAV6).

Adeno-associated viruses are among the most frequently used viruses for gene therapy for several reasons. First, AAVs do not provoke an immune response upon administration to mammals, including humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

A nucleic acid encoding a CAR can be designed to insert into a genomic site of interest in the host T cells. In some embodiments, the target genomic site can be in a safe harbor locus.

In some embodiments, a nucleic acid encoding a CAR (e.g., via a donor template, which can be carried by a viral vector such as an adeno-associated viral (AAV) vector) can be designed such that it can insert into a location within a TRAC gene to disrupt the TRAC gene in the genetically engineered T cells and express the CAR polypeptide. Disruption of TRAC leads to loss of function of the endogenous TCR. For example, a disruption in the TRAC gene can be created with an endonuclease such as those described herein and one or more gRNAs targeting one or more TRAC genomic regions. Any of the gRNAs specific to a TRAC gene and the target regions disclosed herein can be used for this purpose.

In some examples, a genomic deletion in the TRAC gene and replacement by a CAR coding segment can be created by homology directed repair or HDR (e.g., using a donor template, which may be part of a viral vector such as an adeno-associated viral (AAV) vector). In some embodiments, a disruption in the TRAC gene can be created with an endonuclease as those disclosed herein and one or more gRNAs targeting one or more TRAC genomic regions and inserting a CAR coding segment into the TRAC gene.

A donor template as disclosed herein can contain a coding sequence for a CAR. In some examples, the CAR-coding sequence may be flanked by two regions of homology to allow for efficient HDR at a genomic location of interest, for example, at a TRAC gene using a gene editing method known in the art. In some examples, a CRISPR-based method can be used. In this case, both strands of the DNA at the target locus can be cut by a CRISPR Cas9 enzyme guided by gRNAs specific to the target locus. HDR then occurs to repair the double-strand break (DSB) and insert the donor DNA coding for the CAR. For this to occur correctly, the donor sequence is designed with flanking residues which are complementary to the sequence surrounding the DSB site in the target gene (hereinafter “homology arms”), such as the TRAC gene. These homology arms serve as the template for DSB repair and allow HDR to be an essentially error-free mechanism. The rate of homology directed repair (HDR) is a function of the distance between the mutation and the cut site so choosing overlapping or nearby target sites is important. Templates can include extra sequences flanked by the homologous regions or can contain a sequence that differs from the genomic sequence, thus allowing sequence editing.

Alternatively, a donor template may have no regions of homology to the targeted location in the DNA and may be integrated by NHEJ-dependent end joining following cleavage at the target site.

A donor template can be DNA or RNA, single-stranded and/or double-stranded, and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al., (1987) Proc. Natl. Acad. Sci. USA 84:4959-4963; Nehls et al., (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor template can be introduced into a cell as part of a vector molecule having additional sequences such as, for example, replication origins, promoters and genes encoding antibiotic resistance. Moreover, a donor template can be introduced into a cell as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLY)).

A donor template, in some embodiments, can be inserted at a site nearby an endogenous prompter (e.g., downstream or upstream) so that its expression can be driven by the endogenous promoter. In other embodiments, the donor template may comprise an exogenous promoter and/or enhancer, for example, a constitutive promoter, an inducible promoter, or tissue-specific promoter to control the expression of the CAR gene. In some embodiments, the exogenous promoter is an EF1α promoter, see, e.g., SEQ ID NO: 152 provided in Table 8 below. Other promoters may be used.

Furthermore, exogenous sequences may also include transcriptional or translational regulatory sequences, for example, promoters, enhancers, insulators, internal ribosome entry sites, sequences encoding 2A peptides and/or polyadenylation signals.

When needed, additional gene editing (e.g., gene knock-in or knock-out) can be introduced into therapeutic T cells as disclosed herein to improve T cell function and therapeutic efficacy. For example, if β2M knockout can be performed to reduce the risk of or prevent a host-versus-graft response. Other examples include knock-in or knock-out genes to improve target cell lysis, knock-in or knock-out genes to enhance performance of therapeutic T cells such as CAR-T cells.

In some embodiments, a donor template for delivering an anti-CD19 CAR may be an AAV vector inserted with a nucleic acid fragment comprising the coding sequence of the anti-CD19 CAR, and optionally regulatory sequences for expression of the anti-CD19 CAR (e.g., a promoter such as the EF1a promoter provided in the sequence Table), which can be flanked by homologous arms for inserting the coding sequence and the regulatory sequences into a genomic locus of interest. In some examples, the nucleic acid fragment is inserted in the endogenous TRAC gene locus, thereby disrupting expression of the TRAC gene. In specific examples, the nucleic acid may replace a fragment in the TRAC gene, for example, a fragment comprising the nucleotide sequence of AGAGCAACAGTGCTGTGGCC (SEQ ID NO: 29). In some specific examples, the donor template for delivering the anti-CD19 CAR may comprise a nucleotide sequence of SEQ ID NO: 153, which can be inserted into a disrupted TRAC gene, for example, replacing the fragment of SEQ ID NO: 29. See Table 8 below.

In some embodiments, a donor template for delivering an anti-BCMA CAR may be an AAV vector inserted with a nucleic acid fragment comprising the coding sequence of the anti-BCMA CAR, and optionally regulatory sequences for expression of the anti-BCMA CAR (e.g., a promoter such as the EF1a promoter provided in the sequence Table), which can be flanked by homologous arms for inserting the coding sequence and the regulatory sequences into a genomic locus of interest. In some examples, the nucleic acid fragment is inserted in the endogenous TRAC gene locus, thereby disrupting expression of the TRAC gene. In specific examples, the nucleic acid may replace a fragment in the TRAC gene, for example, a fragment comprising the nucleotide sequence of SEQ ID NO: 29. In some specific examples, the donor template for delivering the anti-BCMA CAR may comprise a nucleotide sequence of SEQ ID NO: 155, which can be inserted into a disrupted TRAC gene, for example, replacing the fragment of SEQ ID NO: 29. See Table 8 below.

In some embodiments, a donor template for delivering an anti-CD70 CAR may be an AAV vector inserted with a nucleic acid fragment comprising the coding sequence of the anti-CD70 CAR, and optionally regulatory sequences for expression of the anti-CD70 CAR (e.g., a promoter such as the EF1a promoter provided in the sequence Table), which can be flanked by homologous arms for inserting the coding sequence and the regulatory sequences into a genomic locus of interest. In some examples, the nucleic acid fragment is inserted in the endogenous TRAC gene locus, thereby disrupting expression of the TRAC gene. In specific examples, the nucleic acid may replace a fragment in the TRAC gene, for example, a fragment comprising the nucleotide sequence of SEQ ID NO: 29. In some specific examples, the donor template for delivering the anti-CD70 CAR may comprise a nucleotide sequence of SEQ ID NO: 154, which can be inserted into a disrupted TRAC gene, for example, replacing the fragment of SEQ ID NO: 29. See Table 8 below.

The genetically engineered T cells having a disrupted TGFBRII gene, one or more additional disrupted genes, e.g., β2M, TRAC, CD70, and/or Reg1, and further expressing a chimeric antigen receptor (CAR) can be produced by sequential targeting of the genes of interest. For example, in some embodiments, the TGFBRII gene may be disrupted first, followed by disruption of TRAC, β2M, and/or Reg1 genes and CAR insertion. In other embodiments, TRAC and β2M genes may be disrupted first, followed by CAR insertion and disruption of the TGFBRII gene, and optionally the Reg1 gene. In other embodiments, CD70 may be disrupted first, followed by TRAC and β2M genes disruption and CAR insertion, as well as disruptions of the TGFBRII gene and optionally the Reg1 gene. Accordingly, in some embodiments, the genetically engineered T cells disclosed herein may be produced by multiple, sequential electroporation events with multiple RNPs targeting the genes of interest, e.g., β2M, TRAC, CD70, TGFBRII, Reg1, TET2, etc.

In other embodiments, the genetically engineered CAR T cells disclosed herein may be produced by a single electroporation event with an RNP complex comprising an RNA-guided nuclease and multiple gRNAs targeting the genes of interest, e.g., β2M, TRAC, CD70, TGFBRII, Reg1, TET2, etc.

(c) Exemplary Genetically Engineered T Cells Expressing a Chimeric Antigen Receptor

It should be understood that gene disruption encompasses gene modification through gene editing (e.g., using CRISPR/Cas gene editing to insert or delete one or more nucleotides). A disrupted gene may contain one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or expresses a substantially reduced level of the encoded protein. In other instances, the disrupted gene expresses the encoded protein in a mutated form, which is either not functional or has substantially reduced activity. In some embodiments, a disrupted gene is a gene that does not encode functional protein. In some embodiments, a cell that comprises a disrupted gene does not express (e.g., at the cell surface) a detectable level (e.g. by antibody, e.g., by flow cytometry) of the protein encoded by the gene. A cell that does not express a detectable level of the protein may be referred to as a knockout cell. For example, a cell having a β2M gene edit may be considered a β2M knockout cell if β2M protein cannot be detected at the cell surface using an antibody that specifically binds β2M protein.

i. Anti-CD19 CAR T Cells

Also provided herein is population of genetically engineered immune cells (e.g., T cells such as human T cells) expressing an anti-CD19 CAR, e.g., those disclosed herein. In some examples, the anti-CD19 CAR-T cells disclosed herein, which express any of the anti-CD19 CAR disclosed herein (e.g., the anti-CD19 CAR comprising the amino acid sequence of SEQ ID NO: 102), may also comprise a disrupted TRAC gene and/or a disrupted β2M gene as also disclosed herein.

In some examples, anti-CD19 CAR cells are CD19-directed T cells having disrupted TRAC gene and β2M gene. The nucleic acid encoding the anti-CD19 CAR can be inserted in the disrupted TRAC gene at the site of SEQ ID NO: 29, which is replaced by the nucleic acid encoding the anti-CD19 CAR, thereby disrupting expression of the TRAC gene. The disrupted TRAC gene in the anti-CD19 CAR cells may comprise the nucleotide sequence of SEQ ID NO: 153.

The anti-CD19 CAR-T cells disclosed herein may further comprise one or more edited genes, for example, a disrupted CD70 gene, a disrupted TGFBRII gene, a disrupted Reg1 gene, a disrupted TET2 gene, or a combination thereof.

Anti-CD19 CAR T cells can be produced via ex vivo genetic modification using the CRISPR/Cas9 (Clustered Regularly Interspaced Short Palindromic Repeats/CRISPR associated protein 9) technology to disrupt one or more targeted genes (e.g., those disclosed herein), and adeno-associated virus (AAV) transduction to deliver the anti-CD19 CAR construct. CRISPR-Cas9-mediated gene editing involves one or more guide RNAs (sgRNAs), for example, a sgRNA that targets the TRAC locus (e.g., TA-1, see Table 1), a sgRNA that target the β2M locus (e.g., β2M−1; see Table 1), and one or more sgRNAs targeting one or more additional target genes (see also Table 1). For any of the gRNA sequences provided herein, those that do not explicitly indicate modifications are meant to encompass both unmodified sequences and sequences having any suitable modifications.

The anti-CD19 CAR T cells are composed of an anti-CD19 single-chain antibody fragment (scFv, which may comprise the amino acid sequence of SEQ ID NO: 104), followed by a CD8 hinge and transmembrane domain (e.g., comprising the amino acid sequence of SEQ ID NO: 107) that is fused to an intracellular co-signaling domain of CD28 (e.g., SEQ ID NO: 86) and a CD3ζ signaling domain (e.g., SEQ ID NO: 88). In specific examples, the anti-CD19 CAR T cells comprises the amino acid sequence of SEQ ID NO: 102. See Table 7.

In some embodiments, at least 30% of a population of anti-CD19 CAR T cells express a detectable level of the anti-CD19 CAR. For example, at least 40%, at least 50%, at least 60%, at least 70%, at least 75%, at least 80%, at least 85%, at least 90%, or at least 95% of the anti-CD19 CAR T cells express a detectable level of the anti-CD19 CAR.

In some embodiments, at least 50% of a population of anti-CD19 CAR T cells may not express a detectable level of β2M surface protein. For example, at least 55%, at least 60%, at least 70%, at least 75%, at least 80%, at least 85%, at least 90%, or at least 95% of the anti-CD19 CAR T cells may not express a detectable level of β2M surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of β2M surface protein.

Alternatively or in addition, at least 50% of a population of anti-CD19 CAR T cells may not express a detectable level of TRAC surface protein. For example, at least 55%, at least 60%, at least 70%, at least 75%, at least 80%, at least 85%, at least 90%, or at least 95% of the anti-CD19 CAR T cells may not express a detectable level of TRAC surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of TRAC surface protein. In specific examples, more than 90% (e.g., more than 99.5%) of the anti-CD19 CAR T cells do not express a detectable TRAC surface protein.

In some embodiments, a substantial percentage of the population of anti-CD19 CAR T cells may comprise more than one gene edit, which results in a certain percentage of cells not expressing more than one gene and/or protein.

For example, at least 50% of a population of anti-CD19 CAR T cells may not express a detectable level of two surface proteins, e.g., does not express a detectable level of β2M and TRAC proteins. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the anti-CD19 CAR T cells do not express a detectable level of TRAC and β2M surface proteins. In another example, at least 50% of a population of the anti-CD19 CAR T cells do not express a detectable level of TRAC and β2M surface proteins.

In some embodiments, the population of anti-CD19 CAR T cells may comprise more than one gene edit (e.g., in more than one gene), which may be an edit described herein. For example, the population of anti-CD19 CAR T cells may comprise a disrupted TRAC gene via the CRISPR/Cas technology using the TA-1 TRAC gRNA. In some examples, the anti-CD19 CART cells may comprise a deletion in the TRAC gene relative to unmodified T cells. For example, the anti-CD19 CAR T cells may comprise a deletion of the fragment AGAGCAACAGTGCTGTGGCC (SEQ ID NO: 29) in the TRAC gene. This fragment can be replaced by the nucleic acid encoding the anti-CD19 CAR (e.g., SEQ ID NO: 153).

Alternatively or in addition, the population of anti-CD19 CAR T cells may comprise a disrupted β2M gene via CRISPR/Cas9 technology using the gRNA of β2M−1. Such anti-CD19 CAR T cells may comprise Indels in the β2M gene, which comprise one or more of the nucleotide sequences of SEQ ID NOs: 57-62. See Table 3. In specific examples, anti-CD19 CAR T cells comprise ≥30% CARP T cells, ≤50% β2M+ cells, and ≤30% TCRαβ⁺ cells. In additional specific examples, anti-CD19 CAR T cells comprise ≥30% CARP T cells, ≤30% β2M+ cells, and ≤0.5% TCRαβ+ cells. See also WO 2019/097305A2, and WO2019215500, the relevant disclosures of each of which are incorporated by reference for the subject matter and purpose referenced herein.

ii Anti-BCMA CAR-T Cells

Also provided herein is population of genetically engineered immune cells (e.g., T cells such as human T cells) expressing an anti-BCMA CAR, e.g., those disclosed herein. In some examples, the anti-BCMA CAR T cells disclosed herein, which express any of the anti-BCMA CAR disclosed herein (e.g., the anti-BCMA CAR comprising the amino acid sequence of SEQ ID NO: 131), may also comprise a disrupted TRAC gene and/or a disrupted β2M gene as also disclosed herein.

In some examples, the anti-BCMA CAR T cells comprise disrupted TRAC gene and β2M gene. The nucleic acid encoding the anti-BCMA CAR can be inserted in the disrupted TRAC gene at the site of SEQ ID NO: 29, which is replaced by the nucleic acid encoding the anti-BCMA CAR, thereby disrupting expression of the TRAC gene. The disrupted TRAC gene in the anti-BCMA CAR T cells may comprise the nucleotide sequence of SEQ ID NO: 155.

The anti-BCMA CAR-T cells disclosed herein may further comprise one or more edited genes, for example, a disrupted CD70 gene, a disrupted TGFBRII gene, a disrupted Reg1 gene, a disrupted TET2 gene, or a combination thereof.

Anti-BCMA CAR T cells can be produced via ex vivo genetic modification using the CRISPR/Cas9 (Clustered Regularly Interspaced Short Palindromic Repeats/CRISPR associated protein 9) technology to disrupt one or more targeted genes (e.g., those disclosed herein), and adeno-associated virus (AAV) transduction to deliver the anti-BCMA CAR construct. CRISPR-Cas9-mediated gene editing involves one or more guide RNAs (sgRNAs), for example, a sgRNA that targets the TRAC locus (e.g., TA-1, see Table 1), a sgRNA that target the β2M locus (e.g., β2M−1; see Table 1), and one or more sgRNAs targeting one or more additional target genes (see also Table 1). For any of the gRNA sequences provided herein, those that do not explicitly indicate modifications are meant to encompass both unmodified sequences and sequences having any suitable modifications.

The anti-BCMA CAR T cells are composed of an anti-BCMA single-chain antibody fragment (scFv, which may comprise the amino acid sequence of SEQ ID NO: 133), followed by a CD8 hinge and transmembrane domain (e.g., comprising the amino acid sequence of SEQ ID NO: 107) that is fused to an intracellular co-signaling domain of 4-1BB (e.g., SEQ ID NO: 84) and a CD3ζ signaling domain (e.g., SEQ ID NO: 88). In specific examples, the anti-BCMA CAR T cells comprises the amino acid sequence of SEQ ID NO: 131.

In some embodiments, at least 30% of a population of anti-BCMA CAR T cells express a detectable level of the anti-BCMA CAR. For example, at least 40%, at least 50%, at least 60%, at least 70%, at least 75%, at least 80%, at least 85%, at least 90%, or at least 95% of the anti-BCMA CAR T cells express a detectable level of the anti-BCMA CAR.

In some embodiments, at least 50% of a population of anti-BCMA CAR T cells may not express a detectable level of β2M surface protein. For example, at least 55%, at least 60%, at least 70%, at least 75%, at least 80%, at least 85%, at least 90%, or at least 95% of the anti-BCMA CAR T cells may not express a detectable level of β2M surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of β2M surface protein.

Alternatively or in addition, at least 50% of a population of anti-BCMA CAR T cells may not express a detectable level of TRAC surface protein. For example, at least 55%, at least 60%, at least 70%, at least 75%, at least 80%, at least 85%, at least 90%, or at least 95% of the anti-BCMA CAR T cells may not express a detectable level of TRAC surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of TRAC surface protein. In specific examples, more than 90% (e.g., more than 99.5%) of the anti-BCMA CAR T cells do not express a detectable TRAC surface protein.

In some embodiments, a substantial percentage of the population of anti-BCMA CAR T cells may comprise more than one gene edit, which results in a certain percentage of cells not expressing more than one gene and/or protein.

For example, at least 50% of a population of anti-BCMA CAR T cells may not express a detectable level of two surface proteins, e.g., does not express a detectable level of β2M and TRAC proteins. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the anti-BCMA CAR T cells do not express a detectable level of TRAC and β2M surface proteins. In another example, at least 50% of a population of anti-BCMA CAR T cells do not express a detectable level of TRAC and β2M surface proteins.

In some embodiments, the population of anti-BCMA CAR T cells may comprise more than one gene edit (e.g., in more than one gene), which may be an edit described herein. For example, the population of anti-BCMA CAR T cells may comprise a disrupted TRAC gene via the CRISPR/Cas technology using the TA-1 TRAC gRNA. In some examples, the anti-BCMA CAR T cells may comprise a deletion in the TRAC gene relative to unmodified T cells. For example, the anti-BCMA CAR T cells may comprise a deletion of the fragment AGAGCAACAGTGCTGTGGCC (SEQ ID NO: 29) in the TRAC gene. This fragment can be replaced by the nucleic acid encoding the anti-BCMA CAR (e.g., SEQ ID NO: 155).

Alternatively or in addition, the population of anti-BCMA CAR T cells may comprise a disrupted β2M gene via CRISPR/Cas9 technology using the gRNA of β2M−1. Such anti-BCMA CAR T cells may comprise Indels in the β2M gene, which comprise one or more of the nucleotide sequences of SEQ ID NOs: 57-62. See Table 3. In specific examples, anti-BCMA CAR T cells comprise ≥30% CARP T cells, ≤50% β2M+ cells, and ≤30% TCRαβ+ cells. In additional specific examples, anti-BCMA CAR T cells comprise ≥30% CARP T cells, ≤30% β2M+ cells, and ≤0.5% TCRαβ+ cells. See also WO 2019/097305A2, and WO2019215500, the relevant disclosures of each of which are incorporated by reference for the subject matter and purpose referenced herein.

iii. Anti-CD70 CAR-T Cells

Also provided herein is population of genetically engineered immune cells (e.g., T cells such as human T cells) expressing anti-CD70 CAR, e.g., those disclosed herein. In some examples, the anti-CD70 CAR T cells disclosed herein, which express any of the anti-CD70 CAR disclosed herein (e.g., the anti-CD70 CAR comprising the amino acid sequence of SEQ ID NO: 123), may also comprise a disrupted TRAC gene, a disrupted β2M gene, and/or a disrupted CD70 gene as also disclosed herein.

In some examples anti-CD70 CAR T cells are anti-CD70 CAR T cells having disrupted TRAC gene, a disrupted β2M gene, and a disrupted CD70 gene. The nucleic acid encoding the anti-CD70 CAR can be inserted in the disrupted TRAC gene at the site of SEQ ID NO: 29, which is replaced by the nucleic acid encoding the anti-CD70 CAR, thereby disrupting expression of the TRAC gene. The disrupted TRAC gene in the anti-CD70 CAR T cells may comprise the nucleotide sequence of SEQ ID NO: 154.

The anti-CD70 CAR-T cells disclosed herein may further comprise one or more edited genes, for example, a disrupted TGFBRII gene, a disrupted Reg1 gene, a disrupted TET2 gene, or a combination thereof.

Anti-CD70 CAR T cells can be produced via ex vivo genetic modification using the CRISPR/Cas9 (Clustered Regularly Interspaced Short Palindromic Repeats/CRISPR associated protein 9) technology to disrupt targeted genes, and adeno-associated virus (AAV) transduction to deliver the anti-CD70 CAR construct. CRISPR-Cas9-mediated gene editing involves guide RNAs (sgRNAs): an sgRNA which targets the CD70 locus (e.g., CD70-7, see Table 1), a sgRNA that targets the TRAC locus (e.g., TA-1, see Table 1), and a sgRNA that target the β2M locus (e.g., β2M−1; see Table 1), as well as sgRNAs targeting one or more additional genes such as those disclosed herein.

The anti-CD70 CAR T cells are composed of an anti-CD70 CAR single-chain antibody fragment (scFv, which may comprise the amino acid sequence of SEQ ID NO: 125 or SEQ ID NO:127), followed by a CD8 hinge and transmembrane domain (e.g., comprising the amino acid sequence of SEQ ID NO: 107) that is fused to an intracellular co-signaling domain of 4-1BB (e.g., SEQ ID NO: 84) and a CD3ζ signaling domain (e.g., SEQ ID NO: 88). In specific examples, the anti-CD70 CAR T cells comprise the amino acid sequence of SEQ ID NO: 123.

In some embodiments, at least 30% of a population of anti-CD70 CAR T cells express a detectable level of the anti-CD70 CAR. For example, at least 40%, at least 50%, at least 60%, at least 70%, at least 75%, at least 80%, at least 85%, at least 90%, or at least 95% of the anti-CD70 CAR T cells express a detectable level of the anti-CD70 CAR.

In some embodiments, at least 50% of a population of anti-CD70 CAR T cells may not express a detectable level of β2M surface protein. For example, at least 55%, at least 60%, at least 70%, at least 75%, at least 80%, at least 85%, at least 90%, or at least 95% of the anti-CD70 CAR T cells may not express a detectable level of β2M surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of β2M surface protein.

Alternatively or in addition, at least 50% of a population of anti-CD70 CAR T cells may not express a detectable level of TRAC surface protein. For example, at least 55%, at least 60%, at least 70%, at least 75%, at least 80%, at least 85%, at least 90%, or at least 95% of the anti-CD70 CAR T cells may not express a detectable level of TRAC surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of TRAC surface protein. In specific examples, more than 90% (e.g., more than 99.5%) of the anti-CD70 CAR T cells do not express a detectable TRAC surface protein.

In some embodiments, at least 50% of a population of the anti-CD70 CAR T cells may not express a detectable level of CD70 surface protein. For example, at least 55%, at least 60%, at least 70%, at least 75%, at least 80%, at least 85%, at least 90%, at least 95%, or at least 98% of the engineered T cells of a population may not express a detectable level of CD70 surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, 90%-100%, or 95%-100% of the engineered T cells of a population does not express a detectable level of CD70 surface protein.

In some embodiments, a substantial percentage of the population of anti-CD70 CAR T cells may comprise more than one gene edit, which results in a certain percentage of cells not expressing more than one gene and/or protein.

For example, at least 50% of a population of anti-CD70 CAR T cells may not express a detectable level of two surface proteins, e.g., does not express a detectable level of β2M and TRAC proteins, β2M and CD70 proteins, or TRAC and CD70 proteins. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of two surface proteins. In another example, at least 50% of a population of the anti-CD70 CAR T cells may not express a detectable level of all of the three target surface proteins β2M, TRAC, and CD70 proteins. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of β2M, TRAC, and CD70 surface proteins.

In some embodiments, the population of anti-CD70 CAR T cells may comprise more than one gene edit (e.g., in more than one gene), which may be an edit described herein. For example, the population of anti-CD70 CAR T cells may comprise a disrupted TRAC gene via the CRISPR/Cas technology using the TA-1 TRAC gRNA. In some examples, the anti-CD70 CAR T cells may comprise a deletion in the TRAC gene relative to unmodified T cells. For example, the anti-CD70 CAR T cells may comprise a deletion of the fragment AGAGCAACAGTGCTGTGGCC (SEQ ID NO: 29) in the TRAC gene. This fragment can be replaced by the nucleic acid encoding the anti-CD70 CAR (e.g., SEQ ID NO: 154).

Alternatively or in addition, the population of anti-CD70 CAR T cells may comprise a disrupted β2M gene via CRISPR/Cas9 technology using the gRNA of β2M−1. Such anti-CD70 CAR T cells may comprise indels in the β2M gene, which comprise one or more of the nucleotide sequences of SEQ ID NOs: 57-62. See Table 3. In specific examples, anti-CD70 CAR T cells comprise ≥30% CARP T cells, ≤50% β2M+ cells, and ≤30% TCRαβ+ cells. In additional specific examples, anti-CD70 CAR T cells comprise ≥30% CARP T cells, ≤30% β2M+ cells, and ≤0.5% TCRαβ+ cells. See also WO 2019/097305A2, and WO2019215500, the relevant disclosures of each of which are incorporated by reference for the subject matter and purpose referenced herein.

### II. Use of Lenalidomide or Derivatives Thereof for Enhancing CAR-T Cell Productivity and Efficacy

The present disclosure reports that, unexpectedly, either in vitro or in vivo exposure of CAR-T cells to lenalidomide resulted in various advantageous features as disclosure herein without enhancing immune recognition of allogeneic CAR-T cells. Accordingly, some aspects of the present disclosure feature the use of lenalidomide or a derivative thereof for enhancing production and/or efficacy of CAR-T cells.

(i) Lenalidomide and Derivatives Thereof

Lenalidomide and its derivatives are small molecule compounds that modulates the substrate activity of the CRL4^{CRBN} E3 ubiquitin ligase. Lenalidomide has a structure of:

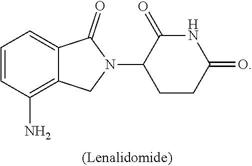 [SMILES: Nc1cccc2c1CN(C1CCC(=O)NC1=O)C2=O]

A lenalidomide derivative refers to a compound having the same core structure as lenalidomide and include one or more substitutions at one or more suitable positions as known to those skilled in the art. Suitable substituents include, but are not limited to, C₁₋₃ alkyl, halogen, —CN, —NO₂, —N₃, C₂₋₄ alkenyl, C₂₋₄ alkynyl, —NH₂, —OR or —SR, R being hydrogen, halogen, —CN, NO₂, —N₃, acyl, C₁₋₃ alkyl, C₂₋₄ alkenyl, or C₂₋₄ alkynyl. A lenalidomide derivative has substantially similar bioactivity as lenalidomide. Examples of lenalidomide derivatives include thalidomide and pomalidomide, the structures of which are provided below:

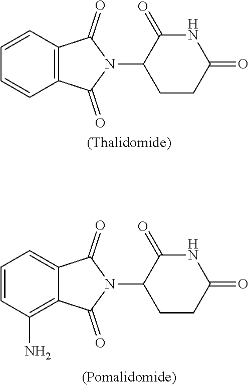 [SMILES: O=C1CCC(N2C(=O)c3ccccc3C2=O)C(=O)N1 ; Nc1cccc2c1C(=O)N(C1CCC(=O)NC1=O)C2=O]

(ii) Culturing CAR-T Cells with Lenalidomide or Derivatives Thereof In Vitro

In some aspects, provided herein are methods of using lenalidomide or a derivative thereof (e.g., those disclosed herein) in in vitro cell culture for producing CAR-T cells having improved features, for example, (i) enhanced T cell proliferation and/or expansion capacity; (ii) improved productivity, which may be reflected by increased T cell number; (iii) decreased senescence; (iv) improved effector activity, which optionally is characterized by improved cytokine secretion upon antigen stimulation; and/or improved cytotoxicity.

To perform the method disclosed herein, T cells such as CAR-T cells may be cultured in a medium comprising, among other components, lenalidomide or a derivative thereof under suitable conditions allowing for T cell growth and expansion. The T cells may be exposed to lenalidomide or the derivative thereof at any stage in a preparation process, for example, before genetic modification of the T cells, concurrently with genetic modification of the T cells, or after genetic modification of the T cells. In some examples, lenalidomide or a derivative thereof is used in culturing genetically modified CAR-T cells (e.g., those disclosed herein) for T cell expansion to produce the final CAR-T cell products.

A suitable amount of lenalidomide or the derivative thereof can be used in producing CAR-T cells to achieve one or more of the desired features of the resultant CAR-T cells. For example, about 0.1 μM to about 20 μM lenalidomide may be used in the methods disclosed herein. In some examples, about 0.3 μM to about 15 μM lenalidomide may be used. In other examples, about 0.5 μM to about 10 μM lenalidomide may be used. In some examples, about 0.1 μM, 0.2 μM, 0.3 μM, 0.4 μM. 0.5 μM, 0.6 μM, 0.7 μM, 0.8 μM, 0.9 μM. 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 15 μM, or 20 μM lenalidomide or a derivative thereof may be used in any of the methods disclosed herein.

T cells such as CAR-T cells may be cultured in the presence of lenalidomide or a derivative thereof for a suitable period, e.g., to maximize T cell expansion and growth, thereby obtaining a high number of CAR-T cells, which may be used in disease treatment. In some examples, the T cells such as CAR-T cells may be cultured in the presence of lenalidomide or a derivative thereof for about 5-about 30 days, e.g., about 5-10 days, about 10-15 days, about 15-20 days, or about 25-30 days.

In some examples, lenalidomide or a derivative thereof is used for producing anti-CD19 CAR-T cells such as those disclosed herein. For example, after TRAC and β2M knock-out and rAAV transduction to introduce the CAR-encoding nucleic acid, the resultant anti-CD19 CAR-T cells can be seeded at a suitable cell concentration for expansion. A suitable amount of lenalidomide or a derivative thereof (e.g., those disclosed herein) may be added to the culture medium. In some instances, the culture medium (comprising lenalidomide) may be replenished periodically (e.g., every 2-5 days such as every 3-4 days). Cell count and viability can also be monitored periodically (e.g., every 2-5 days such as every 3-4 days).

In some examples, lenalidomide or a derivative thereof is used for producing anti-BCMA CAR-T cells such as those disclosed herein. For example, after TRAC and β2M knock-out and rAAV transduction to introduce the CAR-encoding nucleic acid, the resultant anti-BCMA CAR-T cells can be seeded at a suitable cell concentration for expansion. A suitable amount of lenalidomide or a derivative thereof (e.g., those disclosed herein) may be added to the culture medium. In some instances, the culture medium (comprising lenalidomide) may be replenished periodically (e.g., every 2-5 days such as every 3-4 days). Cell count and viability can also be monitored periodically (e.g., every 2-5 days such as every 3-4 days).

In some examples, lenalidomide or a derivative thereof is used for producing anti-CD70 CAR-T cells such as those disclosed herein. For example, after TRAC, β2M, and CD70 knock-out and rAAV transduction to introduce the CAR-encoding nucleic acid, the resultant anti-CD70 CAR-T cells can be seeded at a suitable cell concentration for expansion. A suitable amount of lenalidomide or a derivative thereof (e.g., those disclosed herein) may be added to the culture medium. In some instances, the culture medium (comprising lenalidomide) may be replenished periodically (e.g., every 2-5 days such as every 3-4 days). Cell count and viability can also be monitored periodically (e.g., every 2-5 days such as every 3-4 days).

After the culturing, the CAR-T cells thus produced can be collected and be applied for therapeutic uses. In some instances, the CAR-T cells may be formulated in a pharmaceutical composition (e.g., as disclosed herein) and stored under suitable conditions for future use. In some examples, the CAR-T cells may be washed to remove lenalidomide or the derivative thereof. Alternatively, lenalidomide or the derivative thereof may be kept together with the CAR-T cells.

(iii) Therapeutic Applications

Any of the CAR-T cells produced by the methods disclosed herein (which is also within the scope of the present disclosure) can be used in disease treatment based on the binding activity of the CAR receptor expressed by the CAR-T cells.

In some aspects, the CAR-T cells may be formulated as pharmaceutical compositions comprising any of the CAR T cells as disclosed herein, for example, anti-CD19 CAR-T cells, anti-BCMA Car-T cells, or ant-CD70 CAR-T cells, and a pharmaceutically acceptable carrier. Such pharmaceutical compositions can be used in cancer treatment in human patients, which is also disclosed herein.

As used herein, the term “pharmaceutically acceptable” refers to those compounds, materials, compositions, and/or dosage forms which are, within the scope of sound medical judgment, suitable for use in contact with the tissues, organs, and/or bodily fluids of the subject without excessive toxicity, irritation, allergic response, or other problems or complications commensurate with a reasonable benefit/risk ratio. As used herein, the term “pharmaceutically acceptable carrier” refers to solvents, dispersion media, coatings, antibacterial agents, antifungal agents, isotonic and absorption delaying agents, or the like that are physiologically compatible. The compositions can include a pharmaceutically acceptable salt, e.g., an acid addition salt or a base addition salt. See, e.g., Berge et al., (1977) J Pharm Sci 66:1-19.

In some embodiments, the pharmaceutical composition further comprises a pharmaceutically acceptable salt. Non-limiting examples of pharmaceutically acceptable salts include acid addition salts (formed from a free amino group of a polypeptide with an inorganic acid (e.g., hydrochloric or phosphoric acids), or an organic acid such as acetic, tartaric, mandelic, or the like). In some embodiments, the salt formed with the free carboxyl groups is derived from an inorganic base (e.g., sodium, potassium, ammonium, calcium or ferric hydroxides), or an organic base such as isopropylamine, trimethylamine, 2-ethylamino ethanol, histidine, procaine, or the like).

In some embodiments, the pharmaceutical composition disclosed herein comprises a population of the genetically engineered CAR-T cells (e.g., those disclosed herein, for example, the anti-CD19 CAR-T cells, the anti-BCMA CAR-T cells, or the anti-CD70 CAR-T cells) suspended in a cryopreservation solution (e.g., CryoStor® C55). The cryopreservation solution for use in the present disclosure may also comprise adenosine, dextrose, dextran-40, lactobionic acid, sucrose, mannitol, a buffer agent such as N-)2-hydroxethyl) piperazine-N′-(2-ethanesulfonic acid) (HEPES), one or more salts (e.g., calcium chloride, magnesium chloride, potassium chloride, potassium bicarbonate, potassium phosphate, etc.), one or more base (e.g., sodium hydroxide, potassium hydroxide, etc.), or a combination thereof. Components of a cryopreservation solution may be dissolved in sterile water (injection quality). Any of the cryopreservation solution may be substantially free of serum (undetectable by routine methods).

In some instances, a pharmaceutical composition comprising a population of the CAR-T cells such as those disclosed herein can be suspended in a cryopreservation solution (e.g., substantially free of serum) and placed in storage vials.

The CAR-T cells or a pharmaceutical composition comprising such as disclosed herein can be administered to a subject for therapeutic purposes, for example, treatment of a cancer (e.g., a hematopoietic cancer or a solid tumor) targeted by the CAR construct expressed by the therapeutic CAR-T cells.

The step of administering may include the placement (e.g., transplantation) of the therapeutic CAR-T cells into a subject by a method or route that results in at least partial localization of the therapeutic T cells at a desired site, such as a tumor site, such that a desired effect(s) can be produced. Therapeutic T cells can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the lifetime of the subject, i.e., long-term engraftment. For example, in some aspects described herein, an effective amount of the therapeutic T cells can be administered via a systemic route of administration, such as an intraperitoneal or intravenous route.

In some embodiments, the CAR-T cells are administered systemically, which refers to the administration of a population of cells other than directly into a target site, tissue, or organ, such that it enters, instead, the subject's circulatory system and, thus, is subject to metabolism and other like processes. Suitable modes of administration include injection, infusion, instillation, or ingestion. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the route is intravenous.

A subject may be any subject for whom diagnosis, treatment, or therapy is desired. In some embodiments, the subject is a mammal. In some embodiments, the subject is a human. In some instances, the human patient has a cancer involving CD19⁺ cancer cells. CAR-T cells expressing an anti-CD19 CAR (e.g., disclosed herein) may be used to treat such a patient. In some instances, the human patient has a cancer involving BCMA⁺ cancer cells. CAR-T cells expressing an anti-BCMA CAR (e.g., disclosed herein) may be used to treat such a patient. In some instances, the human patient has a CD70⁺ solid tumor. CAR-T cells expressing an anti-CD70 CAR (e.g., disclosed herein) may be used to treat such a patient.

In some instances, the CAR-T cells can be allogeneic (syngeneic or xenogeneic) to the subject. “Allogeneic” means that the therapeutic T cells are not derived from the subject who receives the treatment but from different individuals (donors) of the same species as the subject. A donor is an individual who is not the subject being treated. A donor is an individual who is not the patient. In some embodiments, a donor is an individual who does not have or is not suspected of having the cancer being treated. In some embodiments, multiple donors, e.g., two or more donors, are used.

In some embodiments, the CAR-T cells being administered according to the methods described herein comprises allogeneic T cells obtained from one or more donors. Such allogeneic CAR-T cells may be derived from immune cells obtained from one or more different donors of the same species, where the genes at one or more loci are not identical to the recipient (e.g., subject). For example, the CAR-T cells being administered to a subject can be derived from one or more unrelated donors, or from one or more non-identical siblings. In some embodiments, syngeneic cell populations may be used, such as those obtained from genetically identical donors, (e.g., identical twins). In some embodiments, the cells are autologous cells; that is, the engineered T cells are obtained or isolated from a subject and administered to the same subject, i.e., the donor and recipient are the same. In some examples, the CAR-T cells disclosed herein are derived from immune cells obtained from one or more healthy human donors.

An effective amount refers to the amount of a population of engineered T cells needed to prevent or alleviate at least one or more signs or symptoms of a medical condition (e.g., cancer), and relates to a sufficient amount of a composition to provide the desired effect, e.g., to treat a subject having a medical condition. An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

The efficacy of a treatment using the therapeutic T cells disclosed herein can be determined by the skilled clinician. A treatment is considered “effective”, if any one or all of the signs or symptoms of, as but one example, levels of functional target are altered in a beneficial manner (e.g., increased by at least 10%), or other clinically accepted symptoms or markers of disease (e.g., cancer) are improved or ameliorated. Efficacy can also be measured by failure of a subject to worsen as assessed by hospitalization or need for medical interventions (e.g., progression of the disease is halted or at least slowed). Methods of measuring these indicators are known to those of skill in the art and/or described herein. Treatment includes any treatment of a disease in subject and includes: (1) inhibiting the disease, e.g., arresting, or slowing the progression of symptoms; or (2) relieving the disease, e.g., causing regression of symptoms; and (3) preventing or reducing the likelihood of the development of symptoms.

(iv) Combined Therapy of CAR-T Cells and Lenalidomide or Derivatives Thereof

CAR-T cells, either exposed to lenalidomide or not exposed to lenalidomide in vitro, may be co-used other therapeutic agents, for treating the same indication, or for enhancing efficacy of the therapeutic T cells and/or reducing side effects of the therapeutic T cells.

In some aspects, provided herein are combined therapies comprising any of the CAR-T cells disclosed herein (with or without in vitro exposure to lenalidomide) and lenalidomide or a derivative thereof.

In some examples, the treatment involving the CAR-T cells and the treatment involving lenalidomide or the derivative thereof may be performed sequentially. For example, a subject in need of the treatment may receive the CAR-T cells first and then subject to the treatment involving lenalidomide or its derivative within a suitable time period. Alternatively, subject may complete a course of treatment comprising lenalidomide or its derivative and then followed by a treatment comprising the CAR-T cells.

Alternatively, the treatment involving the CAR-T cells and the treatment involving lenalidomide or the derivative thereof may be concurrent. For example, a subject may start the treatment of lenalidomide or its derivative on a daily basis. After receiving one or more daily doses of lenalidomide or a derivative thereof, the patient may be administered with CAR-T cells. Treatment with lenalidomide or its derivative may continue after administration of the CAR-T cells.

In some examples, the subject is a human patient having a CD19⁺ cancer. Such a human patient can be subject to a combined therapy comprising anti-CD19 CAR-T cells (e.g., those disclosed herein) and lenalidomide or a derivative thereof.

In some examples, the subject is a human patient having a BCMA⁺ cancer. Such a human patient can be subject to a combined therapy comprising anti-BCMA CAR-T cells (e.g., those disclosed herein) and lenalidomide or a derivative thereof.

In some examples, the subject is a human patient having a CD70⁺ cancer. Such a human patient can be subject to a combined therapy comprising anti-CD70 CAR-T cells (e.g., those disclosed herein) and lenalidomide or a derivative thereof.

### III. Kit for Production and Therapeutic Uses of CAR-T Cells

The present disclosure also provides kits for use in producing the CAR-T cells disclosed herein and for their therapeutic uses.

In some embodiments, a kit provided herein may comprise components for performing genetic edit of the one or more targeting genes disclosed herein, including TRAC gene, β2M gene, CD70 gene, TGFBRII gene, TET2 gene, and/or Reg1 gene. The kit may also comprise a population of immune cells to which the genetic editing will be performed (e.g., a leukopak or a T cell bank). The components for genetically editing one or more of the target genes may comprise a suitable endonuclease such as an RNA-guided endonuclease and one or more nucleic acid guides, which direct cleavage of one or more suitable genomic sites by the endonuclease. For example, the kit may comprise a Cas enzyme such as Cas 9 and one or more gRNAs targeting the one or more target genes. Any of the gRNAs specific to these target genes (e.g., those provided in Table 1 below) can be included in the kit.

In some embodiments, a kit provided herein may comprise one or more components for producing CAR-T cells as also disclosed herein. Such components may comprise an endonuclease suitable for gene editing and a nucleic acid coding for a CAR construct of interest. The CAR-coding nucleic acid may be part of a donor template as disclosed herein, which may contain homologous arms flanking the CAR-coding sequence. In some instances, the donor template may be carried by a viral vector such as an AAV vector.

Further, the kit comprises lenalidomide or a derivative thereof for use in in vitro culture of the CAR-T cells as disclosed herein.

Any of the kit disclosed herein may further comprise instructions for making the therapeutic T cells, or therapeutic applications of the therapeutic T cells. In some examples, the included instructions may comprise a description of using the gene editing components to genetically engineer one or more of the target genes (e.g., TRAC, β2M, CD70, TGFBRII, TET2, Reg1, or a combination thereof). In other examples, the included instructions may comprise a description of how to introduce a nucleic acid encoding a CAR construction into the T cells and how to use lenalidomide or its derivative for making therapeutic T cells.

In yet other embodiments, the kit disclosed herein may comprise a population of CAR-T cells as disclosed for the intended therapeutic purposes. Such a kit may comprise a population of genetically engineered T cells (e.g., CAR-T cells) for use to eliminate undesired cells targeted by the CAR construct (e.g., for treatment of cancer such as a solid tumor). Such a kit may comprise one or more containers in which the genetically engineered T cells can be placed.

The kit may further comprise instructions for administration of the therapeutic T cells as disclosed herein to achieve the intended activity, e.g., eliminating disease cells targeted by the CAR expressed on the therapeutic T cells. Alternatively or in addition, the kit may further comprise a description of selecting a subject suitable for treatment based on identifying whether the subject is in need of the treatment. The instructions relating to the use of the therapeutic T cells described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the therapeutic T cells are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an infusion device for administration of the therapeutic T cells. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds.(1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Lenalidomide Showed Beneficial Effect on Multiple Aspects of BCMA Directed CAR-T Cells In-Vitro

Anti-BCMA CAR-T cells were used in this Example as exemplary CAR-T cell. The anti-BCMA CAR-T cells express an anti-BCMA CAR comprising the amino acid sequence of SEQ ID NO: 131, a disrupted TRAC gene having the anti-BCMA CAR coding sequence inserted, and a disrupted β2M gene.

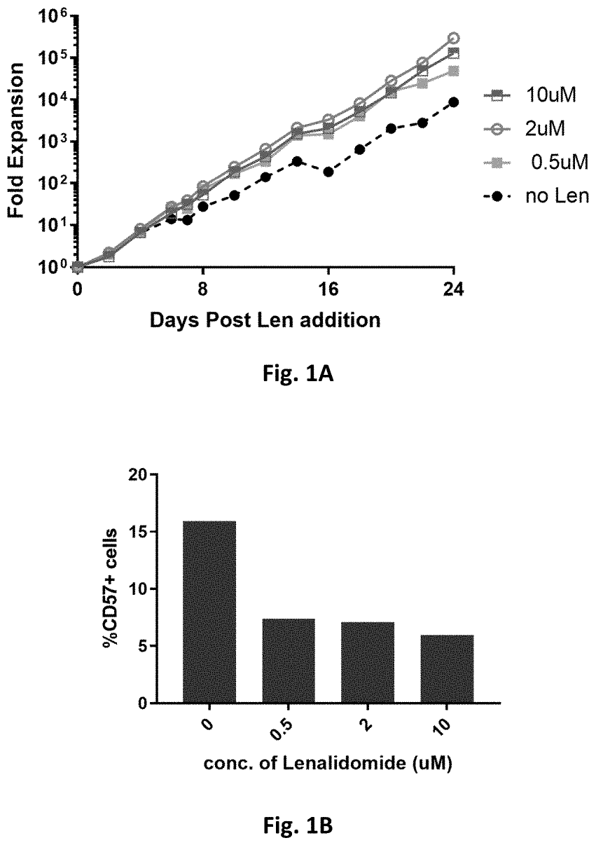

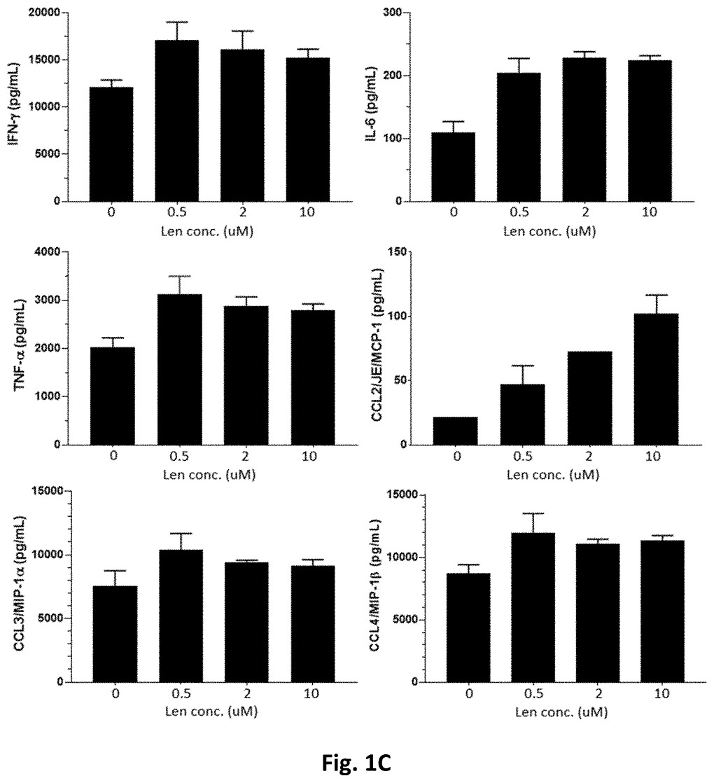

The CAR-T cells were thawed and expanded in-vitro in the presence or absence of Lenalidomide. Multiple concentrations of Lenalidomide were added to the culture media, to evaluate the activity of Lenalidomide across a wide range of concentrations, from 0.5 uM to 10 uM. In all tested concentrations, Lenalidomide enhanced the proliferation of the anti-BCMA CAR-T cells, showing 5-30 fold higher expansion in the tested time period (FIG. 1A). The anti-BCMA CAR-T cells expanded in the presence of Lenalidomide showed decreased senescence as evident by reduced expression of CD57 in the cell population in all the tested concentrations of Lenalidomide (FIG. 1B, tested after 10 day culture with Lenalidomide).

In addition to enhancing CAR-T cell expansion, Lenalidomide enhanced effector cytokine secretion upon antigen stimulation in all the Lenalidomide concentrations tested. FIG. 1C shows the level of multiple cytokines following an overnight culture of the anti-BCMA CAR-T cells with a cell line which expresses low levels of BCMA (JeKo-1), at a ratio of 2:1 effector to target cell. Addition of Lenalidomide to the co-culture media led to enhanced cytokine secretion of multiple effector cytokines, among them IFN-γ and TNF-α following CAR-T cell engagement by the BCMA expressing target cell line (FIG. 1C).

Lenalidomide was also found to enhance cell expansion and viability of CAR-T cells expressing CAR targeting various antigens, for example, anti-CD19 CAR and anti-CD70 CAR.

### Example 2: Lenalidomide Enhanced BCMA Directed CAR-T Cell Activity In-Vivo in Mice

The effect of a combination treatment of the anti-BCMA CAR-T cells described in Example 1 above and Lenalidomide was tested in mice using an MM.1S subcutaneous tumor model. Mice were inoculated with MM.1S cells, and the tumor was allowed to reach a mean volume of 150 mm³. Once tumors reached target volume, mice were treated with:

a) 3 million anti-BCMA CAR-T cells,

b) Lenalidomide at a dose of 1.5 mg/kg daily for 21 days, followed by 3 days off and QD4 till end,

c) Lenalidomide at a dose of 10 mg/kg daily for 14 days, followed by 3 days off and QD4 till end,

d) combination of anti-BCMA CAR-T cells and Lenalidomide at a dose of 1.5 mg/kg using the schedule described in b, or

e) combination of anti-BCMA CAR-T cells and Lenalidomide at a dose of 10 mg/kg using the schedule described in c.

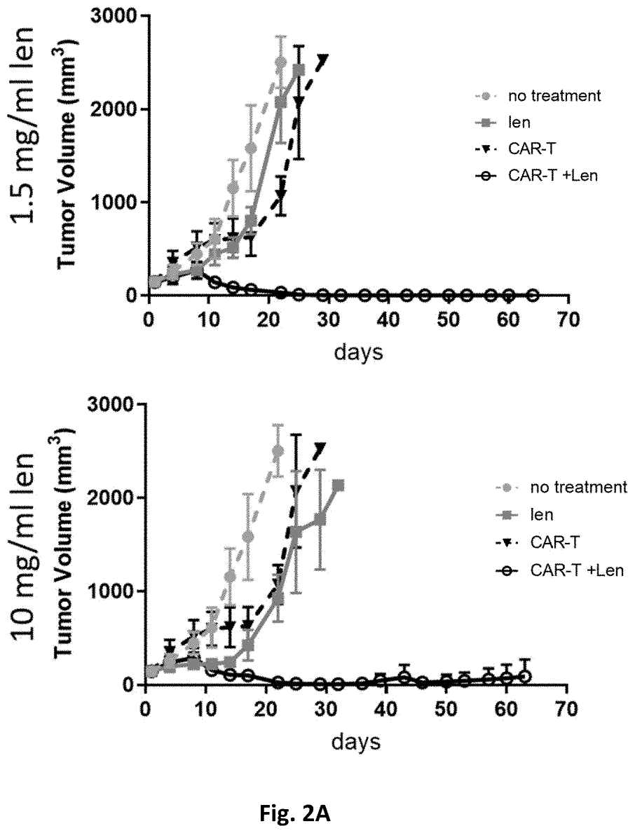

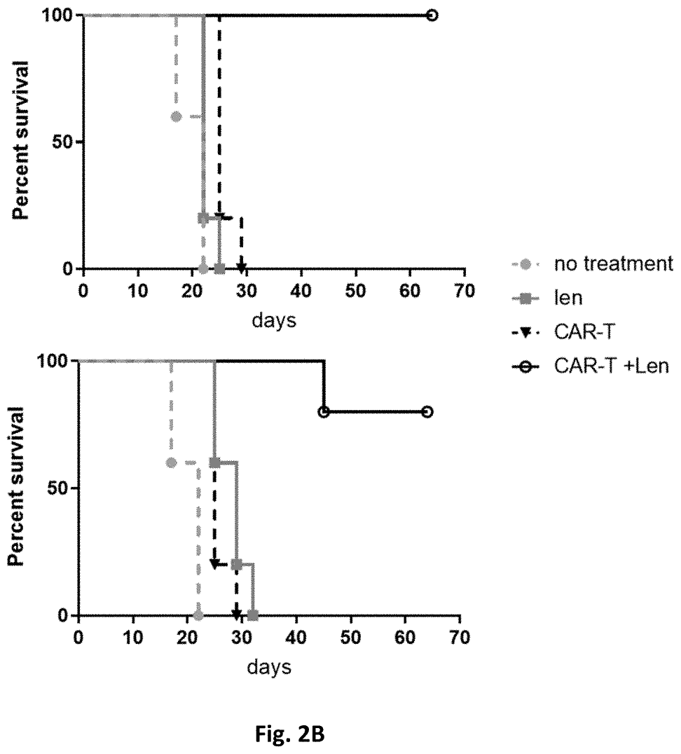

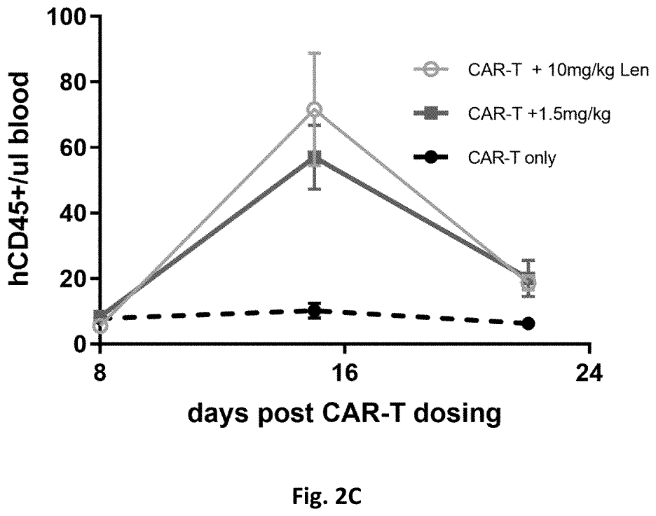

The effect of each treatment on tumor regression and mouse survival was monitored throughout the study. Single arm treatment of either the anti-BCMA CAR-T cells or Lenalidomide in both tested doses showed a minimal effect on both tumor regression and mouse survival compared to the no treatment arm. However, the combination arm showed a potent inhibition of tumor growth in both Lenalidomide doses tested, with complete tumor clearance of 5/5 mice in the low Lenalidomide dose, and 4/5 in the higher lenalidomide dose (FIG. 2A). This led to prolonged mouse survival in the combination arm, and while in the single treatment arms all mice were sacrificed due to reaching max tumor volume by day 32, in the combination arms 5/5 mice survived at day 64 in the low Lenalidomide dose, and 4/5 mice survived in the high Lenalidomide dose at day 64. FIG. 2B.

Examination of the anti-BCMA CAR-T cells expansion in peripheral blood, revealed that co-administration of Lenalidomide enhanced the expansion of the CAR-T cells following dosing in mice. Presence of human cells in mouse blood was evaluated using staining for human CD45+, and the number of human cells per ul of mouse blood was calculated using BD TruCount vials per manufacturer's protocol. Human T cells were quantified in mouse blood ˜1, 2 & 3 weeks after the CAR-T cells dosing to mice. Lenalidomide was found to significantly increase the numbers of the CAR-T cells in mouse blood in a dose dependent manner, 2 & 3 weeks after CAR-T dosing, with maximal increase from 10 cells/ul in the absence of Lenalidomide to ˜70 cells/ul in the presence of 10 mg/kg Lenalidomide, 2 weeks post dosing (FIG. 2C).

In a human clinical trial for treatment of multiple melanoma, it was observed that, at equivalent dose levels, lenalidomide deepened NK cell depletion and delayed recovery of NK cells in patients treated with both anti-BCMA CAR-T cells and lenalidomide, as compared with patients treated with the anti-BCMA CAR-T cell alone. Lenalidomide also resulted in extended lymphocyte suppression in patients treated with both anti-BCMA CAR-T cells and lenalidomide, as compared with patients treated with the anti-BCMA CAR-T cell alone. Further, lenalidomide resulted in a higher level of circulating CAR-T cells in patients treated with both anti-BCMA CAR-T cells and lenalidomide, as compared with patients treated with the anti-BCMA CAR-T cell alone. Preliminary results from human clinical trials also suggest that, at equivalent dose levels, patients receiving lenalidomide in combination with anti-BCMA CAR-T cells exhibited increased anti-myeloma activity as compared with the monotherapy of the anti-BCMA CAR-T cells.

### Example 3: Lenalidomide Did not Enhance Immune Recognition of Allogenic T Cells

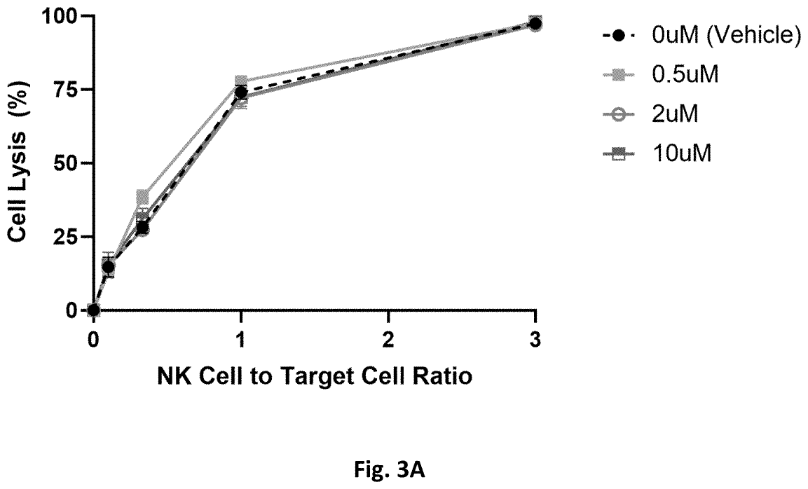

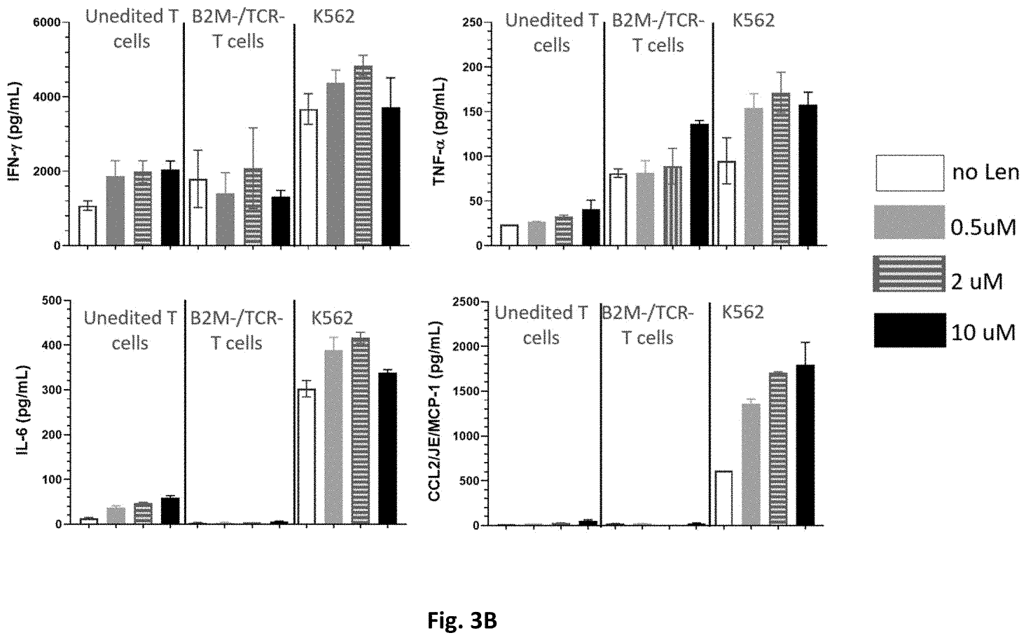

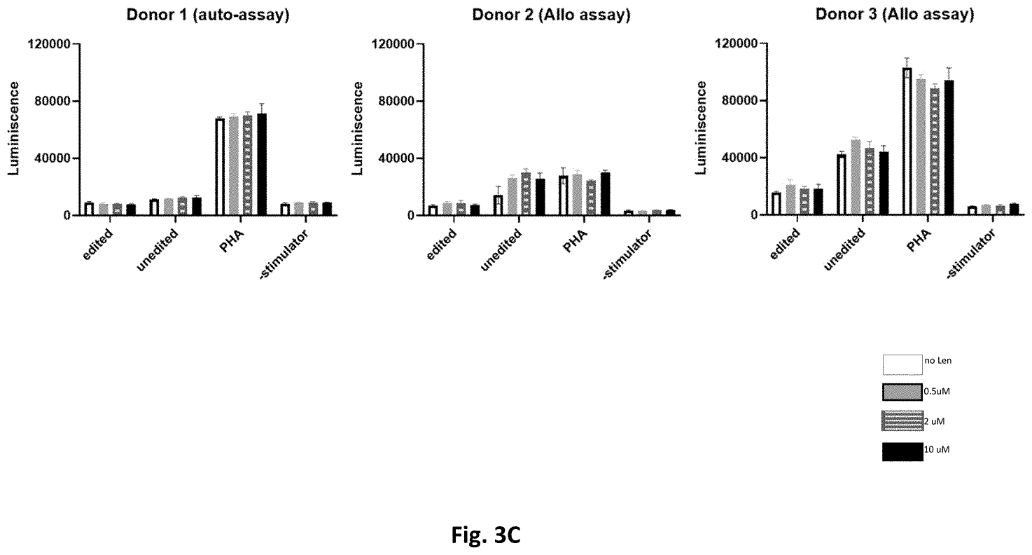

Since Lenalidomide has been shown to have a co-stimulatory effect on T cells, and stimulate NK cells, the ability of allogenic T cells (B2M−/TRAC− cells) to stimulate immune recognition of allogenic cells was assessed. Two modes of allogenic immune recognition were tested: immune recognition of B2M^{neg} cells by NK cells, and immune recognition by allogenic T cells. Examination of the cytotoxic activity of NK cells towards B2M^{neg} cells was tested following overnight (ON) co-culture in varying concentrations of NK to T cells, and varying concentrations of Lenalidomide. Increasing concentration of NK to B2M^{neg} T cells led to an increase in the cytotoxic activity of NK cells towards B2M^{neg} T cells. Surprisingly, adding Lenalidomide in a wide range of concentrations did not lead to an increased cell killing of the B2M^{neg} T cells (FIG. 3A).

Additionally, cytokine secretion was tested at the end of the co-culture described above, following co-culture of NK cells with B2M^{neg} T cells, K562 cells (a B2M^{neg} cell line, commonly used as a positive control for activation of NK cells due to lack of B2M expression), and unedited T cells (used as a negative control for NK cells activation). Analysis of cytokine secretion following co-culture with NK cells, showed that several cytokines were upregulated upon co-culture of NK cells with K562 cells. This included cytokines previously shown to be upregulated upon NK cell activation, such as IL-6, MCP-1, IFN-γ and TNF-α. Upregulation of secretion of several cytokines has been observed upon addition of Lenalidomide to the co-culture of NK cell with K562, which is consistent with the known role of Lenalidomide in enhancing NK cell activation (FIG. 3B). However, when examining the cytokine secretion upon co-culture of NK cells with B2M^{neg} T cells, the levels of several cytokines were much lower compared to the co-culture with K562, and minimal changes were observed upon addition of Lenalidomide, in the concentrations tested (FIG. 3B). This indicated that, although in some case cytotoxic activity of NK cells can be enhanced in the presence of Lenalidomide, enhanced NK recognition of allo T cells does not seem to be a concern, following addition of Lenalidomide.

Next, allo-reactivity towards edited T cells (B2M^{neg}/TCR^{neg}) was tested using an MLR assay (mixed lymphocyte reaction). In this assay PBMCs (“responder cells”) were mixed with irradiated auto or allo T cells (“stimulator cells”). At the end of the assay, the activation of the responder cells was evaluated by measuring the cell proliferation in the co-culture, with cell proliferation serving as a proxy for immune activation. In the assay shown in FIG. 3C, both auto (donor 1) & allo (donor 2 & 3) were evaluated for immune activation following co-culture. As shown in FIG. 3C, in the allo setting, proliferation was observed upon co-culture of unedited T cells with PBMCs from 2 individual donors. As expected, immune activation was reduced upon deletion of B2M & TRAC from the T cells, as evident by the reduced proliferation in both donors tested. Addition of Lenalidomide may in some cases enhance allo reactivity towards unedited T cells (see donor 2 panel FIG. 3C, unedited T cells, in the various Lenalidomide concentrations tested). However, the proliferation observed upon allo co-culture with edited T cells remained low, with minimal changes upon addition of Lenalidomide to the co-culture, indicating that the allo reactivity towards edited T cells was unaffected by the addition of Lenalidomide.

Taken together, results from this Example show that, unexpectedly, Lenalidomide did not enhance immune recognition of allogenic T cells.

### Example 4: BCMA Directed CAR-T Cells Produced in the Presence of Lenalidomide Exhibited Increased Cytokine Secretion Upon Antigen Stimulation

PBMCs were thawed and activated by T cell activation agents to enrich for T cells. After 3 days, T cells were edited for B2M and TRAC knock-out using a CRISPR/Cas gene editing system. An anti-BCMA expression cassette (as an exemplary CAR construct) was knocked into the TRAC locus to produce anti-BCMA CAR-T cells. Following the editing procedure, resulting T cells were expanded in the presence of absence of Lenalidomide in a concentration of 0.5, 2, & 10 uM for approximately 10 days. The resulting cells were later evaluated for cytokine secretion following antigen stimulation, in the absence of Lenalidomide.

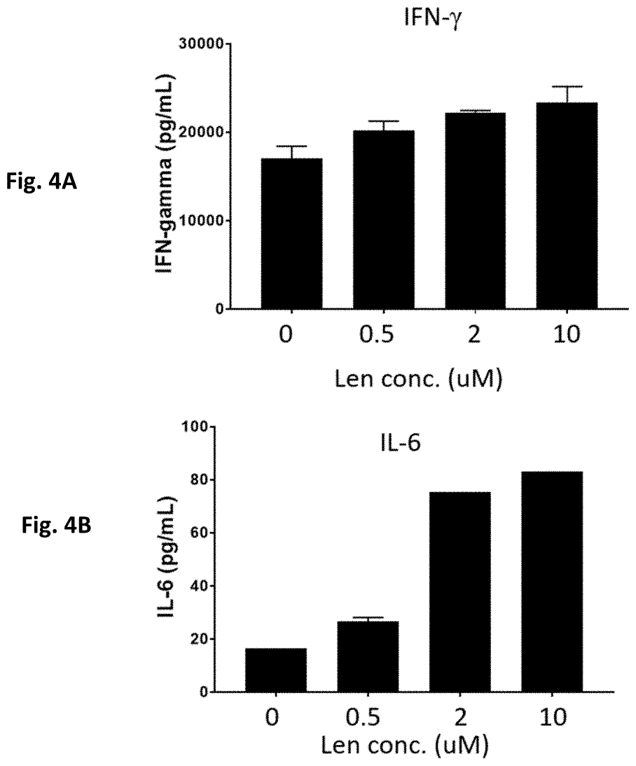

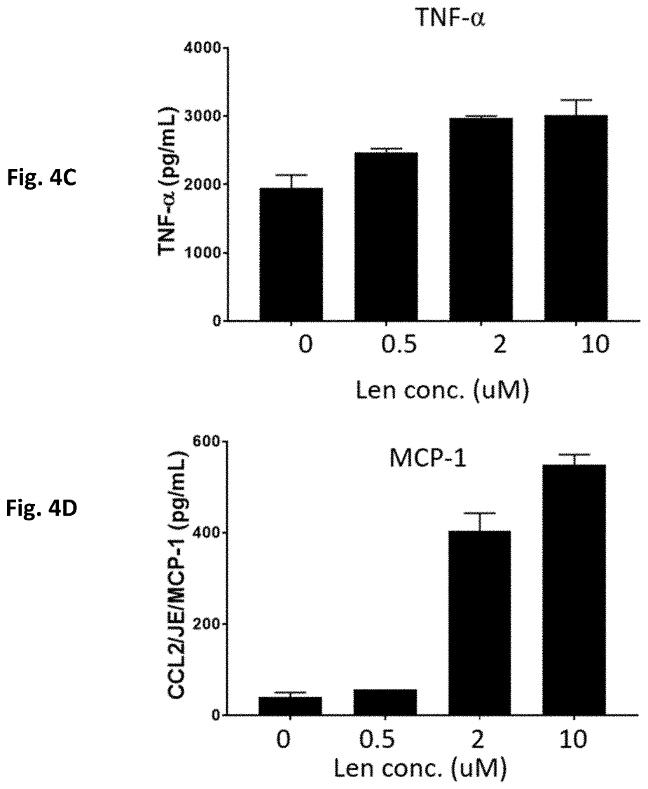

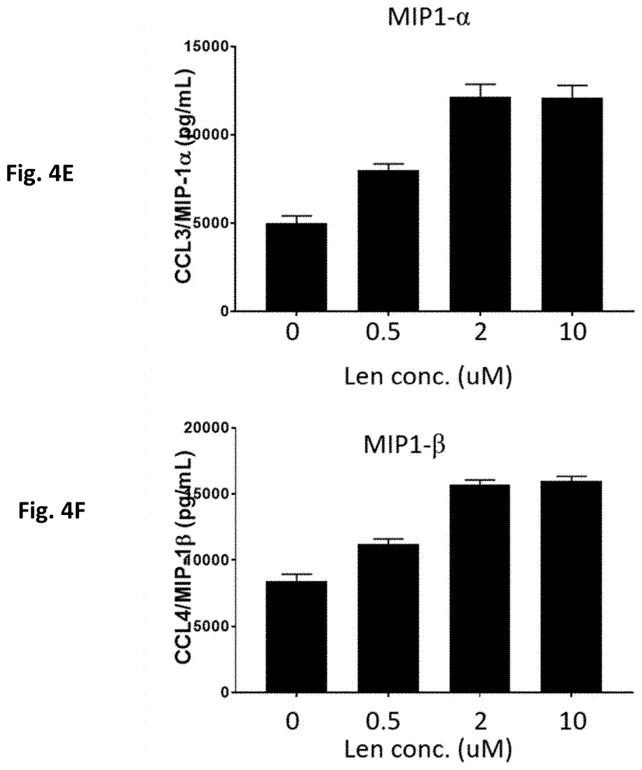

Lenalidomide addition during production of the anti-BCMA CAR-T cells was found to enhance effector cytokine secretion upon antigen stimulation, in the absence of continued presence of Lenalidomide. FIGS. 4A-4F shows the level of multiple cytokines following an overnight culture of the CAR-T cells with a cell line which expresses low levels of BCMA (JeKo-1), at a ratio of 0.5:1 effector to target cell. The inclusion of Lenalidomide to the co-culture media led to enhanced cytokine secretion of multiple effector cytokines, among them IFN-γ and TNF-α, upon CAR-T engagement by the BCMA expressing target cell line (FIGS. 4A-4F). This indicated that inclusion of Lenalidomide during the manufacturing process could serve as a means to not only enhance CAR-T cell proliferation, but also enhance the potency of the CAR-T cells, by programming them to a state with enhanced cytokine secretion upon antigen engagement.

### Example 5: Impact of Lenalidomide on CAR-T Cell Features

This Example investigates the effects of Lenalidomide on various CAR-T features.

Editing Efficiency

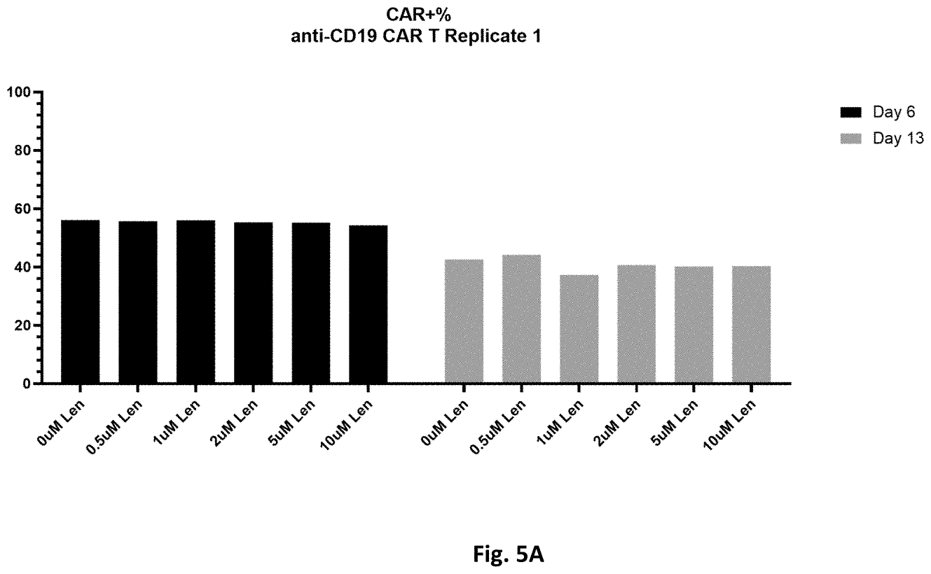

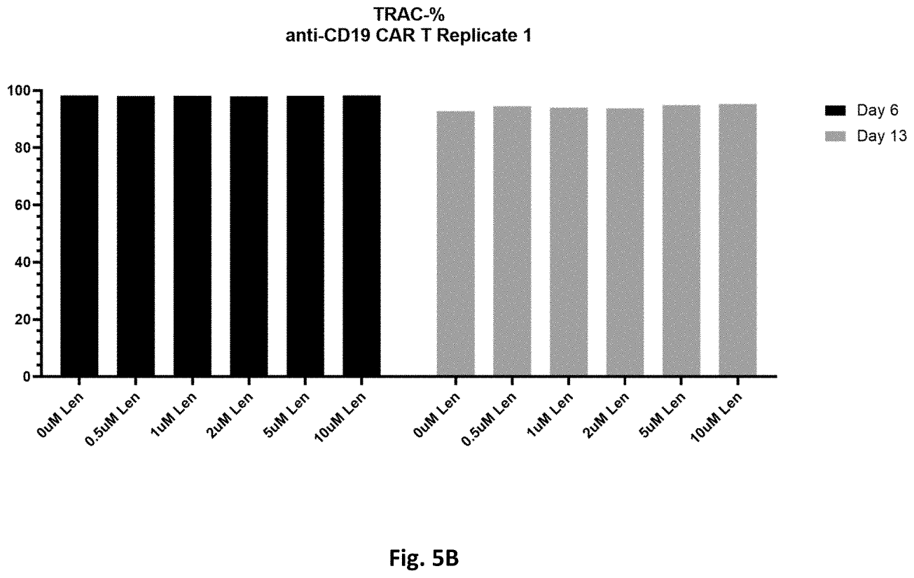

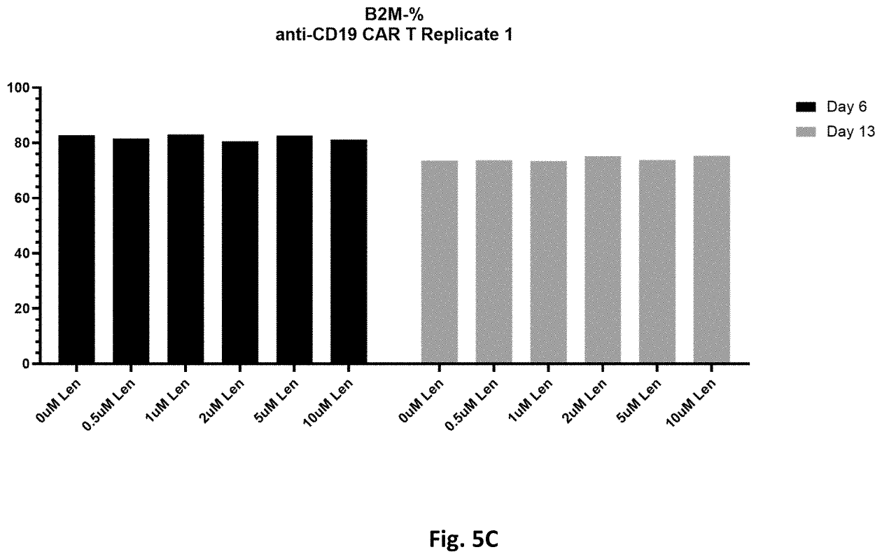

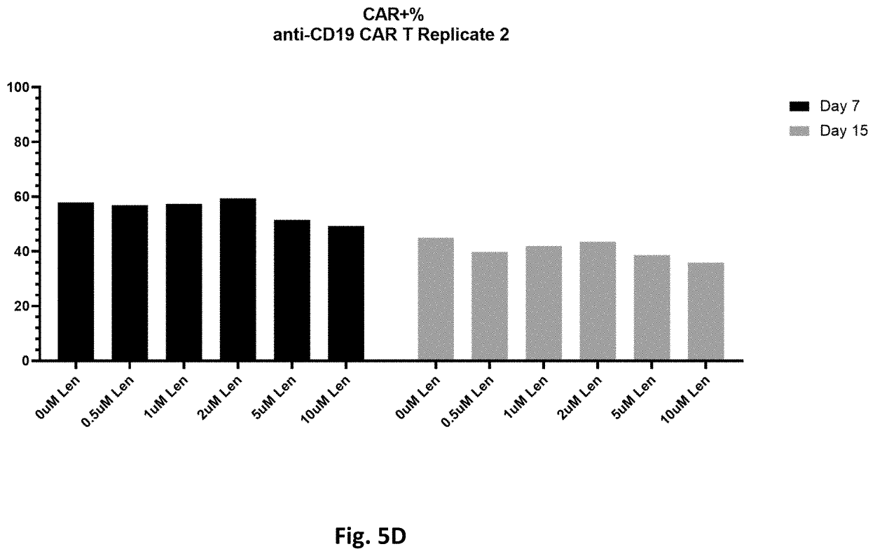

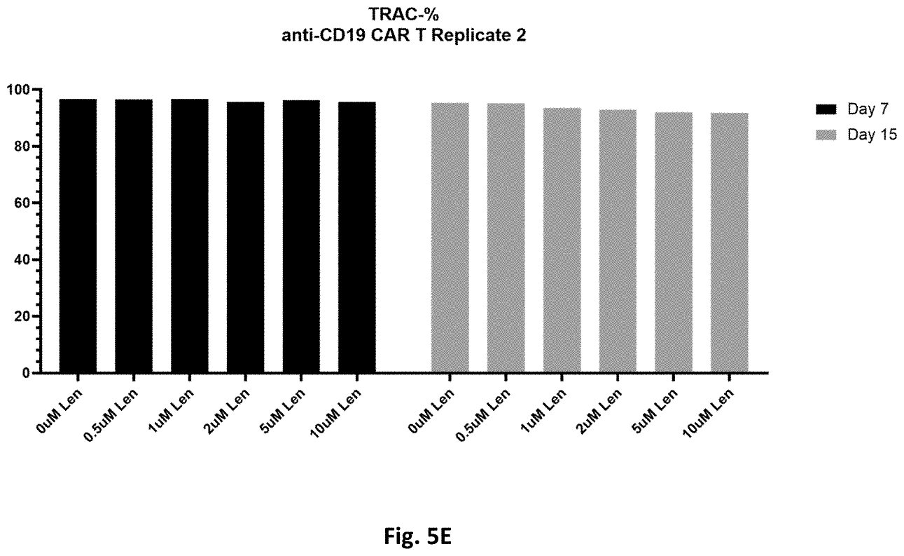

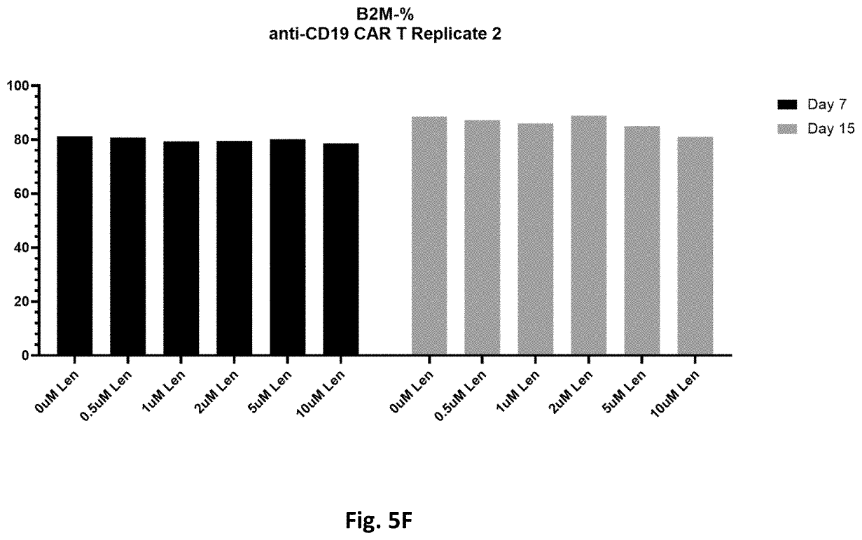

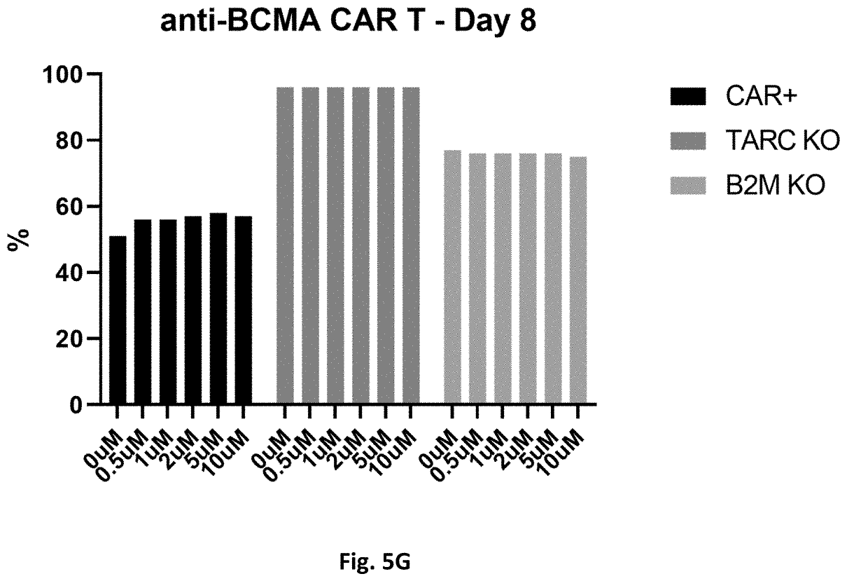

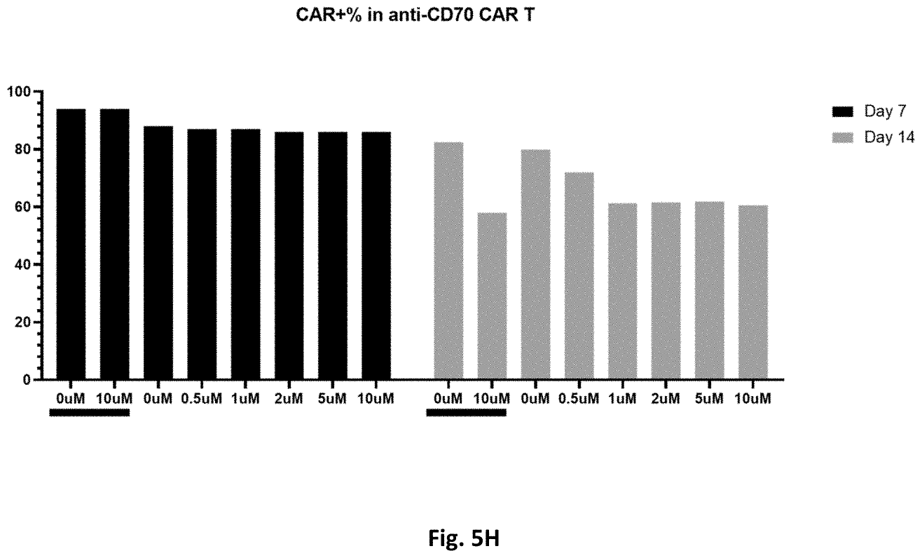

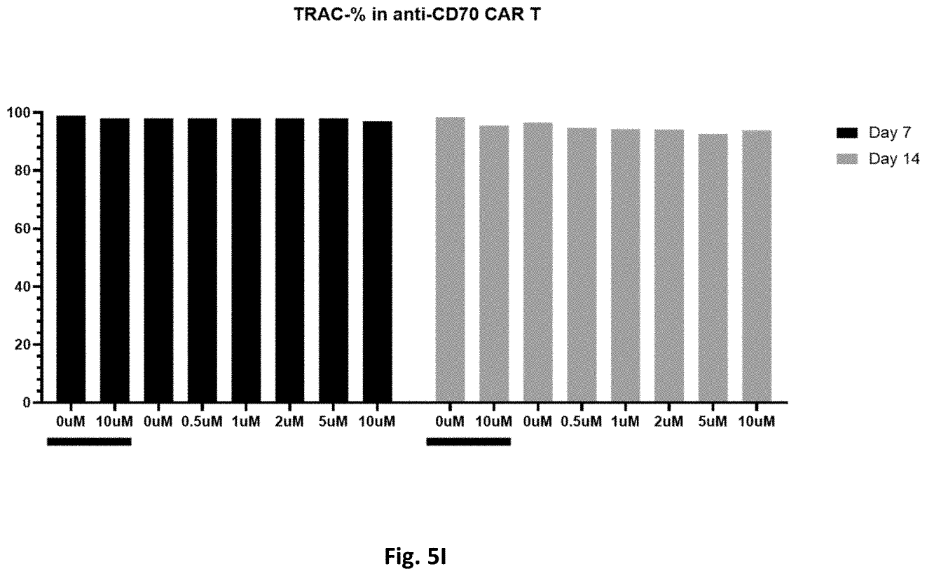

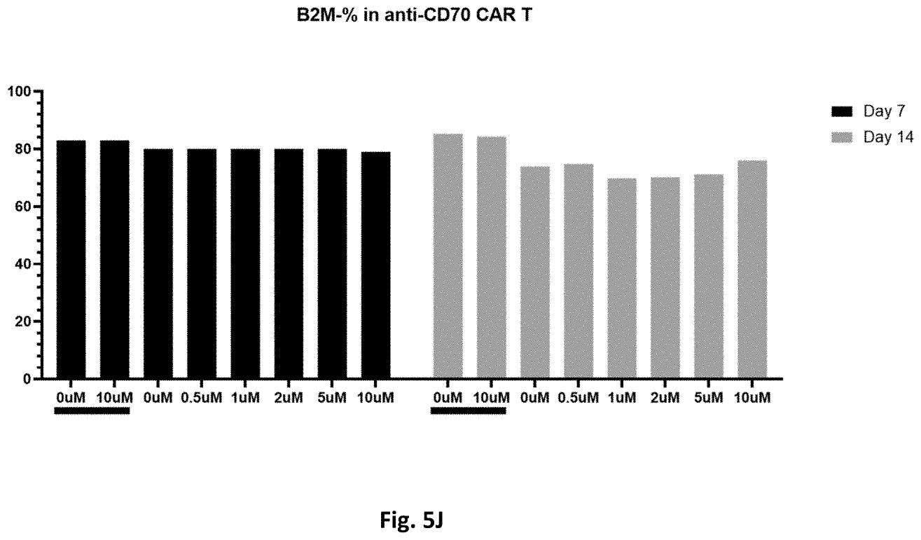

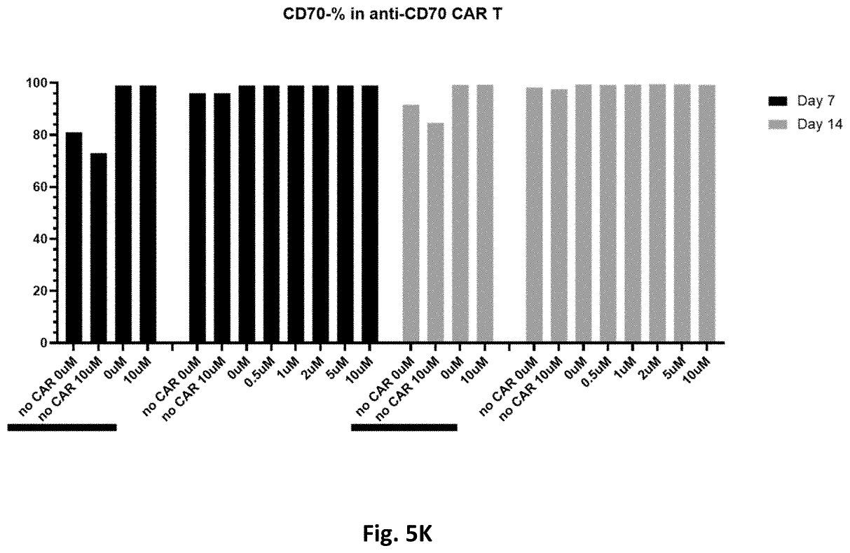

Editing efficiency, including TRAC-%, B2M-% and CAR+% were assessed at day 7/8 and/or day 13/14 with anti-CD19 CAR-T cells, anti-BCMA CAR-T cells, or anti-CD70 CAR-T cells. FIGS. 5A-5F show the CAR+%, TRAC-% and B2M-% from anti-CD19 CAR-T cells from two independent studies. About 50%-60% CAR+ cells were seen in the anti-CD19 CAR-T cells. Lenalidomide did not significantly alter CAR+%. CAR+% on day 13 or 15 was slightly decreased from day 6 and 7 but remain ≥30%. TRAC-% was above 90% on Lenalidomide treatment or non-treated cells on both day 6/7 and day 13/15. About 80% B2M-% was seen in both studies, without significant changes upon lenalidomide treatment on both day 6/7 and day 13/15.

FIG. 5G shows the CAR+%, TRAC-% and B2M-% from the anti-BCMA CAR-T cells on day 8. Anti-BCMA CAR-T cells were not harvested around day 14 due to slower growth rate. About 51-58% of CAR+%, 96% TRAC-% and 75-77% of B2M-% were detected from anti-BCMA CAR-T cells with or without Lenalidomide treatment.

FIGS. 5H-5K show the CAR+%, TRAC-%, B2M-% and CD70-%. CD70 electroporation was performed on the day 0 (T cell thawing day) for anti-CD70 CAR-T cell Process 1, but on day 1 (24 hr post activation) for anti-CD70 CAR-T cell Process 2. CD70-% on CD70 electroporated cells without CD70 CAR on day 7 increased from 81% to 96% and increased from 91.7% to 98.3% on day 14 if electroporation was performed after 24 hr's activation. Lenalidomide decreased CD70-% on anti-CD70 CAR-T electroporated cells (Process 1) by about 7% but not on anti-CD70 CAR-T electroporated cells (process 2). CAR+% from anti-CD70 CAR-T cells (Process 2) was 88% on day 7 and 80% on day 14 (FIG. 5H). CAR+% from anti-CD70 CAR-T cells (Process 1) was 94% on day 7 and 82.5% on day 14. (FIG. 5H). TRAC-% of anti-CD70 CAR-T cells (from both processes) were around 97-98% on day 7 and 93%-98% on day 14 (FIG. 5I). B2M-% of the anti-CD70 CAR-T cells from Process 1 and Process 2 were around 80-83% on day 7 and 70%-85% on day 14 (FIG. 5J). No significant changes of TRAC-% and B2M-% was observed upon Lenalidomide's treatment.

CD4 and CD8 Ratio

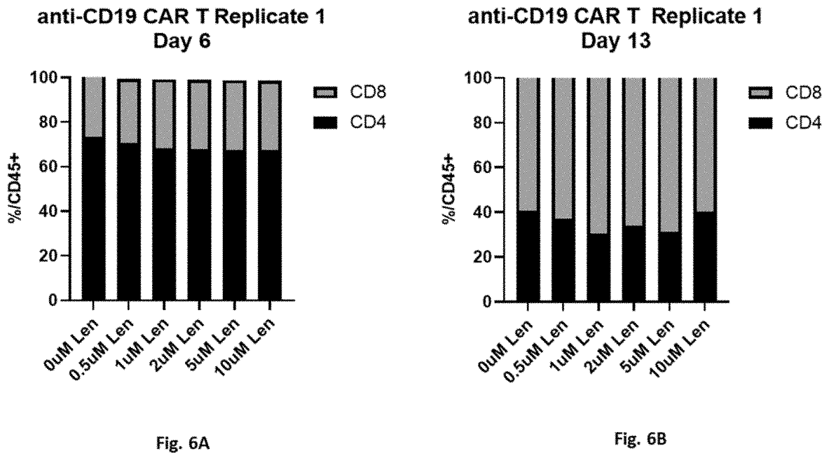

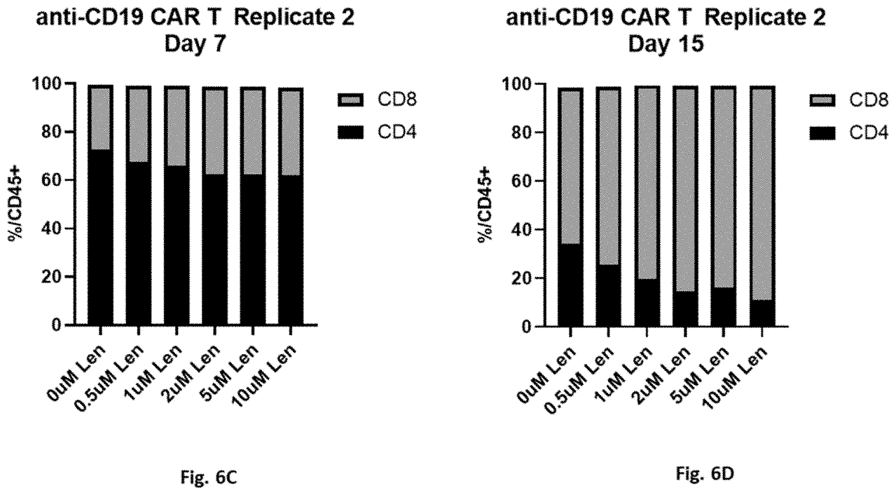

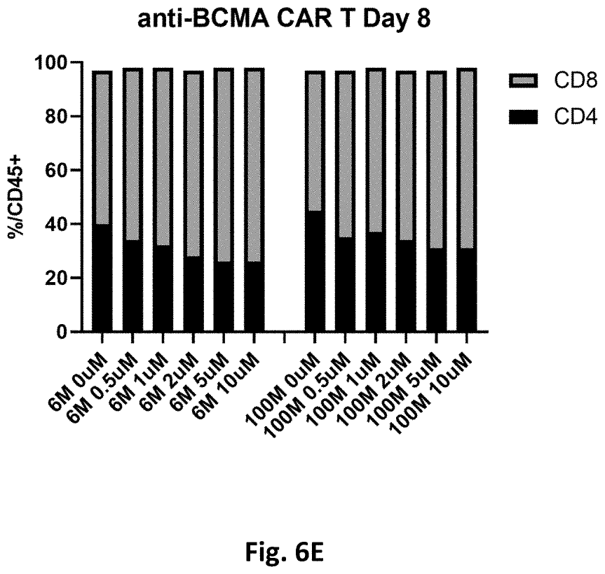

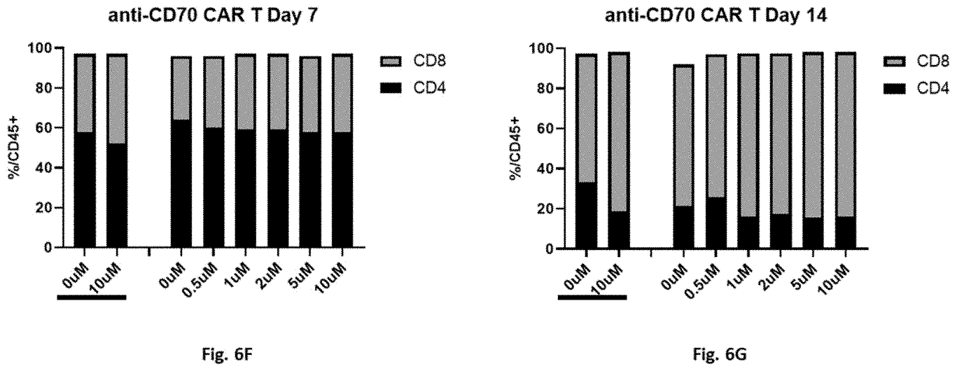

CD4% and CD8% were assessed at day 7/8 and/or day 13/14 with the anti-CD19, anti-BCMA, and anti-CD70 CAR-T cells disclosed above. FIGS. 6A-6D show CD4% and CD8% from the anti-CD19 CAR-T cells from two independent studies on day 6/7 and day 13/15. Both studies demonstrated dominant CD4 populations on day 6 or day 7, which were replaced by CD8+ cells on day 13 and day 15. The screwing of CD8+ cells were not significantly impacted by Lenalidomide treatment on day 6/7 and day 13 with study #1. There was about 10%-14% increase of CD8+ cells with lenalidomide treated cells on day 15 with study #2.

FIG. 6E shows CD4% and CD8% from the anti-BCMA CAR-T cells expanded at small and medium scale on day 8. The anti-BCMA CAR-T cells were not assessed and harvested around day 14 due to slower expansion. Compared with Lenalidomide untreated anti-BCMA CAR-T cells, there was dose-dependent increase of CD8 positive cells, ranging from 7-15%. However, the overall distribution of CD4 and CD8 cells was not significantly altered. Expansion scale (small or medium) didn't impact the CD4 and CD8 phonotype.

FIGS. 6F-6G show CD4% and CD8% from anti-CD70 CAR T cells. Compared with untreated anti-CD70 CAR-T cells, Lenalidomide treatment didn't change CD4+% and CD8+% on day 8 and day 15. Similar to the anti-CD19 CAR-T cells, there was skewing of CD8+ cells at day 15, which was independent from the timing of CD70 electroporation and Lenalidomide treatment.

### Example 6: In Vitro Cytotoxicity of CAR-T Cells Cultured with Lenalidomide

The ability of the anti-CD19, anti-BCMA, and anti-CD70 CAR-T cells disclosed herein, cultured with or without Lenalidomide to kill antigen-positive positive target cells, was assessed using a flow cytometry-based cytotoxicity assay. CD19+ Raji cell line was used for the anti-CD19 CAR-T cells, a BCMA+ cell line was used for the anti-BCMA CAR-T cells, and a CD70+ cell line was used for the anti-CD70 CAR-T cells.

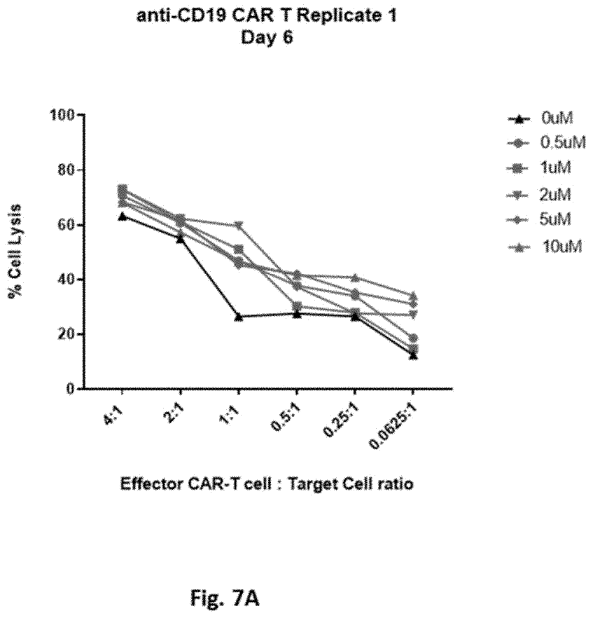

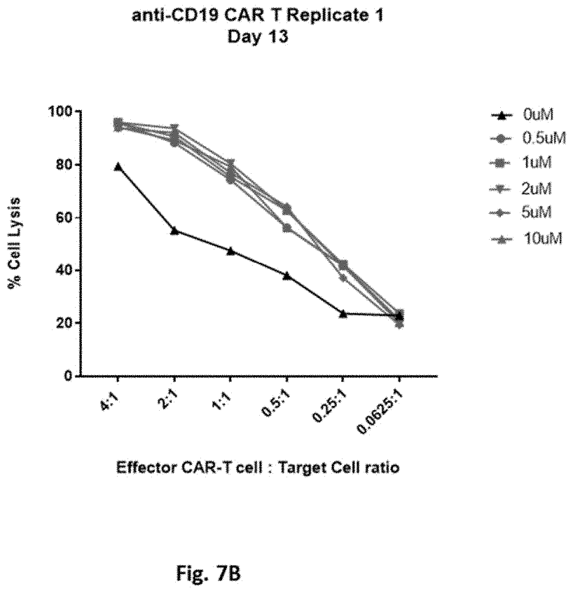

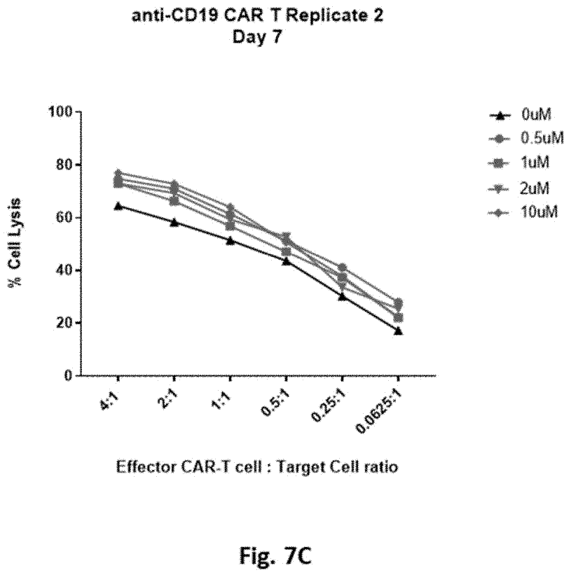

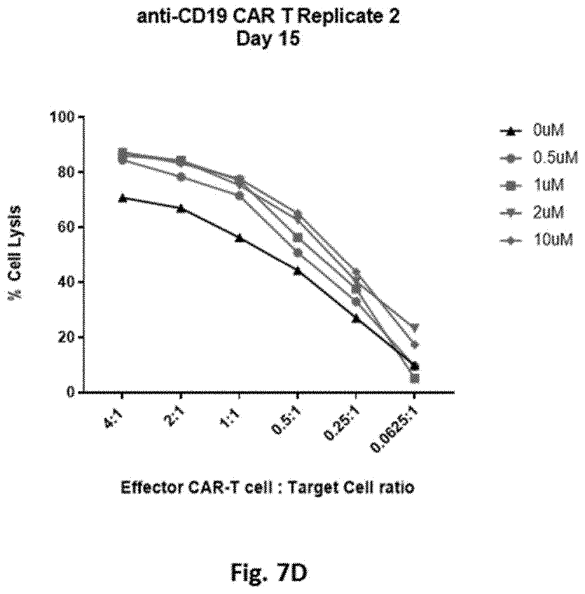

For anti-CD19 CAR-T cells, CD19+ Raji cells were labeled with eFluor670 and incubated with CAR-T cells at varying ratios. CAR-T cell cytotoxicity was analyzed at 24 hours by assessing labeled cells in the live gate compared to control sample. The results are shown in FIGS. 7A-7D. Compared with the CAR-T cells expanded without Lenalidomide, the Lenalidomide treated cells demonstrated comparable or higher killing capacities harvested on day 6/7 and day 13/15 in two independent studies, shown as higher percentage of cell lysis.

### Sequence Tables

The following tables provide details for the various nucleotide and amino acid sequences disclosed herein.

TABLE 1
sgRNA Sequences and Target Gene Sequences for TRAC, β2M, CD70, and Reg1

| | | | SEQ ID |
|---|---|---|---|
| | | | NO: |
| sgRNA Sequences | | | |
| TRAC sgRNA | Modified | A*G*A*GCAACAGUGCUGUGGCCguuuuagagcuag | 2 |
| (TA-1) | | aaauagcaaguuaaaauaaggcuaguccguuaucaa | |
| | | cuugaaaaaguggcaccgagucggugcU*U*U*U | |
| | Unmodified | AGAGCAACAGUGCUGUGGCCguuuuagagcuagaaa | 3 |
| | | uagcaaguuaaaauaaggcuaguccguuaucaacuu | |
| | | gaaaaaguggcaccgagucggugcUUUU | |
| TRAC sgRNA | Modified | A*G*A*GCAACAGUGCUGUGGCC | 4 |
| spacer | Unmodified | AGAGCAACAGUGCUGUGGCC | 5 |
| β2M sgRNA | Modified | G*C*U*ACUCUCUCUUUCUGGCCguuuuagagcuag | 6 |
| (β2M-1) | | aaauagcaaguuaaaauaaggcuaguccguuaucaa | |
| | | cuugaaaaaguggcaccgagucggugcU*U*U*U | |
| | Unmodified | GCUACUCUCUCUUUCUGGCCguuuuagagcuagaaa | 7 |
| | | uagcaaguuaaaauaaggcuaguccguuaucaacuu | |
| | | gaaaaaguggcaccgagucggugcUUUU | |
| β2M sgRNA | Modified | G*C*U*ACUCUCUCUUUCUGGCC | 8 |
| spacer | Unmodified | GCUACUCUCUCUUUCUGGCC | 9 |
| CD70 sgRNA | Modified | G*C*U*UUGGUCCCAUUGGUCGCguuuuagagcuag | 10 |
| (CD70-7) | | aaauagcaaguuaaaauaaggcuaguccguuaucaa | |
| | | cuugaaaaaguggcaccgagucggugcU*U*U*U | |
| | Unmodified | GCUUUGGUCCCAUUGGUCGCguuuuagagcuagaaa | 11 |
| | | uagcaaguuaaaauaaggcuaguccguuaucaacuu | |
| | | gaaaaaguggcaccgagucggugcUUUU | |
| CD70 sgRNA | Modified | G*C*U*UUGGUCCCAUUGGUCGC | 12 |
| spacer | Unmodified | GCUUUGGUCCCAUUGGUCGC | 13 |
| Reg1 sgRNA | Modified | mA*mC*mG*ACGCGUGGGUGGCAAGCguuuuagagc | 14 |
| (Z10) | | uagaaauagcaaguuaaaauaaggcuaguccguuau | |
| | | caacuugaaaaaguggcaccgagucggugcmU*mU* | |
| | | mU*U | |
| | Unmodified | ACGACGCGUGGGUGGCAAGCguuuuagagcuagaaa | 15 |
| | | uagcaaguuaaaauaaggcuaguccguuaucaacuu | |
| | | gaaaaaguggcaccgagucggugcmUUUU | |
| Reg1 sgRNA | Modified | mA*mC*mG*ACGCGUGGGUGGCAAGC | 16 |
| spacer | Unmodified | ACGACGCGTGGGTGGCAAGC | 17 |
| TGFBRII sgRNA | Modified | mU*mG*mC*GCAGGAUUUCUGGUUGUCACAGGguuu | 18 |
| EX4_T1 | | uagagcuagaaauagcaaguuaaaauaaggcuaguc | |
| | | cguuaucaacuugaaaaaguggcaccgagucggugc | |
| | | mU*mU*mU*U | |
| | Unmodified | UGCGCAGGAUUUCUGGUUGUCACAGGguuuuagagc | 19 |
| | | uagaaauagcaaguuaaaauaaggcuaguccguuau | |
| | | caacuugaaaaaguggcaccgagucggugcmUUUU | |
| TGFBRH sgRNA | Modified | mU*mG*mC*GCAGGAUUUCUGGUUGUCACAGG | 20 |
| EX4_T1 | | | |
| Spacer | Unmodified | UGCGCAGGAUUUCUGGUUGUCACAGG | 21 |
| TET2 sgRNA | Modified | C*A*U*UAGGACCUGCUCCUAGAguuuuagagcuag | 22 |
| Exon4_BG4 | | aaauagcaaguuaaaauaaggcuaguccguuaucaa | |
| | | cuugaaaaaguggcaccgagucggugcU*U*U*U | |
| | Unmodified | CAUUAGGACCUGCUCCUAGAguuuuagagcuagaaa | 23 |
| | | uagcaaguuaaaauaaggcuaguccguuaucaacuu | |
| | | gaaaaaguggcaccgagucggugcUUUU | |
| TET2 sgRNA | Modified | C*A*U*UAGGACCUGCUCCUAGA | 24 |
| Exon4_BG4 | | | |
| Spacer | Unmodified | CAUUAGGACCUGCUCCUAGA | 25 |

Target Sequences (PAM)

| | | |
|---|---|---|
| CD70 target | GCTTTGGTCCCATTGGTCGC (GGG) | 26 |
| sequence with | | |
| (PAM) | | |
| CD70 target | GCTTTGGTCCCATTGGTCGC | 27 |
| sequence | | |
| TRAC target | AGAGCAACAGTGCTGTGGCC (TGG) | 28 |
| sequence with | | |
| (PAM) | | |
| TRAC target | AGAGCAACAGTGCTGTGGCC | 29 |
| sequence | | |
| β2M target | | |
| sequence with | GCTACTCTCTCTTTCTGGCC (TGG) | 30 |
| (PAM) | | |
| β2M target | GCTACTCTCTCTTTCTGGCC | 31 |
| sequence | | |
| Reg1 target | ACGACGCGTGGGTGGCAAGC (GGG) | 32 |
| sequence with | | |
| (PAM) | | |
| Reg1 target | ACGACGCGTGGGTGGCAAGC | 33 |
| sequence | | |
| TGFBRII target | TGCGCAGGATTTCTGGTTGTCACAGG (AGG) | 34 |
| sequence with | | |
| (PAM) | | |
| TGFBRII target | TGCGCAGGATTTCTGGTTGTCACAGG | 35 |
| sequence | | |
| TET2 target | CATTAGGACCTGCTCCTAGA (TGG) | 36 |
| sequence with | | |
| (PAM) | | |
| TET2 target | CATTAGGACCTGCTCCTAGA | 44 |
| sequence | | |

Exemplary sgRNA Formulas

| | | |
|---|---|---|
| sgRNA sequence | nnnnnnnnnnnnnnnnnnnnguuuuagagcuagaaauagcaaguuaaaa | 45 |
| | uaaggcuaguccguuaucaacuugaaaaaguggcaccgagucggugcuu | |
| | uu | |
| sgRNA sequence | nnnnnnnnnnnnnnnnnnnnguuuuagagcuagaaauagcaaguuaaaa | 46 |
| | uaaggcuaguccguuaucaacuugaaaaaguggcaccgagucggugc | |
| sgRNA sequence | n (17-30) | 47 |
| | guuuuagagcuagaaauagcaaguuaaaauaaggcuaguccguua | |
| | ucaacuugaaaaaguggcaccgagucggugcu (1-8) | |

* indicates a nucleotide with a 2′-O-methyl phosphorothioate modification.
“n” refers to the spacer sequence at the 5 end.

TABLE 2
Edited TRAC Gene Sequence.

| | Sequence (Deletions indicated by dashes (-); | |
|---|---|---|
| Description | insertions indicated by bold) | SEQ ID NO: |
| TRAC gene | AA---------------------GAGCAACAAATCTGACT | 48 |
| edit | | |
| TRAC gene | AAGAGCAACAGTGCTGT-GCCTGGAGCAACAAATCTGACT | 49 |
| edit | | |
| TRAC gene | AAGAGCAACAGTG-------CTGGAGCAACAAATCTGACT | 50 |
| edit | | |
| TRAC gene | AAGAGCAACAGT------GCCTGGAGCAACAAATCTGACT | 51 |
| edit | | |
| TRAC gene | AAGAGCAACAGTG---------------------CTGACT | 52 |
| edit | | |
| TRAC gene | AAGAGCAACAGTGCTGTGGGCCTGGAGCAACAAATCTGACT | 53 |
| edit | | |
| TRAC gene | AAGAGCAACAGTGC--TGGCCTGGAGCAACAAATCTGACT | 55 |
| edit | | |
| TRAC gene | AAGAGCAACAGTGCTGTGTGCCTGGAGCAACAAATCTGACT | 56 |
| edit | | |

* These sequences would not present in the genetically modified T cells when a CAR-coding sequence is inserted into the disrupted TRAC gene in the products due to insertion of the CAR-coding sequence.

TABLE 3
Edited β2M Gene Sequence.

| | | SEQ |
|---|---|---|
| | Sequence (Deletions indicated by | ID |
| Description | dashes (-); insertions indicated by bold) | NO: |
| β2M gene-edit | CGTGGCCTTAGCTGTGCTCGCGCTACTCTCTCTTTCT- | 57 |
| | GCCTGGAGGCTATCCAGCGTGAGTCTCTCCTACCCTCCCGCT | |
| β2M gene-edit | CGTGGCCTTAGCTGTGCTCGCGCTACTCTCTCTTTC-- | 58 |
| | GCCTGGAGGCTATCCAGCGTGAGTCTCTCCTACCCTCCCGCT | |
| β2M gene-edit | CGTGGCCTTAGCTGTGCTCGCGCTACTCTCTCTTT----- | 59 |
| | CTGGAGGCTATCCAGCGTGAGTCTCTCCTACCCTCCCGCT | |
| β2M gene-edit | CGTGGCCTTAGCTGTGCTCGCGCTACTCTCTCTTTCTGGATA | 60 |
| | GCCTGGAGGCTATCCAGCGTGAGTCTCTCCTACCCTCCCGCT | |
| β2M gene-edit | CGTGGCCTTAGCTGTGCTCGC--------------------- | 61 |
| | ----GCTATCCAGCGTGAGTCTCTCCTACCCTCCCGCT | |
| β2M gene-edit | CGTGGCCTTAGCTGTGCTCGCGCTACTCTCTCTTTCTGTGGC | 62 |
| | CTGGAGGCTATCCAGCGTGAGTCTCTCCTACCCTCCCGCT | |

TABLE 4
Edited CD70 Gene Sequence.

| | Sequence (Deletions | SEQ |
|---|---|---|
| | indicated by dashes (-); | ID |
| Description | insertions indicated by bold) | NO: |
| CD70 | CACACCACGAGGCAGATCACCAAGCCCGCG-- | 63 |
| gene-edit | CAATGGGACCAAAGCAGCCCGCAGGACG | |
| CD70 | CACACCACGAGGCAGATCACCAAGCCCGCGAA | 64 |
| gene-edit | CCAATGGGACCAAAGCAGCCCGCAGGACG | |
| CD70 | CACACCACGAGGCAGATC------------ | 65 |
| gene-edit | ACCAATGGGACCAAAGCAGCCCGCAGGACG | |
| CD70 | CACACCACGAGGCAGATCACCAAGCCCGCG- | 66 |
| gene-edit | CCAATGGGACCAAAGCAGCCCGCAGGACG | |
| CD70 | CACACCACGAGGCAGATCACCAAGCCCGC- | 67 |
| gene-edit | ACCAATGGGACCAAAGCAGCCCGCAGGACG | |
| CD70 | CACACCACGAGGCAGATCACCA-------- | 68 |
| gene-edit | -----------------AGCCCGCAGGACG | |

TABLE 5
Edited Reg1 Gene Sequence.

| | Sequence (Deletions | SEQ |
|---|---|---|
| | indicated by dashes (-); | ID |
| Description | insertions indicated by bold) | NO: |
| Reg1 gene-edit | GTGGGTGGCAAAGCGGGTGGT | 69 |
| Reg1 gene-edit | GT-----------GGGTGGT | |
| Reg1 gene-edit | -----------GCGGGTGGT | |
| Reg1 gene-edit | GTGGGTGGC-AGCGGGTGGT | 70 |
| Reg1 gene-edit | ---------------GTGGT | |
| Reg1 gene-edit | GTG--------------GGT | |
| Reg1 gene-edit | ------------CGGGTGGT | |
| Reg1 gene-edit | -------------------- | |
| Reg1 gene-edit | GTGGGTGGC----------- | |
| Reg1 gene-edit | GTGGGTGGCATAGCGGGTGGT | 71 |
| Reg1 gene-edit | GTGGGTG------------- | |
| Reg1 gene-edit | GTGG---------------- | |
| Reg1 gene-edit | GTGGGTGG--AGCGGGTGGT | 72 |

TABLE 6
Edited TET2 Gene Sequence

| | Sequence (Deletions | SEQ |
|---|---|---|
| | indicated by dashes (-); | ID |
| Description | insertions indicated by bold) | NO |
| TET2 gene-edit | ACCTGCTCCTTAGATGGGTAT | 73 |
| TET2 gene-edit | ACCT---------------- | |
| TET2 gene-edit | ACCTGCTCC-AGATGGGTAT | 74 |
| TET2 gene-edit | ACCTGCT---AGATGGGTAT | 75 |
| TET2 gene-edit | ACCTG----------GGTAT | 76 |
| TET2 gene-edit | -------------------- | |
| TET2 gene-edit | ACCTGCTC-TAGATGGGTAT | 77 |
| TET2 gene-edit | A------------TGGGTAT | |
| TET2 gene-edit | ACCTG----TAGATGGGTAT | 78 |
| TET2 gene-edit | ACCTGCTCCTA--TGGGTAT | 79 |

TABLE 7
Chimeric Antigen Receptor Sequences

| SEQ | | |
|---|---|---|
| ID | | |
| NO | Description | Sequence |
| 80 | signal peptide | MLLLVTSLLLCELPHPAFLLIP |
| 81 | signal peptide | MALPVTALLLPLALLLHAARP |
| 82 | CD8a | TYIWAPLAGTCGVLLLSLVITLY |
| | transmembrane | |
| | domain | |
| 83 | 4- 1BB | AAACGGGGCAGAAAGAAACTCCTGTATATATTCAAACAACCATTTATGA |
| | nucleotide | GACCAGTACAAACTACTCAAGAGGAAGATGGCTGTAGCTGCCGATTTCC |
| | sequence | AGAAGAAGAAGAAGGAGGATGTGAACTG |
| 84 | 4-1BB amino | KRGRKKLLYIFKQPFMRPVQTTQEEDGCSCRFPEEEEGGCEL |
| | acid sequence | |
| 85 | CD28 nucleotide | TCAAAGCGGAGTAGGTTGTTGCATTCCGATTACATGAATATGACTCCTC |
| | sequence | GCCGGCCTGGGCCGACAAGAAAACATTACCAACCCTATGCCCCCCCACG |
| | | AGACTTCGCTGCGTACAGGTCC |
| 86 | CD28 amino | SKRSRLLHSDYMNMTPRRPGPTRKHYQPYAPPRDFAAYRS |
| | acid sequence | |
| 87 | CD3-zeta | CGAGTGAAGTTTTCCCGAAGCGCAGACGCTCCGGCATATCAGCAAGGAC |
| | nucleotide | AGAATCAGCTGTATAACGAACTGAATTTGGGACGCCGCGAGGAGTATGA |
| | sequence | CGTGCTTGATAAACGCCGGGGGAGAGACCCGGAAATGGGGGGTAAACCC |
| | | CGAAGAAAGAATCCCCAAGAAGGACTCTACAATGAACTCCAGAAGGATA |
| | | AGATGGCGGAGGCCTACTCAGAAATAGGTATGAAGGGCGAACGACGACG |
| | | GGGAAAAGGTCACGATGGCCTCTACCAAGGGTTGAGTACGGCAACCAAA |
| | | GATACGTACGATGCACTGCATATGCAGGCCCTGCCTCCCAGA |
| 88 | CD3-zeta amino | RVKFSRSADAPAYQQGQNQLYNELNLGRREEYDVLDKRRGRDPEMGGKP |
| | acid sequence | RRKNPQEGLYNELQKDKMAEAYSEIGMKGERRRGKGHDGLYQGLSTATK |
| | | DTYDALHMQALPPR |
| 89 | anti-CD19 VL | RASQDISKYLN |
| | CDR1 (Kabat) | |
| 90 | anti-CD19 VL | HTSRLHS |
| | CDR2 (Kabat) | |
| 91 | anti-CD19 VL | QQGNTLPYT |
| | CDR3 (Kabat) | |
| 92 | anti-CD19 VH | DYGVS |
| | CDR1 (Kabat) | |
| 93 | anti-CD19 VH | VIWGSETTYYNSALKS |
| | CDR2 (Kabat) | |
| 94 | anti-CD19 VH | HYYYGGSYAMDY |
| | CDR3 (Kabat) | |
| 95 | anti-CD19 VL | RASQDISKYLN |
| | CDR1 (Chothia) | |
| 96 | anti-CD19 VL | HTSRLHS |
| | CDR2 (Chothia) | |
| 97 | anti-CD19 VL | QQGNTLPYT |
| | CDR3 (Chothia) | |
| 98 | anti-CD19 VH | GVSLPDY |
| | CDR1 (Chothia) | |
| 99 | anti-CD19 VH | WGSET |
| | CDR2 (Chothia) | |
| 100 | anti-CD19 VH | HYYYGGSYAMDY |
| | CDR3 (Chothia) | |
| 101 | Anti-CD19 CAR | ATGCTTCTTTTGGTTACGTCTCTGTTGCTTTGCGAACTTCCTCATCCAG |
| | FMC63-28Z | CGTTCTTGCTGATCCCCGATATTCAGATGACTCAGACCACCAGTAGCTT |
| | (FMC63-CD8 | GTCTGCCTCACTGGGAGACCGAGTAACAATCTCCTGCAGGGCAAGTCAA |
| | [tm]-CD28[co- | GACATTAGCAAATACCTCAATTGGTACCAGCAGAAGCCCGACGGAACGG |
| | stimulatory | TAAAACTCCTCATCTATCATACGTCAAGGTTGCATTCCGGAGTACCGTC |
| | domain]-CD3z) | ACGATTTTCAGGTTCTGGGAGCGGAACTGACTATTCCTTGACTATTTCA |
| | | AACCTCGAGCAGGAGGACATTGCGACATATTTTTGTCAACAAGGTAATA |
| | | CCCTCCCTTACACTTTCGGAGGAGGAACCAAACTCGAAATTACCGGGTC |
| | | CACCAGTGGCTCTGGGAAGCCTGGCAGTGGAGAAGGTTCCACTAAAGGC |
| | | GAGGTGAAGCTCCAGGAGAGCGGCCCCGGTCTCGTTGCCCCCAGTCAAA |
| | | GCCTCTCTGTAACGTGCACAGTGAGTGGTGTATCATTGCCTGATTATGG |
| | | CGTCTCCTGGATAAGGCAGCCCCCGCGAAAGGGTCTTGAATGGCTTGGG |
| | | GTAATATGGGGCTCAGAGACAACGTATTATAACTCCGCTCTCAAAAGTC |
| | | GCTTGACGATAATAAAAGATAACTCCAAGAGTCAAGTTTTCCTTAAAAT |
| | | GAACAGTTTGCAGACTGACGATACCGCTATATATTATTGTGCTAAACAT |
| | | TATTACTACGGCGGTAGTTACGCGATGGATTATTGGGGGCAGGGGACTT |
| | | CTGTCACAGTCAGTAGTGCTGCTGCCTTTGTCCCGGTATTTCTCCCAGC |
| | | CAAACCGACCACGACTCCCGCCCCGCGCCCTCCGACACCCGCTCCCACC |
| | | ATCGCCTCTCAACCTCTTAGTCTTCGCCCCGAGGCATGCCGACCCGCCG |
| | | CCGGGGGTGCTGTTCATACGAGGGGCTTGGACTTCGCTTGTGATATTTA |
| | | CATTTGGGCTCCGTTGGCGGGTACGTGCGGCGTCCTTTTGTTGTCACTC |
| | | GTTATTACTTTGTATTGTAATCACAGGAATCGCTCAAAGCGGAGTAGGT |
| | | TGTTGCATTCCGATTACATGAATATGACTCCTCGCCGGCCTGGGCCGAC |
| | | AAGAAAACATTACCAACCCTATGCCCCCCCACGAGACTTCGCTGCGTAC |
| | | AGGTCCCGAGTGAAGTTTTCCCGAAGCGCAGACGCTCCGGCATATCAGC |
| | | AAGGACAGAATCAGCTGTATAACGAACTGAATTTGGGACGCCGCGAGGA |
| | | GTATGACGTGCTTGATAAACGCCGGGGGAGAGACCCGGAAATGGGGGGT |
| | | AAACCCCGAAGAAAGAATCCCCAAGAAGGACTCTACAATGAACTCCAGA |
| | | AGGATAAGATGGCGGAGGCCTACTCAGAAATAGGTATGAAGGGCGAACG |
| | | ACGACGGGGAAAAGGTCACGATGGCCTCTACCAAGGGTTGAGTACGGCA |
| | | ACCAAAGATACGTACGATGCACTGCATATGCAGGCCCTGCCTCCCAGA |
| 102 | Anti-CD19 CAR | DIQMTQTTSSLSASLGDRVTISCRASQDISKYLNWYQQKPDGTVKLLIY |
| | FMC63-28Z | HTSRLHSGVPSRFSGSGSGTDYSLTISNLEQEDIATYFCQQGNTLPYTF |
| | (FMC63-CD8[tm]- | GGGTKLEITGSTSGSGKPGSGEGSTKGEVKLQESGPGLVAPSQSLSVTC |
| | CD28[co- | TVSGVSLPDYGVSWIRQPPRKGLEWLGVIWGSETTYYNSALKSRLTI1K |
| | stimulatory | DNSKSQVFLKMNSLQTDDTAIYYCAKHYYYGGSYAMDYWGQGTSVTVSS |
| | domain]-CD3Z) | AAAFVPVFLPAKPTTTPAPRPPTPAPTIASQPLSLRPEACRPAAGGAVH |
| | Amino Acid | TRGLDFACDIYIWAPLAGTCGVLLLSLVITLYCNHRNRSKRSRLLHSDY |
| | No signal | MNMTPRRPGPTRKHYQPYAPPRDFAAYRSRVKFSRSADAPAYQQGQNQL |
| | peptide | YNELNLGRREEYDVLDKRRGRDPEMGGKPRRKNPQEGLYNELQKDKMAE |
| | | AYSEIGMKGERRRGKGHDGLYQGLSTATKDTYDALHMQALPPR |
| 156 | Anti-CD19 CAR | MLLLVTSLLLCELPHPAFLLIPDIQMTQTTSSLSASLGDRVTISCRASQ |
| | FMC63-28Z | DISKYLNWYQQKPDGTVKLLIYHTSRLHSGVPSRFSGSGSGTDYSLTIS |
| | (FMC63-CD8[tm]- | NLEQEDIATYFCQQGNTLPYTFGGGTKLEITGSTSGSGKPGSGEGSTKG |
| | CD28[co- | EVKLQESGPGLVAPSQSLSVTCTVSGVSLPDYGVSWIRQPPRKGLEWLG |
| | stimulatory | VIWGSETTYYNSALKSRLTIIKDNSKSQVFLKMNSLQTDDTAIYYCAKH |
| | domain]-CD3Z) | YYYGGSYAMDYWGQGTSVTVSSAAAFVPVFLPAKPTTTPAPRPPTPAPT |
| | Amino Acid | IASQPLSLRPEACRPAAGGAVHTRGLDFACDIYIWAPLAGTCGVLLLSL |
| | With signal | VITLYCNHRNRSKRSRLLHSDYMNMTPRRPGPTRKHYQPYAPPRDFAAY |
| | peptide | RSRVKFSRSADAPAYQQGQNQLYNELNLGRREEYDVLDKRRGRDPEMGG |
| | | KPRRKNPQEGLYNELQKDKMAEAYSEIGMKGERRRGKGHDGLYQGLSTA |
| | | TKDTYDALHMQALPPR |
| 103 | Anti-CD19 scFv | GATATTCAGATGACTCAGACCACCAGTAGCTTGTCTGCCTCACTGGGAG |
| | coding | ACCGAGTAACAATCTCCTGCAGGGCAAGTCAAGACATTAGCAAATACCT |
| | sequence | CAATTGGTACCAGCAGAAGCCCGACGGAACGGTAAAACTCCTCATCTAT |
| | | CATACGTCAAGGTTGCATTCCGGAGTACCGTCACGATTTTCAGGTTCTG |
| | | GGAGCGGAACTGACTATTCCTTGACTATTTCAAACCTCGAGCAGGAGGA |
| | | CATTGCGACATATTTTTGTCAACAAGGTAATACCCTCCCTTACACTTTC |
| | | GGAGGAGGAACCAAACTCGAAATTACCGGGTCCACCAGTGGCTCTGGGA |
| | | AGCCTGGCAGTGGAGAAGGTTCCACTAAAGGCGAGGTGAAGCTCCAGGA |
| | | GAGCGGCCCCGGTCTCGTTGCCCCCAGTCAAAGCCTCTCTGTAACGTGC |
| | | ACAGTGAGTGGTGTATCATTGCCTGATTATGGCGTCTCCTGGATAAGGC |
| | | AGCCCCCGCGAAAGGGTCTTGAATGGCTTGGGGTAATATGGGGCTCAGA |
| | | GACAACGTATTATAACTCCGCTCTCAAAAGTCGCTTGACGATAATAAAA |
| | | GATAACTCCAAGAGTCAAGTTTTCCTTAAAATGAACAGTTTGCAGACTG |
| | | ACGATACCGCTATATATTATTGTGCTAAACATTATTACTACGGCGGTAG |
| | | TTACGCGATGGATTATTGGGGGCAGGGGACTTCTGTCACAGTCAGTAGT |
| 104 | CD19 scFv amino | DIQMTQTTSSLSASLGDRVTISCRASQDISKYLNWYQQKPDGTVKLLIY |
| | acid sequence | HTSRLHSGVPSRFSGSGSGTDYSLTISNLEQEDIATYFCQQGNTLPYTF |
| | Linker | GGGTKLEITGSTSGSGKPGSGEGSTKGEVKLQESGPGLVAPSQSLSVTC |
| | underlined | TVSGVSLPDYGVSWIRQPPRKGLEWLGVIWGSETTYYNSALKSRLTIIK |
| | | DNSKSQVFLKMNSLQTDDTAIYYCAKHYYYGGSYAMDYWGQGTSVTVSS |
| 105 | CD8a | GCTGCTGCCTTTGTCCCGGTATTTCTCCCAGCCAAACCGACCACGACTC |
| | extracellular + | CCGCCCCGCGCCCTCCGACACCCGCTCCCACCATCGCCTCTCAACCTCT |
| | CD8a | TAGTCTTCGCCCCGAGGCATGCCGACCCGCCGCCGGGGGTGCTGTTCAT |
| | transmembrane + | ACGAGGGGCTTGGACTTCGCTTGTGATATTTACATTTGGGCTCCGTTGG |
| | 5′ Linker | CGGGTACGTGCGGCGTCCTTTTGTTGTCACTCGTTATTACTTTGTATTG |
| | (underlined) | TAATCACAGGAATCGC |
| 106 | CD8a | TTTGTCCCGGTATTTCTCCCAGCCAAACCGACCACGACTCCCGCCCCGC |
| | extracellular + | GCCCTCCGACACCCGCTCCCACCATCGCCTCTCAACCTCTTAGTCTTCG |
| | CD8a | CCCCGAGGCATGCCGACCCGCCGCCGGGGGTGCTGTTCATACGAGGGGC |
| | transmembrane | TTGGACTTCGCTTGTGATATTTACATTTGGGCTCCGTTGGCGGGTACGT |
| | (without linker) | GCGGCGTCCTTTTGTTGTCACTCGTTATTACTTTGTATTGTAATCACAG |
| | | GAATCGC |
| 107 | CD8a | FVPVFLPAKPTTTPAPRPPTPAPTIASQPLSLRPEACRPAAGGAVHTRG |
| | extracellular + | |
| | CD8a | LDFACD IYIWAPLAGTCGVLLLSLVITLYCNHRNR |
| | transmembrane | |
| 108 | CD19 VH | EVKLQESGPGLVAPSQSLSVTCTVSGVSLPDYGVSWIRQPPRKGLEWLG |
| | | VIWGSETTYYNSALKSRLTIIKDNSKSQVFLKMNSLQTDDTAIYYCAKH |
| | | YYYGGSYAMDYWGQGTSVTVSS |
| 109 | CD19 VL | DIQMTQTTSSLSASLGDRVTISCRASQDISKYLNWYQQKPDGTVKLLIY |
| | | HTSRLHSGVPSRFSGSGSGTDYSLTISNLEQEDIATYFCQQGNTLPYTF |
| | | GGGTKLEIT |
| 110 | CD19 linker | GSTSGSGKPGSGEGSTKG |
| 111 | CD70 VL CDR1 | RASKSVSTSGYSFMH |
| | (Kabat) | |
| 112 | CD70 VL CDR1 | SKSVSTSGYSF |
| | (Chothia) | |
| 113 | CD70 VL CDR2 | LASNLES |
| | (Kabat) | |
| N/A | CD70 VL CDR2 | LAS |
| | (Chothia) | |
| 114 | CD70 VL CDR3 | QHSREVPWT |
| | (Kabat) | |
| 115 | CD70 VL CDR3 | SREVPW |
| | (Chothia) | |
| 116 | CD70 VH CDR1 | NY GMN |
| | (Kabat) | |
| 117 | CD70 VH CDR1 | GYTF TNYGMN |
| | (Chothia) | |
| 118 | CD70 VH CDR2 | WINTYTGEPTYADAFKG |
| | (Kabat) | |
| 119 | CD70 VH CDR2 | NTYTGE |
| | (Chothia) | |
| 120 | CD70 VH CDR3 | DYGDYGMDY |
| | (Kabat) | |
| 121 | CD70 VH CDR3 | CARD YGDYGMDYWG |
| | (Chothia) | |
| 122 | CD70 CAR | ATGGCGCTTCCGGTGACAGCACTGCTCCTCCCCTTGGCGCTGTTGCTCC |
| | nucleotide | ACGCAGCAAGGCCGCAGGTCCAGTTGGTGCAAAGCGGGGCGGAGGTGAA |
| | sequence | AAAACCCGGCGCTTCCGTGAAGGTGTCCTGTAAGGCGTCCGGTTATACG |
| | (CD70B scFV | TTCACGAACTACGGGATGAATTGGGTTCGCCAAGCGCCGGGGCAGGGAC |
| | with 41BB) | TGAAATGGATGGGGTGGATAAATACCTACACCGGCGAACCTACATACGC |
| | | CGACGCTTTTAAAGGGCGAGTCACTATGACGCGCGATACCAGCATATCC |
| | | ACCGCATACATGGAGCTGTCCCGACTCCGGTCAGACGACACGGCTGTCT |
| | | ACTATTGTGCTCGGGACTATGGCGATTATGGCATGGACTACTGGGGTCA |
| | | GGGTACGACTGTAACAGTTAGTAGTGGTGGAGGCGGCAGTGGCGGGGGG |
| | | GGAAGCGGAGGAGGGGGTTCTGGTGACATAGTTATGACCCAATCCCCAG |
| | | ATAGTTTGGCGGTTTCTCTGGGCGAGAGGGCAACGATTAATTGTCGCGC |
| | | ATCAAAGAGCGTTTCAACGAGCGGATATTCTTTTATGCATTGGTACCAG |
| | | CAAAAACCCGGACAACCGCCGAAGCTGCTGATCTACTTGGCTTCAAATC |
| | | TTGAGTCTGGGGTGCCGGACCGATTTTCTGGTAGTGGAAGCGGAACTGA |
| | | CTTTACGCTCACGATCAGTTCACTGCAGGCTGAGGATGTAGCGGTCTAT |
| | | TATTGCCAGCACAGTAGAGAAGTCCCCTGGACCTTCGGTCAAGGCACGA |
| | | AAGTAGAAATTAAAAGTGCTGCTGCCTTTGTCCCGGTATTTCTCCCAGC |
| | | CAAACCGACCACGACTCCCGCCCCGCGCCCTCCGACACCCGCTCCCACC |
| | | ATCGCCTCTCAACCTCTTAGTCTTCGCCCCGAGGCATGCCGACCCGCCG |
| | | CCGGGGGTGCTGTTCATACGAGGGGCTTGGACTTCGCTTGTGATATTTA |
| | | CATTTGGGCTCCGTTGGCGGGTACGTGCGGCGTCCTTTTGTTGTCACTC |
| | | GTTATTACTTTGTATTGTAATCACAGGAATCGCAAACGGGGCAGAAAGA |
| | | AACTCCTGTATATATTCAAACAACCATTTATGAGACCAGTACAAACTAC |
| | | TCAAGAGGAAGATGGCTGTAGCTGCCGATTTCCAGAAGAAGAAGAAGGA |
| | | GGATGTGAACTGCGAGTGAAGTTTTCCCGAAGCGCAGACGCTCCGGCAT |
| | | ATCAGCAAGGACAGAATCAGCTGTATAACGAACTGAATTTGGGACGCCG |
| | | CGAGGAGTATGACGTGCTTGATAAACGCCGGGGGAGAGACCCGGAAATG |
| | | GGGGGTAAACCCCGAAGAAAGAATCCCCAAGAAGGACTCTACAATGAAC |
| | | TCCAGAAGGATAAGATGGCGGAGGCCTACTCAGAAATAGGTATGAAGGG |
| | | CGAACGACGACGGGGAAAAGGTCACGATGGCCTCTACCAAGGGTTGAGT |
| | | ACGGCAACCAAAGATACGTACGATGCACTGCATATGCAGGCCCTGCCTC |
| | | CCAGATAA |
| 123 | CD70 CAR amino | QVQLVQSGAEVKKPGASVKVSCKASGYTFTNYGMNWVRQAPGQGLKWMG |
| | acid sequence | WINTYTGEPTYADAFKGRVTMTRDTSISTAYMELSRLRSDDTAVYYCAR |
| | (CD70B scFv | DYGDYGMDYWGQGTTVTVSSGGGGSGGGGSGGGGSGDIVMTQSPDSLAV |
| | with 41BB) | SLGERATINCRASKSVSTSGYSFMHWYQQKPGQPPKLLIYLASNLESGV |
| | No signal | PDRFSGSGSGTDFTLTISSLQAEDVAVYYCQHSREVPWTFGQGTKVEIK |
| | peptide | SAAAFVPVFLPAKPTTTPAPRPPTPAPTIASQPLSLRPEACRPAAGGAV |
| | | HTRGLDFACDIYIWAPLAGTCGVLLLSLVITLYCNHRNRKRGRKKLLYI |
| | | FKQPFMRPVQTTQEEDGCSCRFPEEEEGGCELRVKFSRSADAPAYQQGQ |
| | | NQLYNELNLGRREEYDVLDKRRGRDPEMGGKPRRKNPQEGLYNELQKDK |
| | | MAEAYSEIGMKGERRRGKGHDGLYQGLSTATKDTYDALHMQALPPR |
| 157 | CD70 CAR amino | MALPVTALLLPLALLLHAARPQVQLVQSGAEVKKPGASVKVSCKASGYT |
| | acid sequence | FTNYGMNWVRQAPGQGLKWMGWINTYTGEPTYADAFKGRVTMTRDTSIS |
| | (CD70B scFv | TAYMELSRLRSDDTAVYYCARDYGDYGMDYWGQGTTVTVSSGGGGSGGG |
| | with 41BB) | GSGGGGSGDIVMTQSPDSLAVSLGERATINCRASKSVSTSGYSFMHWYQ |
| | With signal | QKPGQPPKLLIYLASNLESGVPDRFSGSGSGTDFTLTISSLQAEDVAVY |
| | peptide | YCQHSREVPWTFGQGTKVEIKSAAAFVPVFLPAKPTTTPAPRPPTPAPT |
| | | IASQPLSLRPEACRPAAGGAVHTRGLDFACDIYIWAPLAGTCGVLLLSL |
| | | VITLYCNHRNRKRGRKKLLYIFKQPFMRPVQTTQEEDGCSCRFPEEEEG |
| | | GCELRVKFSRSADAPAYQQGQNQLYNELNLGRREEYDVLDKRRGRDPEM |
| | | GGKPRRKNPQEGLYNELQKDKMAEAYSEIGMKGERRRGKGHDGLYQGLS |
| | | TATKDTYDALHMQALPPR |
| 124 | CD70A | GATATAGTTATGACCCAATCACCCGATAGTCTTGCGGTAAGCCTGGGGG |
| | scFv nucleotide | AGCGAGCAACAATAAACTGTCGGGCATCAAAATCCGTCAGTACAAGCGG |
| | sequence | GTATTCATTCATGCACTGGTATCAACAGAAACCCGGTCAGCCACCCAAG |
| | | CTCCTGATTTATCTTGCGTCTAATCTTGAGTCCGGCGTCCCAGACCGGT |
| | | TTTCCGGCTCCGGGAGCGGCACGGATTTTACTCTTACTATTTCTAGCCT |
| | | TCAGGCCGAAGATGTGGCGGTATACTACTGCCAGCATTCAAGGGAAGTT |
| | | CCTTGGACGTTCGGTCAGGGCACGAAAGTGGAAATTAAAGGCGGGGGGG |
| | | GATCCGGCGGGGGAGGGTCTGGAGGAGGTGGCAGTGGTCAGGTCCAACT |
| | | GGTGCAGTCCGGGGCAGAGGTAAAAAAACCCGGCGCGTCTGTTAAGGTT |
| | | TCATGCAAGGCCAGTGGATATACTTTCACCAATTACGGAATGAACTGGG |
| | | TGAGGCAGGCCCCTGGTCAAGGCCTGAAATGGATGGGATGGATAAACAC |
| | | GTACACCGGTGAACCTACCTATGCCGATGCCTTTAAGGGTCGGGTTACG |
| | | ATGACGAGAGACACCTCCATATCAACAGCCTACATGGAGCTCAGCAGAT |
| | | TGAGGAGTGACGATACGGCAGTCTATTACTGTGCAAGAGACTACGGCGA |
| | | TTATGGCATGGATTACTGGGGCCAGGGCACTACAGTAACCGTTTCCAGC |
| 125 | CD70A | DIVMTQSPDSLAVSLGERATINCRASKSVSTSGYSFMHWYQQKPGQPPK |
| | scFv amino | LLIYLASNLESGVPDRFSGSGSGTDFTLTISSLQAEDVAVYYCQHSREV |
| | acid sequence | PWTFGQGTKVEIKGGGGSGGGGSGGGGSGQVQLVQSGAEVKKPGASVKV |
| | (linker | SCKASGYTFTNYGMNWVRQAPGQGLKWMGWINTYTGEPTYADAFKGRVT |
| | underlined) | MTRDTSISTAYMELSRLRSDDTAVYYCARDYGDYGMDYWGQGTTVTVSS |
| 126 | CD70B | CAGGTCCAGTTGGTGCAAAGCGGGGCGGAGGTGAAAAAACCCGGCGCTT |
| | scFv nucleotide | CCGTGAAGGTGTCCTGTAAGGCGTCCGGTTATACGTTCACGAACTACGG |
| | sequence | GATGAATTGGGTTCGCCAAGCGCCGGGGCAGGGACTGAAATGGATGGGG |
| | | TGGATAAATACCTACACCGGCGAACCTACATACGCCGACGCTTTTAAAG |
| | | GGCGAGTCACTATGACGCGCGATACCAGCATATCCACCGCATACATGGA |
| | | GCTGTCCCGACTCCGGTCAGACGACACGGCTGTCTACTATTGTGCTCGG |
| | | GACTATGGCGATTATGGCATGGACTACTGGGGTCAGGGTACGACTGTAA |
| | | CAGTTAGTAGTGGTGGAGGCGGCAGTGGCGGGGGGGGAAGCGGAGGAGG |
| | | GGGTTCTGGTGACATAGTTATGACCCAATCCCCAGATAGTTTGGCGGTT |
| | | TCTCTGGGCGAGAGGGCAACGATTAATTGTCGCGCATCAAAGAGCGTTT |
| | | CAACGAGCGGATATTCTTTTATGCATTGGTACCAGCAAAAACCCGGACA |
| | | ACCGCCGAAGCTGCTGATCTACTTGGCTTCAAATCTTGAGTCTGGGGTG |
| | | CCGGACCGATTTTCTGGTAGTGGAAGCGGAACTGACTTTACGCTCACGA |
| | | TCAGTTCACTGCAGGCTGAGGATGTAGCGGTCTATTATTGCCAGCACAG |
| | | TAGAGAAGTCCCCTGGACCTTCGGTCAAGGCACGAAAGTAGAAATTAAA |
| 127 | CD70B | QVQLVQSGAEVKKPGASVKVSCKASGYTFTNYGMNWVRQAPGQGLKWMG |
| | scFv amino | WINTYTGEPTYADAFKGRVTMTRDTSISTAYMELSRLRSDDTAVYYCAR |
| | acid sequence | DYGDYGMDYWGQGTTVTVSSGGGGSGGGGSGGGGSGDIVMTQSPDSLAV |
| | (linker | SLGERATINCRASKSVSTSGYSFMHWYQQKPGQPPKLLIYLASNLESGV |
| | underlined) | PDRFSGSGSGTDFTLTISSLQAEDVAVYYCQHSREVPWTFGQGTKVEIK |
| 128 | CD70 VH | QVQLVQSGAEVKKPGASVKVSCKASGYTFTNYGMNWVRQAPGQGLKWMG |
| | | WINTYTGEPTYADAFKGRVTMTRDTSISTAYMELSRLRSDDTAVYYCAR |
| | | DYGDYGMDYWGQGTTVTVSS |
| 129 | CD70 VL | DIVMTQSPDSLAVSLGERATINCRASKSVSTSGYSFMHWYQQKPGQPPK |
| | | LLIYLASNLESGVPDRFSGSGSGTDFTLTISSLQAEDVAVYYCQHSREV |
| | | PWTFGQGTKVEIK |
| 130 | BCMA CAR | ATGGCGCTTCCGGTGACAGCACTGCTCCTCCCCTTGGCGCTGTTGCTCC |
| | nucleotide | ACGCAGCAAGGCCGCAGGTGCAGCTGGTGCAGAGCGGAGCCGAGCTCAA |
| | sequence | GAAGCCCGGAGCCTCCGTGAAGGTGAGCTGCAAGGCCAGCGGCAACACC |
| | | CTGACCAACTACGTGATCCACTGGGTGAGACAAGCCCCCGGCCAAAGGC |
| | | TGGAGTGGATGGGCTACATCCTGCCCTACAACGACCTGACCAAGTACAG |
| | | CCAGAAGTTCCAGGGCAGGGTGACCATCACCAGGGATAAGAGCGCCTCC |
| | | ACCGCCTATATGGAGCTGAGCAGCCTGAGGAGCGAGGACACCGCTGTGT |
| | | ACTACTGTACAAGGTGGGACTGGGACGGCTTCTTTGACCCCTGGGGCCA |
| | | GGGCACAACAGTGACCGTCAGCAGCGGCGGCGGAGGCAGCGGCGGCGGC |
| | | GGCAGCGGCGGAGGCGGAAGCGAAATCGTGATGACCCAGAGCCCCGCCA |
| | | CACTGAGCGTGAGCCCTGGCGAGAGGGCCAGCATCTCCTGCAGGGCTAG |
| | | CCAAAGCCTGGTGCACAGCAACGGCAACACCCACCTGCACTGGTACCAG |
| | | CAGAGACCCGGACAGGCTCCCAGGCTGCTGATCTACAGCGTGAGCAACA |
| | | GGTTCTCCGAGGTGCCTGCCAGGTTTAGCGGCAGCGGAAGCGGCACCGA |
| | | CTTTACCCTGACCATCAGCAGCGTGGAGTCCGAGGACTTCGCCGTGTAT |
| | | TACTGCAGCCAGACCAGCCACATCCCTTACACCTTCGGCGGCGGCACCA |
| | | AGCTGGAGATCAAAAGTGCTGCTGCCTTTGTCCCGGTATTTCTCCCAGC |
| | | CAAACCGACCACGACTCCCGCCCCGCGCCCTCCGACACCCGCTCCCACC |
| | | ATCGCCTCTCAACCTCTTAGTCTTCGCCCCGAGGCATGCCGACCCGCCG |
| | | CCGGGGGTGCTGTTCATACGAGGGGCTTGGACTTCGCTTGTGATATTTA |
| | | CATTTGGGCTCCGTTGGCGGGTACGTGCGGCGTCCTTTTGTTGTCACTC |
| | | GTTATTACTTTGTATTGTAATCACAGGAATCGCAAACGGGGCAGAAAGA |
| | | AACTCCTGTATATATTCAAACAACCATTTATGAGACCAGTACAAACTAC |
| | | TCAAGAGGAAGATGGCTGTAGCTGCCGATTTCCAGAAGAAGAAGAAGGA |
| | | GGATGTGAACTGCGAGTGAAGTTTTCCCGAAGCGCAGACGCTCCGGCAT |
| | | ATCAGCAAGGACAGAATCAGCTGTATAACGAACTGAATTTGGGACGCCG |
| | | CGAGGAGTATGACGTGCTTGATAAACGCCGGGGGAGAGACCCGGAAATG |
| | | GGGGGTAAACCCCGAAGAAAGAATCCCCAAGAAGGACTCTACAATGAAC |
| | | TCCAGAAGGATAAGATGGCGGAGGCCTACTCAGAAATAGGTATGAAGGG |
| | | CGAACGACGACGGGGAAAAGGTCACGATGGCCTCTACCAAGGGTTGAGT |
| | | ACGGCAACCAAAGATACGTACGATGCACTGCATATGCAGGCCCTGCCTC |
| | | CCAGA |
| 131 | BCMA CAR amino | QVQLVQSGAELKKPGASVKVSCKASGNTLTNYVIHWVRQAPGQRLEWMG |
| | acid sequence | YILPYNDLTKYSQKFQGRVTITRDKSASTAYMELSSLRSEDTAVYYCTR |
| | No signal | WDWDGFFDPWGQGTTVTVSSGGGGSGGGGSGGGGSEIVMTQSPATLSVS |
| | peptide | PGERASISCRASQSLVHSNGNTHLHWYQQRPGQAPRLLIYSVSNRFSEV |
| | | SAAAFVPVFLPAKPTTTPAPRPPTPAPTIASQPLSLRPEACRPAAGGAV |
| | | HTRGLDFACDIYIWAPLAGTCGVLLLSLVITLYCNHRNRKRGRKKLLYI |
| | | FKQPFMRPVQTTQEEDGCSCRFPEEEEGGCELRVKFSRSADAPAYQQGQ |
| | | NQLYNELNLGRREEYDVLDKRRGRDPEMGGKPRRKNPQEGLYNELQKDK |
| | | MAEAYSEIGMKGERRRGKGHDGLYQGLSTATKDTYDALHMQALPPR |
| 158 | BCMA CAR amino | MALPVTALLLPLALLLHAARPQVQLVQSGAELKKPGASVKVSCKASGNT |
| | acid sequence | LTNYVIHWVRQAPGQRLEWMGYILPYNDLTKYSQKFQGRVTITRDKSAS |
| | With signal | TAYMELSSLRSEDTAVYYCTRWDWDGEEDPWGQGTTVTVSSGGGGSGGG |
| | peptide | GSGGGGSEIVMTQSPATLSVSPGERASISCRASQSLVHSNGNTHLHWYQ |
| | | QRPGQAPRLLIYSVSNRFSEVPARFSGSGSGTDFTLTISSVESEDFAVY |
| | | YCSQTSHIPYTFGGGTKLEIKSAAAFVPVFLPAKPTTTPAPRPPTPAPT |
| | | IASQPLSLRPEACRPAAGGAVHTRGLDFACDIYIWAPLAGTCGVLLLSL |
| | | VITLYCNHRNRKRGRKKLLYIFKQPFMRPVQTTQEEDGCSCRFPEEEEG |
| | | GCELRVKFSRSADAPAYQQGQNQLYNELNLGRREEYDVLDKRRGRDPEM |
| | | GGKPRRKNPQEGLYNELQKDKMAEAYSEIGMKGERRRGKGHDGLYQGLS |
| | | TATKDTYDALHMQALPPR |
| 132 | BCMA | CAGGTGCAGCTGGTGCAGAGCGGAGCCGAGCTCAAGAAGCCCGGAGCCT |
| | scFv nucleotide | CCGTGAAGGTGAGCTGCAAGGCCAGCGGCAACACCCTGACCAACTACGT |
| | sequence | GATCCACTGGGTGAGACAAGCCCCCGGCCAAAGGCTGGAGTGGATGGGC |
| | | TACATCCTGCCCTACAACGACCTGACCAAGTACAGCCAGAAGTTCCAGG |
| | | GCAGGGTGACCATCACCAGGGATAAGAGCGCCTCCACCGCCTATATGGA |
| | | GCTGAGCAGCCTGAGGAGCGAGGACACCGCTGTGTACTACTGTACAAGG |
| | | TGGGACTGGGACGGCTTCTTTGACCCCTGGGGCCAGGGCACAACAGTGA |
| | | CCGTCAGCAGCGGCGGCGGAGGCAGCGGCGGCGGCGGCAGCGGCGGAGG |
| | | CGGAAGCGAAATCGTGATGACCCAGAGCCCCGCCACACTGAGCGTGAGC |
| | | CCTGGCGAGAGGGCCAGCATCTCCTGCAGGGCTAGCCAAAGCCTGGTGC |
| | | ACAGCAACGGCAACACCCACCTGCACTGGTACCAGCAGAGACCCGGACA |
| | | GGCTCCCAGGCTGCTGATCTACAGCGTGAGCAACAGGTTCTCCGAGGTG |
| | | CCTGCCAGGTTTAGCGGCAGCGGAAGCGGCACCGACTTTACCCTGACCA |
| | | TCAGCAGCGTGGAGTCCGAGGACTTCGCCGTGTATTACTGCAGCCAGAC |
| | | CAGCCACATCCCTTACACCTTCGGCGGCGGCACCAAGCTGGAGATCAAA |
| 133 | BCMA | QVQLVQSGAELKKPGASVKVSCKASGNTLTNYVIHWVRQAPGQRLEWMG |
| | scFv amino acid | YILPYNDLTKYSQKFQGRVTITRDKSASTAYMELSSLRSEDTAVYYCTR |
| | sequence | WDWDGFFDPWGQGTTVTVSSGGGGSGGGGSGGGGSEIVMTQSPATLSVS |
| | (linker | PGERASISCRASQSLVHSNGNTHLHWYQQRPGQAPRLLIYSVSNRFSEV |
| | underlined) | PARFSGSGSGTDFTLTISSVESEDFAVYYCSQTSHIPYTFGGGTKLEIK |
| 134 | BCMA VH | QVQLVQSGAELKKPGASVKVSCKASGNTLTNYVIHWVRQAPGQRLEWMG |
| | | YILPYNDLTKYSQKFQGRVTITRDKSASTAYMELSSLRSEDTAVYYCTR |
| | | WDWDGFFDPWGQGTTVTVSS |
| 135 | BCMA VL | EIVMTQSPATLSVSPGERASISCRASQSLVHSNGNTHLHWYQQRPGQAP |
| | | RLLIYSVSNRFSEVPARFSGSGSGTDFTLTISSVESEDFAVYYCSQTSH |
| | | IPYTFGGGTKLEIK |
| 136 | BCMA VL CDR1 | RASQSLVHSNGNTHLH |
| | (Kabat & | |
| | Chothia) | |
| 137 | BCMA VL CDR2 | SVSNRFS |
| | (Kabat & | |
| | Chothia) | |
| 138 | BCMA VL CDR3 | SQTSHIPYT |
| | (Kabat) | |
| 139 | BCMA VL CDR3 | SQTSHIPYT |
| | (Chothia) | |
| 140 | BCMA VH CDR1 | NYVIH |
| | (Kabat) | |
| 141 | BCMA VH CDR1 | GNTLTNY |
| | (Chothia) | |
| 142 | BCMA VH CDR2 | YILPYNDLTKYSQKFQG |
| | (Kabat) | |
| 143 | BCMA VH CDR2 | LPYNDL |
| | (Chothia) | |
| 144 | BCMA VH CDR3 | WDWDGFFDP |
| | (Kabat) | |
| 145 | BCMA VH CDR3 | WDWDGFFDP |
| | (Chothia) | |

TABLE 8
AAV Donor Template Sequences

| | | |
|---|---|---|
| 146 | Left ITR (5′ ITR) | TTGGCCACTCCCTCTCTGCGCGCTCGCTCGCTCACTGAGGCCGGGCGAC |
| | | CAAAGGTCGCCCGACGCCCGGGCTTTGCCCGGGCGGCCTCAGTGAGCGA |
| | | GCGAGCGCGCAGAGAGGGAGTGGCCAACTCCATCACTAGGGGTTCCT |
| 147 | Left ITR (5′ ITR) | CCTGCAGGCAGCTGCGCGCTCGCTCGCTCACTGAGGCCGCCCGGGCGTC |
| | (alternate) | GGGCGACCTTTGGTCGCCCGGCCTCAGTGAGCGAGCGAGCGCGCAGAGA |
| | | GGGAGTGGCCAACTCCATCACTAGGGGTTCCT |
| 148 | Right ITR (3′ ITR) | AGGAACCCCTAGTGATGGAGTTGGCCACTCCCTCTCTGCGCGCTCGCTC |
| | | GCTCACTGAGGCCGCCCGGGCAAAGCCCGGGCGTCGGGCGACCTTTGGT |
| | | CGCCCGGCCTCAGTGAGCGAGCGAGCGCGCAGAGAGGGAGTGGCCAA |
| 149 | Right ITR (3′ ITR) | AGGAACCCCTAGTGATGGAGTTGGCCACTCCCTCTCTGCGCGCTCGCTC |
| | (alternate) | GCTCACTGAGGCCGGGCGACCAAAGGTCGCCCGACGCCCGGGCTTTGCC |
| | | CGGGCGGCCTCAGTGAGCGAGCGAGCGCGCAGCTGCCTGCAGG |
| 150 | TRAC-LHA | GAGATGTAAGGAGCTGCTGTGACTTGCTCAAGGCCTTATATCGAGTAAA |
| | (800b p) | CGGTAGTGCTGGGGCTTAGACGCAGGTGTTCTGATTTATAGTTCAAAAC |
| | | CTCTATCAATGAGAGAGCAATCTCCTGGTAATGTGATAGATTTCCCAAC |
| | | TTAATGCCAACATACCATAAACCTCCCATTCTGCTAATGCCCAGCCTAA |
| | | GTTGGGGAGACCACTCCAGATTCCAAGATGTACAGTTTGCTTTGCTGGG |
| | | CCTTTTTCCCATGCCTGCCTTTACTCTGCCAGAGTTATATTGCTGGGGT |
| | | TTTGAAGAAGATCCTATTAAATAAAAGAATAAGCAGTATTATTAAGTAG |
| | | CCCTGCATTTCAGGTTTCCTTGAGTGGCAGGCCAGGCCTGGCCGTGAAC |
| | | GTTCACTGAAATCATGGCCTCTTGGCCAAGATTGATAGCTTGTGCCTGT |
| | | CCCTGAGTCCCAGTCCATCACGAGCAGCTGGTTTCTAAGATGCTATTTC |
| | | CCGTATAAAGCATGAGACCGTGACTTGCCAGCCCCACAGAGCCCCGCCC |
| | | TTGTCCATCACTGGCATCTGGACTCCAGCCTGGGTTGGGGCAAAGAGGG |
| | | AAATGAGATCATGTCCTAACCCTGATCCTCTTGTCCCACAGATATCCAG |
| | | AACCCTGACCCTGCCGTGTACCAGCTGAGAGACTCTAAATCCAGTGACA |
| | | AGTCTGTCTGCCTATTCACCGATTTTGATTCTCAAACAAATGTGTCACA |
| | | AAGTAAGGATTCTGATGTGTATATCACAGACAAAACTGTGCTAGACATG |
| | | AGGTCTATGGACTTCA |
| 151 | TRAC-RHA | TGGAGCAACAAATCTGACTTTGCATGTGCAAACGCCTTCAACAACAGCA |
| | (800b p) | TTATTCCAGAAGACACCTTCTTCCCCAGCCCAGGTAAGGGCAGCTTTGG |
| | | TGCCTTCGCAGGCTGTTTCCTTGCTTCAGGAATGGCCAGGTTCTGCCCA |
| | | GAGCTCTGGTCAATGATGTCTAAAACTCCTCTGATTGGTGGTCTCGGCC |
| | | TTATCCATTGCCACCAAAACCCTCTTTTTACTAAGAAACAGTGAGCCTT |
| | | GTTCTGGCAGTCCAGAGAATGACACGGGAAAAAAGCAGATGAAGAGAAG |
| | | GTGGCAGGAGAGGGCACGTGGCCCAGCCTCAGTCTCTCCAACTGAGTTC |
| | | CTGCCTGCCTGCCTTTGCTCAGACTGTTTGCCCCTTACTGCTCTTCTAG |
| | | GCCTCATTCTAAGCCCCTTCTCCAAGTTGCCTCTCCTTATTTCTCCCTG |
| | | TCTGCCAAAAAATCTTTCCCAGCTCACTAAGTCAGTCTCACGCAGTCAC |
| | | TCATTAACCCACCAATCACTGATTGTGCCGGCACATGAATGCACCAGGT |
| | | GTTGAAGTGGAGGAATTAAAAAGTCAGATGAGGGGTGTGCCCAGAGGAA |
| | | GCACCATTCTAGTTGGGGGAGCCCATCTGTCAGCTGGGAAAAGTCCAAA |
| | | TAACTTCAGATTGGAATGTGTTTTAACTCAGGGTTGAGAAAACAGCTAC |
| | | CTTCAGGACAAAAGTCAGGGAAGGGCTCTCTGAAGAAATGCTACTTGAA |
| | | GATACCAGCCCTACCAAGGGCAGGGAGAGGACCCTATAGAGGCCTGGGA |
| | | CAGGAGCTCAATGAGAAAGG |
| 152 | EF1a | GGCTCCGGTGCCCGTCAGTGGGCAGAGCGCACATCGCCCACAGTCCCCG |
| | | AGAAGTTGGGGGGAGGGGTCGGCAATTGAACCGGTGCCTAGAGAAGGTG |
| | | GCGCGGGGTAAACTGGGAAAGTGATGTCGTGTACTGGCTCCGCCTTTTT |
| | | CCCGAGGGTGGGGGAGAACCGTATATAAGTGCAGTAGTCGCCGTGAACG |
| | | TTCTTTTTCGCAACGGGTTTGCCGCCAGAACACAGGTAAGTGCCGTGTG |
| | | TGGTTCCCGCGGGCCTGGCCTCTTTACGGGTTATGGCCCTTGCGTGCCT |
| | | TGAATTACTTCCACTGGCTGCAGTACGTGATTCTTGATCCCGAGCTTCG |
| | | GGTTGGAAGTGGGTGGGAGAGTTCGAGGCCTTGCGCTTAAGGAGCCCCT |
| | | TCGCCTCGTGCTTGAGTTGAGGCCTGGCCTGGGCGCTGGGGCCGCCGCG |
| | | TGCGAATCTGGTGGCACCTTCGCGCCTGTCTCGCTGCTTTCGATAAGTC |
| | | TCTAGCCATTTAAAATTTTTGATGACCTGCTGCGACGCTTTTTTTCTGG |
| | | CAAGATAGTCTTGTAAATGCGGGCCAAGATCTGCACACTGGTATTTCGG |
| | | TTTTTGGGGCCGCGGGCGGCGACGGGGCCCGTGCGTCCCAGCGCACATG |
| | | TTCGGCGAGGCGGGGCCTGCGAGCGCGGCCACCGAGAATCGGACGGGGG |
| | | TAGTCTCAAGCTGGCCGGCCTGCTCTGGTGCCTGGCCTCGCGCCGCCGT |
| | | GTATCGCCCCGCCCTGGGCGGCAAGGCTGGCCCGGTCGGCACCAGTTGC |
| | | GTGAGCGGAAAGATGGCCGCTTCCCGGCCCTGCTGCAGGGAGCTCAAAA |
| | | TGGAGGACGCGGCGCTCGGGAGAGCGGGCGGGTGAGTCACCCACACAAA |
| | | GGAAAAGGGCCTTTCCGTCCTCAGCCGTCGCTTCATGTGACTCCACGGA |
| | | GTACCGGGCGCCGTCCAGGCACCTCGATTAGTTCTCGAGCTTTTGGAGT |
| | | ACGTCGTCTTTAGGTTGGGGGGAGGGGTTTTATGCGATGGAGTTTCCCC |
| | | ACACTGAGTGGGTGGAGACTGAAGTTAGGCCAGCTTGGCACTTGATGTA |
| | | ATTCTCCTTGGAATTTGCCCTTTTTGAGTTTGGATCTTGGTTCATTCTC |
| | | AAGCCTCAGACAGTGGTTCAAAGTTTTTTTCTTCCATTTCAGGTGTCGT |
| | | GA |
| 153 | CD19 | GAGATGTAAGGAGCTGCTGTGACTTGCTCAAGGCCTTATATCGAGTAAA |
| | LHA to RHA | CGGTAGTGCTGGGGCTTAGACGCAGGTGTTCTGATTTATAGTTCAAAAC |
| | | CTCTATCAATGAGAGAGCAATCTCCTGGTAATGTGATAGATTTCCCAAC |
| | | TTAATGCCAACATACCATAAACCTCCCATTCTGCTAATGCCCAGCCTAA |
| | | GTTGGGGAGACCACTCCAGATTCCAAGATGTACAGTTTGCTTTGCTGGG |
| | | CCTTTTTCCCATGCCTGCCTTTACTCTGCCAGAGTTATATTGCTGGGGT |
| | | TTTGAAGAAGATCCTATTAAATAAAAGAATAAGCAGTATTATTAAGTAG |
| | | CCCTGCATTTCAGGTTTCCTTGAGTGGCAGGCCAGGCCTGGCCGTGAAC |
| | | GTTCACTGAAATCATGGCCTCTTGGCCAAGATTGATAGCTTGTGCCTGT |
| | | CCCTGAGTCCCAGTCCATCACGAGCAGCTGGTTTCTAAGATGCTATTTC |
| | | CCGTATAAAGCATGAGACCGTGACTTGCCAGCCCCACAGAGCCCCGCCC |
| | | TTGTCCATCACTGGCATCTGGACTCCAGCCTGGGTTGGGGCAAAGAGGG |
| | | AAATGAGATCATGTCCTAACCCTGATCCTCTTGTCCCACAGATATCCAG |
| | | AACCCTGACCCTGCCGTGTACCAGCTGAGAGACTCTAAATCCAGTGACA |
| | | AGTCTGTCTGCCTATTCACCGATTTTGATTCTCAAACAAATGTGTCACA |
| | | AAGTAAGGATTCTGATGTGTATATCACAGACAAAACTGTGCTAGACATG |
| | | AGGTCTATGGACTTCAGGCTCCGGTGCCCGTCAGTGGGCAGAGCGCACA |
| | | TCGCCCACAGTCCCCGAGAAGTTGGGGGGAGGGGTCGGCAATTGAACCG |
| | | GTGCCTAGAGAAGGTGGCGCGGGGTAAACTGGGAAAGTGATGTCGTGTA |
| | | CTGGCTCCGCCTTTTTCCCGAGGGTGGGGGAGAACCGTATATAAGTGCA |
| | | GTAGTCGCCGTGAACGTTCTTTTTCGCAACGGGTTTGCCGCCAGAACAC |
| | | AGGTAAGTGCCGTGTGTGGTTCCCGCGGGCCTGGCCTCTTTACGGGTTA |
| | | TGGCCCTTGCGTGCCTTGAATTACTTCCACTGGCTGCAGTACGTGATTC |
| | | TTGATCCCGAGCTTCGGGTTGGAAGTGGGTGGGAGAGTTCGAGGCCTTG |
| | | CGCTTAAGGAGCCCCTTCGCCTCGTGCTTGAGTTGAGGCCTGGCCTGGG |
| | | CGCTGGGGCCGCCGCGTGCGAATCTGGTGGCACCTTCGCGCCTGTCTCG |
| | | CTGCTTTCGATAAGTCTCTAGCCATTTAAAATTTTTGATGACCTGCTGC |
| | | GACGCTTTTTTTCTGGCAAGATAGTCTTGTAAATGCGGGCCAAGATCTG |
| | | CACACTGGTATTTCGGTTTTTGGGGCCGCGGGCGGCGACGGGGCCCGTG |
| | | CGTCCCAGCGCACATGTTCGGCGAGGCGGGGCCTGCGAGCGCGGCCACC |
| | | GAGAATCGGACGGGGGTAGTCTCAAGCTGGCCGGCCTGCTCTGGTGCCT |
| | | GGCCTCGCGCCGCCGTGTATCGCCCCGCCCTGGGCGGCAAGGCTGGCCC |
| | | GGTCGGCACCAGTTGCGTGAGCGGAAAGATGGCCGCTTCCCGGCCCTGC |
| | | TGCAGGGAGCTCAAAATGGAGGACGCGGCGCTCGGGAGAGCGGGCGGGT |
| | | GAGTCACCCACACAAAGGAAAAGGGCCTTTCCGTCCTCAGCCGTCGCTT |
| | | CATGTGACTCCACGGAGTACCGGGCGCCGTCCAGGCACCTCGATTAGTT |
| | | CTCGAGCTTTTGGAGTACGTCGTCTTTAGGTTGGGGGGAGGGGTTTTAT |
| | | GCGATGGAGTTTCCCCACACTGAGTGGGTGGAGACTGAAGTTAGGCCAG |
| | | CTTGGCACTTGATGTAATTCTCCTTGGAATTTGCCCTTTTTGAGTTTGG |
| | | ATCTTGGTTCATTCTCAAGCCTCAGACAGTGGTTCAAAGTTTTTTTCTT |
| | | CCATTTCAGGTGTCGTGACCACCATGCTTCTTTTGGTTACGTCTCTGTT |
| | | GCTTTGCGAACTTCCTCATCCAGCGTTCTTGCTGATCCCCGATATTCAG |
| | | ATGACTCAGACCACCAGTAGCTTGTCTGCCTCACTGGGAGACCGAGTAA |
| | | CAATCTCCTGCAGGGCAAGTCAAGACATTAGCAAATACCTCAATTGGTA |
| | | CCAGCAGAAGCCCGACGGAACGGTAAAACTCCTCATCTATCATACGTCA |
| | | AGGTTGCATTCCGGAGTACCGTCACGATTTTCAGGTTCTGGGAGCGGAA |
| | | CTGACTATTCCTTGACTATTTCAAACCTCGAGCAGGAGGACATTGCGAC |
| | | ATATTTTTGTCAACAAGGTAATACCCTCCCTTACACTTTCGGAGGAGGA |
| | | ACCAAACTCGAAATTACCGGGTCCACCAGTGGCTCTGGGAAGCCTGGCA |
| | | GTGGAGAAGGTTCCACTAAAGGCGAGGTGAAGCTCCAGGAGAGCGGCCC |
| | | CGGTCTCGTTGCCCCCAGTCAAAGCCTCTCTGTAACGTGCACAGTGAGT |
| | | GGTGTATCATTGCCTGATTATGGCGTCTCCTGGATAAGGCAGCCCCCGC |
| | | GAAAGGGTCTTGAATGGCTTGGGGTAATATGGGGCTCAGAGACAACGTA |
| | | TTATAACTCCGCTCTCAAAAGTCGCTTGACGATAATAAAAGATAACTCC |
| | | AAGAGTCAAGTTTTCCTTAAAATGAACAGTTTGCAGACTGACGATACCG |
| | | CTATATATTATTGTGCTAAACATTATTACTACGGCGGTAGTTACGCGAT |
| | | GGATTATTGGGGGCAGGGGACTTCTGTCACAGTCAGTAGTGCTGCTGCC |
| | | TTTGTCCCGGTATTTCTCCCAGCCAAACCGACCACGACTCCCGCCCCGC |
| | | GCCCTCCGACACCCGCTCCCACCATCGCCTCTCAACCTCTTAGTCTTCG |
| | | CCCCGAGGCATGCCGACCCGCCGCCGGGGGTGCTGTTCATACGAGGGGC |
| | | TTGGACTTCGCTTGTGATATTTACATTTGGGCTCCGTTGGCGGGTACGT |
| | | GCGGCGTCCTTTTGTTGTCACTCGTTATTACTTTGTATTGTAATCACAG |
| | | GAATCGCTCAAAGCGGAGTAGGTTGTTGCATTCCGATTACATGAATATG |
| | | ACTCCTCGCCGGCCTGGGCCGACAAGAAAACATTACCAACCCTATGCCC |
| | | CCCCACGAGACTTCGCTGCGTACAGGTCCCGAGTGAAGTTTTCCCGAAG |
| | | CGCAGACGCTCCGGCATATCAGCAAGGACAGAATCAGCTGTATAACGAA |
| | | CTGAATTTGGGACGCCGCGAGGAGTATGACGTGCTTGATAAACGCCGGG |
| | | GGAGAGACCCGGAAATGGGGGGTAAACCCCGAAGAAAGAATCCCCAAGA |
| | | AGGACTCTACAATGAACTCCAGAAGGATAAGATGGCGGAGGCCTACTCA |
| | | GAAATAGGTATGAAGGGCGAACGACGACGGGGAAAAGGTCACGATGGCC |
| | | TCTACCAAGGGTTGAGTACGGCAACCAAAGATACGTACGATGCACTGCA |
| | | TATGCAGGCCCTGCCTCCCAGATAATAATAAAATCGCTATCCATCGAAG |
| | | ATGGATGTGTGTTGGTTTTTTGTGTGTGGAGCAACAAATCTGACTTTGC |
| | | ATGTGCAAACGCCTTCAACAACAGCATTATTCCAGAAGACACCTTCTTC |
| | | CCCAGCCCAGGTAAGGGCAGCTTTGGTGCCTTCGCAGGCTGTTTCCTTG |
| | | CTTCAGGAATGGCCAGGTTCTGCCCAGAGCTCTGGTCAATGATGTCTAA |
| | | AACTCCTCTGATTGGTGGTCTCGGCCTTATCCATTGCCACCAAAACCCT |
| | | CTTTTTACTAAGAAACAGTGAGCCTTGTTCTGGCAGTCCAGAGAATGAC |
| | | ACGGGAAAAAAGCAGATGAAGAGAAGGTGGCAGGAGAGGGCACGTGGCC |
| | | CAGCCTCAGTCTCTCCAACTGAGTTCCTGCCTGCCTGCCTTTGCTCAGA |
| | | CTGTTTGCCCCTTACTGCTCTTCTAGGCCTCATTCTAAGCCCCTTCTCC |
| | | AAGTTGCCTCTCCTTATTTCTCCCTGTCTGCCAAAAAATCTTTCCCAGC |
| | | TCACTAAGTCAGTCTCACGCAGTCACTCATTAACCCACCAATCACTGAT |
| | | TGTGCCGGCACATGAATGCACCAGGTGTTGAAGTGGAGGAATTAAAAAG |
| | | TCAGATGAGGGGTGTGCCCAGAGGAAGCACCATTCTAGTTGGGGGAGCC |
| | | CATCTGTCAGCTGGGAAAAGTCCAAATAACTTCAGATTGGAATGTGTTT |
| | | TAACTCAGGGTTGAGAAAACAGCTACCTTCAGGACAAAAGTCAGGGAAG |
| | | GGCTCTCTGAAGAAATGCTACTTGAAGATACCAGCCCTACCAAGGGCAG |
| | | GGAGAGGACCCTATAGAGGCCTGGGACAGGAGCTCAATGAGAAAGG |
| 154 | CD70 | GAGATGTAAGGAGCTGCTGTGACTTGCTCAAGGCCTTATATCGAGTAAA |
| | LHA to RHA | CGGTAGTGCTGGGGCTTAGACGCAGGTGTTCTGATTTATAGTTCAAAAC |
| | (CD7OB scFV with | CTCTATCAATGAGAGAGCAATCTCCTGGTAATGTGATAGATTTCCCAAC |
| | 41BB) | TTAATGCCAACATACCATAAACCTCCCATTCTGCTAATGCCCAGCCTAA |
| | | GTTGGGGAGACCACTCCAGATTCCAAGATGTACAGTTTGCTTTGCTGGG |
| | | CCTTTTTCCCATGCCTGCCTTTACTCTGCCAGAGTTATATTGCTGGGGT |
| | | TTTGAAGAAGATCCTATTAAATAAAAGAATAAGCAGTATTATTAAGTAG |
| | | CCCTGCATTTCAGGTTTCCTTGAGTGGCAGGCCAGGCCTGGCCGTGAAC |
| | | GTTCACTGAAATCATGGCCTCTTGGCCAAGATTGATAGCTTGTGCCTGT |
| | | CCCTGAGTCCCAGTCCATCACGAGCAGCTGGTTTCTAAGATGCTATTTC |
| | | CCGTATAAAGCATGAGACCGTGACTTGCCAGCCCCACAGAGCCCCGCCC |
| | | TTGTCCATCACTGGCATCTGGACTCCAGCCTGGGTTGGGGCAAAGAGGG |
| | | AAATGAGATCATGTCCTAACCCTGATCCTCTTGTCCCACAGATATCCAG |
| | | AACCCTGACCCTGCCGTGTACCAGCTGAGAGACTCTAAATCCAGTGACA |
| | | AGTCTGTCTGCCTATTCACCGATTTTGATTCTCAAACAAATGTGTCACA |
| | | AAGTAAGGATTCTGATGTGTATATCACAGACAAAACTGTGCTAGACATG |
| | | AGGTCTATGGACTTCAGGCTCCGGTGCCCGTCAGTGGGCAGAGCGCACA |
| | | TCGCCCACAGTCCCCGAGAAGTTGGGGGGAGGGGTCGGCAATTGAACCG |
| | | GTGCCTAGAGAAGGTGGCGCGGGGTAAACTGGGAAAGTGATGTCGTGTA |
| | | CTGGCTCCGCCTTTTTCCCGAGGGTGGGGGAGAACCGTATATAAGTGCA |
| | | GTAGTCGCCGTGAACGTTCTTTTTCGCAACGGGTTTGCCGCCAGAACAC |
| | | AGGTAAGTGCCGTGTGTGGTTCCCGCGGGCCTGGCCTCTTTACGGGTTA |
| | | TGGCCCTTGCGTGCCTTGAATTACTTCCACTGGCTGCAGTACGTGATTC |
| | | TTGATCCCGAGCTTCGGGTTGGAAGTGGGTGGGAGAGTTCGAGGCCTTG |
| | | CGCTTAAGGAGCCCCTTCGCCTCGTGCTTGAGTTGAGGCCTGGCCTGGG |
| | | CGCTGGGGCCGCCGCGTGCGAATCTGGTGGCACCTTCGCGCCTGTCTCG |
| | | CTGCTTTCGATAAGTCTCTAGCCATTTAAAATTTTTGATGACCTGCTGC |
| | | GACGCTTTTTTTCTGGCAAGATAGTCTTGTAAATGCGGGCCAAGATCTG |
| | | CACACTGGTATTTCGGTTTTTGGGGCCGCGGGCGGCGACGGGGCCCGTG |
| | | CGTCCCAGCGCACATGTTCGGCGAGGCGGGGCCTGCGAGCGCGGCCACC |
| | | GAGAATCGGACGGGGGTAGTCTCAAGCTGGCCGGCCTGCTCTGGTGCCT |
| | | GGCCTCGCGCCGCCGTGTATCGCCCCGCCCTGGGCGGCAAGGCTGGCCC |
| | | GGTCGGCACCAGTTGCGTGAGCGGAAAGATGGCCGCTTCCCGGCCCTGC |
| | | TGCAGGGAGCTCAAAATGGAGGACGCGGCGCTCGGGAGAGCGGGCGGGT |
| | | GAGTCACCCACACAAAGGAAAAGGGCCTTTCCGTCCTCAGCCGTCGCTT |
| | | CATGTGACTCCACGGAGTACCGGGCGCCGTCCAGGCACCTCGATTAGTT |
| | | CTCGAGCTTTTGGAGTACGTCGTCTTTAGGTTGGGGGGAGGGGTTTTAT |
| | | GCGATGGAGTTTCCCCACACTGAGTGGGTGGAGACTGAAGTTAGGCCAG |
| | | CTTGGCACTTGATGTAATTCTCCTTGGAATTTGCCCTTTTTGAGTTTGG |
| | | ATCTTGGTTCATTCTCAAGCCTCAGACAGTGGTTCAAAGTTTTTTTCTT |
| | | CCATTTCAGGTGTCGTGACCACCATGGCGCTTCCGGTGACAGCACTGCT |
| | | CCTCCCCTTGGCGCTGTTGCTCCACGCAGCAAGGCCGCAGGTCCAGTTG |
| | | GTGCAAAGCGGGGCGGAGGTGAAAAAACCCGGCGCTTCCGTGAAGGTGT |
| | | CCTGTAAGGCGTCCGGTTATACGTTCACGAACTACGGGATGAATTGGGT |
| | | TCGCCAAGCGCCGGGGCAGGGACTGAAATGGATGGGGTGGATAAATACC |
| | | TACACCGGCGAACCTACATACGCCGACGCTTTTAAAGGGCGAGTCACTA |
| | | TGACGCGCGATACCAGCATATCCACCGCATACATGGAGCTGTCCCGACT |
| | | CCGGTCAGACGACACGGCTGTCTACTATTGTGCTCGGGACTATGGCGAT |
| | | TATGGCATGGACTACTGGGGTCAGGGTACGACTGTAACAGTTAGTAGTG |
| | | GTGGAGGCGGCAGTGGCGGGGGGGGAAGCGGAGGAGGGGGTTCTGGTGA |
| | | CATAGTTATGACCCAATCCCCAGATAGTTTGGCGGTTTCTCTGGGCGAG |
| | | AGGGCAACGATTAATTGTCGCGCATCAAAGAGCGTTTCAACGAGCGGAT |
| | | ATTCTTTTATGCATTGGTACCAGCAAAAACCCGGACAACCGCCGAAGCT |
| | | GCTGATCTACTTGGCTTCAAATCTTGAGTCTGGGGTGCCGGACCGATTT |
| | | TCTGGTAGTGGAAGCGGAACTGACTTTACGCTCACGATCAGTTCACTGC |
| | | AGGCTGAGGATGTAGCGGTCTATTATTGCCAGCACAGTAGAGAAGTCCC |
| | | CTGGACCTTCGGTCAAGGCACGAAAGTAGAAATTAAAAGTGCTGCTGCC |
| | | TTTGTCCCGGTATTTCTCCCAGCCAAACCGACCACGACTCCCGCCCCGC |
| | | GCCCTCCGACACCCGCTCCCACCATCGCCTCTCAACCTCTTAGTCTTCG |
| | | CCCCGAGGCATGCCGACCCGCCGCCGGGGGTGCTGTTCATACGAGGGGC |
| | | TTGGACTTCGCTTGTGATATTTACATTTGGGCTCCGTTGGCGGGTACGT |
| | | GCGGCGTCCTTTTGTTGTCACTCGTTATTACTTTGTATTGTAATCACAG |
| | | GAATCGCAAACGGGGCAGAAAGAAACTCCTGTATATATTCAAACAACCA |
| | | TTTATGAGACCAGTACAAACTACTCAAGAGGAAGATGGCTGTAGCTGCC |
| | | GATTTCCAGAAGAAGAAGAAGGAGGATGTGAACTGCGAGTGAAGTTTTC |
| | | CCGAAGCGCAGACGCTCCGGCATATCAGCAAGGACAGAATCAGCTGTAT |
| | | AACGAACTGAATTTGGGACGCCGCGAGGAGTATGACGTGCTTGATAAAC |
| | | GCCGGGGGAGAGACCCGGAAATGGGGGGTAAACCCCGAAGAAAGAATCC |
| | | CCAAGAAGGACTCTACAATGAACTCCAGAAGGATAAGATGGCGGAGGCC |
| | | TACTCAGAAATAGGTATGAAGGGCGAACGACGACGGGGAAAAGGTCACG |
| | | ATGGCCTCTACCAAGGGTTGAGTACGGCAACCAAAGATACGTACGATGC |
| | | ACTGCATATGCAGGCCCTGCCTCCCAGATAATAATAAAATCGCTATCCA |
| | | TCGAAGATGGATGTGTGTTGGTTTTTTGTGTGTGGAGCAACAAATCTGA |
| | | CTTTGCATGTGCAAACGCCTTCAACAACAGCATTATTCCAGAAGACACC |
| | | TTCTTCCCCAGCCCAGGTAAGGGCAGCTTTGGTGCCTTCGCAGGCTGTT |
| | | TCCTTGCTTCAGGAATGGCCAGGTTCTGCCCAGAGCTCTGGTCAATGAT |
| | | GTCTAAAACTCCTCTGATTGGTGGTCTCGGCCTTATCCATTGCCACCAA |
| | | AACCCTCTTTTTACTAAGAAACAGTGAGCCTTGTTCTGGCAGTCCAGAG |
| | | AATGACACGGGAAAAAAGCAGATGAAGAGAAGGTGGCAGGAGAGGGCAC |
| | | GTGGCCCAGCCTCAGTCTCTCCAACTGAGTTCCTGCCTGCCTGCCTTTG |
| | | CTCAGACTGTTTGCCCCTTACTGCTCTTCTAGGCCTCATTCTAAGCCCC |
| | | TTCTCCAAGTTGCCTCTCCTTATTTCTCCCTGTCTGCCAAAAAATCTTT |
| | | CCCAGCTCACTAAGTCAGTCTCACGCAGTCACTCATTAACCCACCAATC |
| | | ACTGATTGTGCCGGCACATGAATGCACCAGGTGTTGAAGTGGAGGAATT |
| | | AAAAAGTCAGATGAGGGGTGTGCCCAGAGGAAGCACCATTCTAGTTGGG |
| | | GGAGCCCATCTGTCAGCTGGGAAAAGTCCAAATAACTTCAGATTGGAAT |
| | | GTGTTTTAACTCAGGGTTGAGAAAACAGCTACCTTCAGGACAAAAGTCA |
| | | GGGAAGGGCTCTCTGAAGAAATGCTACTTGAAGATACCAGCCCTACCAA |
| | | GGGCAGGGAGAGGACCCTATAGAGGCCTGGGACAGGAGCTCAATGAGAA |
| | | AGG |
| 155 | BCMA | GAGATGTAAGGAGCTGCTGTGACTTGCTCAAGGCCTTATATCGAGTAAA |
| | LHA to RHA | CGGTAGTGCTGGGGCTTAGACGCAGGTGTTCTGATTTATAGTTCAAAAC |
| | | CTCTATCAATGAGAGAGCAATCTCCTGGTAATGTGATAGATTTCCCAAC |
| | | TTAATGCCAACATACCATAAACCTCCCATTCTGCTAATGCCCAGCCTAA |
| | | GTTGGGGAGACCACTCCAGATTCCAAGATGTACAGTTTGCTTTGCTGGG |
| | | CCTTTTTCCCATGCCTGCCTTTACTCTGCCAGAGTTATATTGCTGGGGT |
| | | TTTGAAGAAGATCCTATTAAATAAAAGAATAAGCAGTATTATTAAGTAG |
| | | CCCTGCATTTCAGGTTTCCTTGAGTGGCAGGCCAGGCCTGGCCGTGAAC |
| | | GTTCACTGAAATCATGGCCTCTTGGCCAAGATTGATAGCTTGTGCCTGT |
| | | CCCTGAGTCCCAGTCCATCACGAGCAGCTGGTTTCTAAGATGCTATTTC |
| | | CCGTATAAAGCATGAGACCGTGACTTGCCAGCCCCACAGAGCCCCGCCC |
| | | TTGTCCATCACTGGCATCTGGACTCCAGCCTGGGTTGGGGCAAAGAGGG |
| | | AAATGAGATCATGTCCTAACCCTGATCCTCTTGTCCCACAGATATCCAG |
| | | AACCCTGACCCTGCCGTGTACCAGCTGAGAGACTCTAAATCCAGTGACA |
| | | AGTCTGTCTGCCTATTCACCGATTTTGATTCTCAAACAAATGTGTCACA |
| | | AAGTAAGGATTCTGATGTGTATATCACAGACAAAACTGTGCTAGACATG |
| | | AGGTCTATGGACTTCAGGCTCCGGTGCCCGTCAGTGGGCAGAGCGCACA |
| | | TCGCCCACAGTCCCCGAGAAGTTGGGGGGAGGGGTCGGCAATTGAACCG |
| | | GTGCCTAGAGAAGGTGGCGCGGGGTAAACTGGGAAAGTGATGTCGTGTA |
| | | CTGGCTCCGCCTTTTTCCCGAGGGTGGGGGAGAACCGTATATAAGTGCA |
| | | GTAGTCGCCGTGAACGTTCTTTTTCGCAACGGGTTTGCCGCCAGAACAC |
| | | AGGTAAGTGCCGTGTGTGGTTCCCGCGGGCCTGGCCTCTTTACGGGTTA |
| | | TGGCCCTTGCGTGCCTTGAATTACTTCCACTGGCTGCAGTACGTGATTC |
| | | TTGATCCCGAGCTTCGGGTTGGAAGTGGGTGGGAGAGTTCGAGGCCTTG |
| | | CGCTTAAGGAGCCCCTTCGCCTCGTGCTTGAGTTGAGGCCTGGCCTGGG |
| | | CGCTGGGGCCGCCGCGTGCGAATCTGGTGGCACCTTCGCGCCTGTCTCG |
| | | CTGCTTTCGATAAGTCTCTAGCCATTTAAAATTTTTGATGACCTGCTGC |
| | | GACGCTTTTTTTCTGGCAAGATAGTCTTGTAAATGCGGGCCAAGATCTG |
| | | CACACTGGTATTTCGGTTTTTGGGGCCGCGGGCGGCGACGGGGCCCGTG |
| | | CGTCCCAGCGCACATGTTCGGCGAGGCGGGGCCTGCGAGCGCGGCCACC |
| | | GAGAATCGGACGGGGGTAGTCTCAAGCTGGCCGGCCTGCTCTGGTGCCT |
| | | GGCCTCGCGCCGCCGTGTATCGCCCCGCCCTGGGCGGCAAGGCTGGCCC |
| | | GGTCGGCACCAGTTGCGTGAGCGGAAAGATGGCCGCTTCCCGGCCCTGC |
| | | TGCAGGGAGCTCAAAATGGAGGACGCGGCGCTCGGGAGAGCGGGCGGGT |
| | | GAGTCACCCACACAAAGGAAAAGGGCCTTTCCGTCCTCAGCCGTCGCTT |
| | | CATGTGACTCCACGGAGTACCGGGCGCCGTCCAGGCACCTCGATTAGTT |
| | | CTCGAGCTTTTGGAGTACGTCGTCTTTAGGTTGGGGGGAGGGGTTTTAT |
| | | GCGATGGAGTTTCCCCACACTGAGTGGGTGGAGACTGAAGTTAGGCCAG |
| | | CTTGGCACTTGATGTAATTCTCCTTGGAATTTGCCCTTTTTGAGTTTGG |
| | | ATCTTGGTTCATTCTCAAGCCTCAGACAGTGGTTCAAAGTTTTTTTCTT |
| | | CCATTTCAGGTGTCGTGACCACCATGGCGCTTCCGGTGACAGCACTGCT |
| | | CCTCCCCTTGGCGCTGTTGCTCCACGCAGCAAGGCCGCAGGTGCAGCTG |
| | | GTGCAGAGCGGAGCCGAGCTCAAGAAGCCCGGAGCCTCCGTGAAGGTGA |
| | | GCTGCAAGGCCAGCGGCAACACCCTGACCAACTACGTGATCCACTGGGT |
| | | GAGACAAGCCCCCGGCCAAAGGCTGGAGTGGATGGGCTACATCCTGCCC |
| | | TACAACGACCTGACCAAGTACAGCCAGAAGTTCCAGGGCAGGGTGACCA |
| | | TCACCAGGGATAAGAGCGCCTCCACCGCCTATATGGAGCTGAGCAGCCT |
| | | GAGGAGCGAGGACACCGCTGTGTACTACTGTACAAGGTGGGACTGGGAC |
| | | GGCTTCTTTGACCCCTGGGGCCAGGGCACAACAGTGACCGTCAGCAGCG |
| | | GCGGCGGAGGCAGCGGCGGCGGCGGCAGCGGCGGAGGCGGAAGCGAAAT |
| | | CGTGATGACCCAGAGCCCCGCCACACTGAGCGTGAGCCCTGGCGAGAGG |
| | | GCCAGCATCTCCTGCAGGGCTAGCCAAAGCCTGGTGCACAGCAACGGCA |
| | | ACACCCACCTGCACTGGTACCAGCAGAGACCCGGACAGGCTCCCAGGCT |
| | | GCTGATCTACAGCGTGAGCAACAGGTTCTCCGAGGTGCCTGCCAGGTTT |
| | | AGCGGCAGCGGAAGCGGCACCGACTTTACCCTGACCATCAGCAGCGTGG |
| | | AGTCCGAGGACTTCGCCGTGTATTACTGCAGCCAGACCAGCCACATCCC |
| | | TTACACCTTCGGCGGCGGCACCAAGCTGGAGATCAAAAGTGCTGCTGCC |
| | | TTTGTCCCGGTATTTCTCCCAGCCAAACCGACCACGACTCCCGCCCCGC |
| | | GCCCTCCGACACCCGCTCCCACCATCGCCTCTCAACCTCTTAGTCTTCG |
| | | CCCCGAGGCATGCCGACCCGCCGCCGGGGGTGCTGTTCATACGAGGGGC |
| | | TTGGACTTCGCTTGTGATATTTACATTTGGGCTCCGTTGGCGGGTACGT |
| | | GCGGCGTCCTTTTGTTGTCACTCGTTATTACTTTGTATTGTAATCACAG |
| | | GAATCGCAAACGGGGCAGAAAGAAACTCCTGTATATATTCAAACAACCA |
| | | TTTATGAGACCAGTACAAACTACTCAAGAGGAAGATGGCTGTAGCTGCC |
| | | GATTTCCAGAAGAAGAAGAAGGAGGATGTGAACTGCGAGTGAAGTTTTC |
| | | CCGAAGCGCAGACGCTCCGGCATATCAGCAAGGACAGAATCAGCTGTAT |
| | | AACGAACTGAATTTGGGACGCCGCGAGGAGTATGACGTGCTTGATAAAC |
| | | GCCGGGGGAGAGACCCGGAAATGGGGGGTAAACCCCGAAGAAAGAATCC |
| | | CCAAGAAGGACTCTACAATGAACTCCAGAAGGATAAGATGGCGGAGGCC |
| | | TACTCAGAAATAGGTATGAAGGGCGAACGACGACGGGGAAAAGGTCACG |
| | | ATGGCCTCTACCAAGGGTTGAGTACGGCAACCAAAGATACGTACGATGC |
| | | ACTGCATATGCAGGCCCTGCCTCCCAGATAATAATAAAATCGCTATCCA |
| | | TCGAAGATGGATGTGTGTTGGTTTTTTGTGTGTGGAGCAACAAATCTGA |
| | | CTTTGCATGTGCAAACGCCTTCAACAACAGCATTATTCCAGAAGACACC |
| | | TTCTTCCCCAGCCCAGGTAAGGGCAGCTTTGGTGCCTTCGCAGGCTGTT |
| | | TCCTTGCTTCAGGAATGGCCAGGTTCTGCCCAGAGCTCTGGTCAATGAT |
| | | GTCTAAAACTCCTCTGATTGGTGGTCTCGGCCTTATCCATTGCCACCAA |
| | | AACCCTCTTTTTACTAAGAAACAGTGAGCCTTGTTCTGGCAGTCCAGAG |
| | | AATGACACGGGAAAAAAGCAGATGAAGAGAAGGTGGCAGGAGAGGGCAC |
| | | GTGGCCCAGCCTCAGTCTCTCCAACTGAGTTCCTGCCTGCCTGCCTTTG |
| | | CTCAGACTGTTTGCCCCTTACTGCTCTTCTAGGCCTCATTCTAAGCCCC |
| | | TTCTCCAAGTTGCCTCTCCTTATTTCTCCCTGTCTGCCAAAAAATCTTT |
| | | CCCAGCTCACTAAGTCAGTCTCACGCAGTCACTCATTAACCCACCAATC |
| | | ACTGATTGTGCCGGCACATGAATGCACCAGGTGTTGAAGTGGAGGAATT |
| | | AAAAAGTCAGATGAGGGGTGTGCCCAGAGGAAGCACCATTCTAGTTGGG |
| | | GGAGCCCATCTGTCAGCTGGGAAAAGTCCAAATAACTTCAGATTGGAAT |
| | | GTGTTTTAACTCAGGGTTGAGAAAACAGCTACCTTCAGGACAAAAGTCA |
| | | GGGAAGGGCTCTCTGAAGAAATGCTACTTGAAGATACCAGCCCTACCAA |
| | | GGGCAGGGAGAGGACCCTATAGAGGCCTGGGACAGGAGCTCAATGAGAA |
| | | AGG |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

The term “about” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Where particular values are described in the application and claims, unless otherwise stated, the term “about” is implicit and in this context means within an acceptable error range for the particular value.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method for producing T cells expressing a chimeric antigen receptor (CAR-T cells), the method comprising: (i) culturing a first population of CAR-T cells in a medium comprising lenalidomide or a derivative thereof to produce a second population of CAR-T cells.

2. The method of claim 1, further comprising (ii) administering an effective amount of the second population of CAR-T cells produced in step (i) to a subject in need thereof.

3. The method of claim 2, wherein the CAR-T cells are allogenic to the subject.

4. The method of claim 1, wherein the CAR-T cells produced in the presence of lenalidomide exhibit one or more of the following improved features as compared with the same CAR-T cells cultured in the absence of lenalidomide:
(i) enhanced T cell proliferation and/or expansion capacity;
(ii) increased T cell number;
(ii) decreased senescence;
(iii) improved effector activity, which optionally is characterized by improved cytokine secretion upon antigen stimulation; and/or
(iv) improved cytotoxicity.

5. The method of claim 1, wherein the chimeric antigen receptor (CAR) expressed in the CAR-T cells comprises an extracellular antigen binding domain, which optionally is a single chain variable fragment (scFv), a co-stimulatory signaling domain of 4-1BB or CD28, and a cytoplasmic signaling domain of CD3ζ.

6. The method of claim 5, wherein the extracellular antigen binding domain is specific to a tumor antigen.

7. The method of claim 6,
wherein the extracellular antigen binding domain is a single chain variable fragment (scFv) that binds CD19, which comprises the amino acid sequence of SEQ ID NO: 104;
wherein the extracellular antigen binding domain is a single chain variable fragment (scFv) that binds BCMA, which comprises the amino acid sequence of SEQ ID NO: 133; or
wherein the extracellular antigen binding domain is a single chain variable fragment (scFv) that binds CD70, which comprises the amino acid sequence of SEQ ID NO: 127.

8. The method of claim 6,
wherein the CAR that binds CD19 comprises the amino acid sequence of SEQ ID NO: 102;
wherein the CAR that binds BCMA comprises the amino acid sequence of SEQ ID NO: 131; or
wherein the CAR that binds CD70 comprises the amino acid sequence of SEQ ID NO: 123.

9. The method of claim 1, wherein the nucleic acid encoding the CAR is inserted in a genomic site in the CAR-T cells.

10. The method of claim 1, wherein the CAR-T cells have a disrupted TRAC gene, a disrupted β2M gene, or both.

11. The method of claim 9, wherein the CAR-T cells have a disrupted TRAC gene, which comprises a deletion of a fragment having the nucleotide sequence of SEQ ID NO: 29.

12. The method of claim 11, wherein the nucleic acid encoding the CAR is inserted in the disrupted TRAC gene.

13. The method of claim 12, wherein the nucleic acid encoding the CAR substitutes for the fragment of SEQ ID NO: 29.

14. The method of claim 13, wherein the disrupted TRAC gene comprises the nucleotide sequence of SEQ ID NO: 153, SEQ ID NO: 154, or SEQ ID NO: 155

15. The method of claim 10, wherein the CAR-T cells comprise the disrupted TRAC gene and the disrupted β2M gene.

16. The method of claim 10, wherein the CAR-T cells further comprise a disrupted CD70 gene, a disrupted Regnase-1 (Reg1) gene, a disrupted TGFBRII gene, a disrupted TET2 gene, or a combination thereof.

17. The method of claim 16, wherein the CAR comprises an extracellular antigen binding domain that binds CD70, and wherein the CAR-T cells comprise a disrupted CD70 gene.

18. The method of claim 16, wherein the disrupted TRAC gene, the disrupted β2M gene, the disrupted CD70 gene, the disrupted Reg1 gene, and/or the disrupted TGFBRII gene are produced by a CRISPR/Cas gene editing system.

19. The method of claim 18, wherein the disrupted TRAC gene is targeted by an sgRNA comprising the nucleotide sequence of SEQ ID NO: 5, the disrupted B2M gene is targeted by an sgRNA comprising the nucleotide sequence of SEQ ID NO: 9, the disrupted CD70 gene is targeted by an sgRNA comprising the nucleotide sequence of SEQ ID NO: 13, the disrupted Reg1 gene is targeted by an sgRNA comprising the nucleotide sequence of SEQ ID NO: 17, the disrupted TET2 gene is targeted by an sgRNA comprising the nucleotide sequence of SEQ ID NO: 24; and/or the disrupted TGFBRII gene is targeted by an sgRNA comprising the nucleotide sequence of SEQ ID NO:21.

20. The method of claim 2, further comprising administering to the subject an effective amount of lenalidomide or a derivative thereof.

21. The method of claim 2, wherein the subject is a human cancer patient, who optionally has a cancer involving CD19⁺, BCMA⁺, or CD70⁺ cancer cells.

22. A method for eliminating undesired cells in a subject, the method comprising (a) administering an effective amount of allogenic T cells expressing a chimeric antigen receptor (CAR-T cells) to a subject in need thereof, and (b) administering to the subject an effective amount of lenalidomide or a derivative thereof.

23. The method of claim 22, wherein the CAR comprises an extracellular antigen binding domain, which optionally is a single chain variable fragment (scFv), a co-stimulatory signaling domain of 4-1BB or CD28, and a cytoplasmic signaling domain of CD3ζ.

24. The method of claim 22, wherein the extracellular antigen binding domain is tumor antigen.

25. The method of claim 24,
wherein the extracellular antigen binding domain is a single chain variable fragment (scFv) that binds CD19, which comprises the amino acid sequence of SEQ ID NO: 104;
wherein the extracellular antigen binding domain is a single chain variable fragment (scFv) that binds BCMA, which comprises the amino acid sequence of SEQ ID NO: 133; or
wherein the extracellular antigen binding domain is a single chain variable fragment (scFv) that binds CD70, which comprises the amino acid sequence of SEQ ID NO: 127.

26. The method of claim 25,
wherein the CAR that binds CD19 comprises the amino acid sequence of SEQ ID NO: 102;
wherein the CAR that binds BCMA comprises the amino acid sequence of SEQ ID NO: 131; or
wherein the CAR that binds CD70 comprises the amino acid sequence of SEQ ID NO: 123.

27. The method of claim 22, wherein the nucleic acid encoding the CAR is inserted in a genomic site in the CAR-T cells.

28. The method of claim 22, wherein the CAR-T cells have a disrupted TRAC gene, a disrupted β2M gene, or both.

29. The method of claim 28, wherein the CAR-T cells have a disrupted TRAC gene, which comprises a deletion of a fragment having the nucleotide sequence of SEQ ID NO: 29.

30. The method of claim 29, wherein the nucleic acid encoding the CAR is inserted in the disrupted TRAC gene.

31. The method of claim 30, wherein the nucleic acid encoding the CAR substitutes for the fragment of SEQ ID NO: 29.

32. The method of claim 31, wherein the disrupted TRAC gene comprises the nucleotide sequence of SEQ ID NO: 153, SEQ ID NO: 154, or SEQ ID NO: 155.

33. The method of claim 28, wherein the CAR-T cells comprise the disrupted TRAC gene and the disrupted β2M gene.

34. The method of claim 33, wherein the CAR-T cells further comprise a disrupted CD70 gene, a disrupted Regnase-1 (Reg1) gene, a disrupted TGFBRII gene, a disrupted TET2 gene, or a combination thereof.

35. The method of claim 22, wherein the CAR comprises an extracellular antigen binding domain that binds CD70 and wherein the CAR-T cells comprise a disrupted CD70 gene.

36. The method of claim 34, wherein the disrupted TRAC gene, the disrupted β2M gene, the disrupted CD70 gene, the disrupted Reg1 gene, and/or the disrupted TGFBRII gene are produced by a CRISPR/Cas gene editing system.

37. The method of claim 36, wherein the disrupted TRAC gene is targeted by an sgRNA comprising the nucleotide sequence of SEQ ID NO: 5, the disrupted B2M gene is targeted by an sgRNA comprising the nucleotide sequence of SEQ ID NO: 9, the disrupted CD70 gene is targeted by an sgRNA comprising the nucleotide sequence of SEQ ID NO: 13, the disrupted Reg1 gene is targeted by an sgRNA comprising the nucleotide sequence of SEQ ID NO: 17, the disrupted TET gene is targeted by an sgRNA comprising the nucleotide sequence of SEQ ID NO: 25; and/or the disrupted TGFBRII gene is targeted by an sgRNA comprising the nucleotide sequence of SEQ ID NO:21.

38. The method of claim 22, wherein the subject is a human cancer patient, who optionally has a cancer comprising CD19⁺, BCMA⁺, or CD70⁺ cells.

39. A method for improving treatment efficacy of T cells expressing a chimeric antigen receptor (CAR-T cells), the method comprising: administering an effective amount of CAR-T cells to a subject in need thereof, wherein the CAR-T cells have been cultured in vitro in the presence of lenalidomide or a derivative thereof.

40. The method of claim 39, wherein the CAR-T cells are allogeneic to the subject.

41. The method of claim 39, wherein the CAR comprises an extracellular antigen binding domain, which optionally is a single chain variable fragment (scFv), a co-stimulatory signaling domain of 4-1BB or CD28, and a cytoplasmic signaling domain of CD3ζ.

42. The method of claim 39, wherein the extracellular antigen binding domain is tumor antigen.

43. The method of claim 42,
wherein the extracellular antigen binding domain is a single chain variable fragment (scFv) that binds CD19, which comprises the amino acid sequence of SEQ ID NO: 104;
wherein the extracellular antigen binding domain is a single chain variable fragment (scFv) that binds BCMA, which comprises the amino acid sequence of SEQ ID NO: 133; or
wherein the extracellular antigen binding domain is a single chain variable fragment (scFv) that binds CD70, which comprises the amino acid sequence of SEQ ID NO: 127.

44. The method of claim 43,
wherein the CAR that binds CD19 comprises the amino acid sequence of SEQ ID NO: 102;
wherein the CAR that binds BCMA comprises the amino acid sequence of SEQ ID NO: 131; or
wherein the CAR that binds CD70 comprises the amino acid sequence of SEQ ID NO: 123.

45. The method of claim 39, wherein the nucleic acid encoding the CAR is inserted in a genomic site in the CAR-T cells.

46. The method of claim 39, wherein the CAR-T cells have a disrupted TRAC gene, a disrupted β2M gene, or both.

47. The method of claim 46, wherein the CAR-T cells have a disrupted TRAC gene, which comprises a deletion of a fragment having the nucleotide sequence of SEQ ID NO: 29.

48. The method of claim 47, wherein the nucleic acid encoding the CAR is inserted in the disrupted TRAC gene.

49. The method of claim 48, wherein the nucleic acid encoding the CAR substitutes for the fragment of SEQ ID NO: 29.

50. The method of claim 49, wherein the disrupted TRAC gene comprises the nucleotide sequence of SEQ ID NO: 153, SEQ ID NO: 154, or SEQ ID NO: 155.

51. The method of claim 46, wherein the CAR-T cells comprise the disrupted TRAC gene and the disrupted β2M gene.

52. The method of claim 46, wherein the CAR-T cells further comprise a disrupted CD70 gene, a disrupted Regnase-1 (Reg1) gene, a disrupted TGFBRII gene, a disrupted TET2 gene, or a combination thereof.

53. The method of claim 39, wherein the CAR comprises an extracellular antigen binding domain that binds CD70 and wherein the CAR-T cells comprise a disrupted CD70 gene.

54. The method of claim 52, wherein the disrupted TRAC gene, the disrupted β2M gene, the disrupted CD70 gene, the disrupted Reg1 gene, and/or the disrupted TGFBRII gene are produced by a CRISPR/Cas gene editing system.

55. The method of claim 54, wherein the disrupted TRAC gene is targeted by an sgRNA comprising the nucleotide sequence of SEQ ID NO: 5, the disrupted B2M gene is targeted by an sgRNA comprising the nucleotide sequence of SEQ ID NO: 9, the disrupted CD70 gene is targeted by an sgRNA comprising the nucleotide sequence of SEQ ID NO: 13, the disrupted Reg1 gene is targeted by an sgRNA comprising the nucleotide sequence of SEQ ID NO: 17, the disrupted TET gene is targeted by an sgRNA comprising the nucleotide sequence of SEQ ID NO: 25; and/or the disrupted TGFBRII gene is targeted by an sgRNA comprising the nucleotide sequence of SEQ ID NO:21.

56. The method of claim 39, wherein the subject is a human cancer patient, who optionally has a cancer comprising CD19⁺, BCMA⁺, or CD70⁺ cells.

57. A kit for use in cancer therapy, the kit comprising:
(i) a population of T cells expressing a chimeric antigen receptor (CAR-T cells); and
(ii) lenalidomide or a derivative thereof.

58. The kit of claim 57, wherein the CAR-T cells express a chimeric antigen receptor (CAR), which comprises an extracellular antigen binding domain, a co-stimulatory signaling domain of 4-1BB or CD28, and a cytoplasmic signaling domain of CD3ζ.
